(19) United States
(12) Patent Application Publication
(10) Pub. No.: US 2006/0269946 A1
(43) Pub. Date: Nov. 30, 2006

(54) Methods and compositions for assessment of pulmonary function and disorders

(76) Inventor: Robert Peter Young, Parnell (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 11/432,736

(22) Filed: May 10, 2006

(30) Foreign Application Priority Data
May 10, 2005 (NZ) 539934
Aug. 19, 2005 (NZ) 541935
Dec. 14, 2005 (JP) 2005-360523

(51) Int. Cl. C12Q 1/68

(52) U.S. Cl. 435/6

(57) ABSTRACT

The present invention provides methods for the assessment of risk of developing chronic obstructive pulmonary disease (COPD), emphysema or both COPD and emphysema in smokers and non-smokers using analysis of genetic polymorphisms. The present invention also relates to the use of genetic polymorphisms in assessing a subject's risk of developing COPD, emphysema or both COPD and emphysema. Furthermore, methods and compositions for the treatment or prevention of these issues are also provided.

### RELATED APPLICATIONS

This application claims priority to: New Zealand Application No. 539934, filed May 10, 2005; New Zealand Application No. 541935, filed Aug. 19, 2005; and Japanese Application No. 2005-360523, filed Dec. 14, 2005, all of which are incorporated by reference in their entireties.

### FIELD OF THE INVENTION

The present invention is concerned with methods for assessment of pulmonary function and/or disorders, and in particular for assessing risk of developing chronic obstructive pulmonary disease (COPD) and emphysema in smokers and non-smokers using analysis of genetic polymorphisms and altered gene expression. The present invention is also concerned with the use of genetic polymorphisms in the assessment of a subject's risk of developing COPD and emphysema.

### BACKGROUND OF THE INVENTION

Chronic obstructive pulmonary disease (COPD) is the 4^{th} leading cause of death in developed countries and a major cause for hospital readmission world-wide. It is characterised by insidious inflammation and progressive lung destruction. It becomes clinically evident after exertional breathlessness is noted by affected smokers when 50% or more of lung function has already been irreversibly lost. This loss of lung function is detected clinically by reduced expiratory flow rates (specifically forced expiratory volume in one second or FEV1). Over 95% of COPD is attributed to cigarette smoking yet only 20% or so of smokers develop COPD (susceptible smoker). Studies surprisingly show that smoking dose accounts for only about 16% of the impaired lung function. A number of family studies comparing concordance in siblings (twins and non-twin) consistently show a strong familial tendency and the search for COPD disease-susceptibility (or disease modifying) genes is underway.

Despite advances in the treatment of airways disease, current therapies do not significantly alter the natural history of COPD with progressive loss of lung function causing respiratory failure and death. Although cessation of smoking has been shown to reduce this decline in lung function if this is not achieved within the first 20 years or so of smoking for susceptible smokers, the loss is considerable and symptoms of worsening breathlessness cannot be averted. Smoking cessation studies indicate that techniques to help smokers quit have limited success. Analogous to the discovery of serum cholesterol and its link to coronary artery disease, there is a need to better understand the factors that contribute to COPD so that tests that identify at risk smokers can be developed and that new treatments can be discovered to reduce the adverse effects of smoking.

A number of epidemiology studies have consistently shown that at exposure doses of 20 or more pack years, the distribution in lung function tends toward trimodality with a proportion of smokers maintaining normal lung function (resistant smokers) even after 60+ pack years, a proportion showing modest reductions in lung function who may never develop symptoms and a proportion who show an accelerated loss in lung function who invariably develop COPD. This suggests that amongst smokers 3 populations exist, those resistant to developing COPD, those at modest risk and those at higher risk (termed susceptible smokers).

COPD is a heterogeneous disease encompassing, to varying degrees, emphysema and chronic bronchitis which develop as part of a remodelling process following the inflammatory insult from chronic tobacco smoke exposure and other air pollutants. It is likely that many genes are involved in the development of COPD.

To date, a number of biomarkers useful in the diagnosis and assessment of propensity towards developing various pulmonary disorders have been identified. These include, for example, single nucleotide polymorphisms including the following: A-82G in the promoter of the gene encoding human macrophage elastase (MMP12); T→C within codon 10 of the gene encoding transforming growth factor beta (TGFβ); C+760G of the gene encoding superoxide dismutase 3 (SOD3); T-1296C within the promoter of the gene encoding tissue inhibitor of metalloproteinase 3 (TIMP3); and polymorphisms in linkage disequilibrium (LD) with these polymorphisms, as disclosed in PCT International Application PCT/NZ02/00106 (published as WO 02/099134 and incorporated by reference herein in its entirety).

### SUMMARY OF THE INVENTION

It can be desirable and advantageous to have additional biomarkers that can be used to assess a subject's risk of developing pulmonary disorders such as chronic obstructive pulmonary disease (COPD) and emphysema, or a risk of developing COPD/emphysema-related impaired lung function, particularly if the subject is a smoker. In some embodiments, it is to such biomarkers and their use in methods to assess risk of developing such disorders that the present invention is directed.

In some aspects, the present invention is primarily based on the finding that certain polymorphisms are found more often in subjects with COPD, emphysema, or both COPD and emphysema than in control subjects. Analysis of these polymorphisms reveals an association between genotypes and the subject's risk of developing COPD, emphysema, or both COPD and emphysema.

Thus, according to one aspect there is provided a method of determining a subject's risk of developing one or more obstructive lung diseases comprising analysing a sample from said subject for the presence or absence of one or more polymorphisms selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin; and
−1607 1G/2G in the promoter of the gene encoding Matrix Metalloproteinase 1 (MMP1), with reference to the 1G allele only;
wherein the presence or absence of one or more of said polymorphisms is indicative of the subject's risk of developing one or more obstructive lung diseases selected from the group consisting of chronic obstructive pulmonary disease (COPD), emphysema, or both COPD, emphysema, or both COPD and emphysema.

The one or more polymorphisms can be detected directly or by detection of one or more polymorphisms which are in linkage disequilibrium with said one or more polymorphisms.

Linkage disequilibrium (LD) is a phenomenon in genetics whereby two or more mutations or polymorphisms are in such close genetic proximity that they are co-inherited. This means that in genotyping, detection of one polymorphism as present implies the presence of the other. (Reich D E et al; Linkage disequilibrium in the human genome, Nature 2001, 411:199-204 (2001), herein incorporated by reference in its entirety).

The method can additionally comprise analysing a sample from said subject for the presence of one or more further polymorphisms selected from the group consisting of:
16Arg/Gly in the gene encoding β2 Adrenergic Receptor (ADBR);
130 Arg/Gln (G/A) in the gene encoding Interleukin13 (IL13);
298 Asp/Glu (T/G) in the gene encoding Nitric oxide Synthase 3 (NOS3);
Ile 105 Val (A/G) in the gene encoding Glutathione S Transferase P (GST-P);
Glu 416 Asp (T/G) in the gene encoding Vitamin D binding protein (VDBP);
Lys 420 Thr (A/C) in the gene encoding VDBP;
−1055 C/T in the promoter of the gene encoding IL13;
−308 G/A in the promoter of the gene encoding TNFα;
−511 A/G in the promoter of the gene encoding Interleukin 1B (IL1B);
Tyr 113 His T/C in the gene encoding Microsomal epoxide hydrolase (MEH);
His139 Arg G/A in the gene encoding MEH;
Gln 27 Glu C/G in the gene encoding ADBR;
−1607 1G/2G in the promoter of the gene encoding Matrix Metalloproteinase 1 (MMP1) with reference to the 2G allel only;
−1562 C/T in the promoter of the gene encoding Metalloproteinase 9 (MMP9);
M1 (GSTM1) null in the gene encoding Glutathione S Transferase 1 (GST-1);
1237 G/A in the 3′ region of the gene encoding α1-antitrypsin;
−82 A/G in the promoter of the gene encoding MMP12;
T→C within codon 10 of the gene encoding TGFβ;
760 C/G in the gene encoding SOD3;
−1296 T/C within the promoter of the gene encoding TIMP3; and
the S mutation in the gene encoding α1-antitrypsin.

Again, detection of the one or more further polymorphisms can be carried out directly or by detection of polymorphisms in linkage disequilibrium with the one or more further polymorphisms.

The presence of one or more polymorphisms selected from the group consisting of:
the −765 CC or CG genotype in the promoter of the gene encoding COX2;
the 130 Arg/Gln AA genotype in the gene encoding IL13;
the 298 Asp/Glu TT genotype in the gene encoding NOS3;
the Lys 420 Thr AA or AC genotype in the gene encoding VDBP;
the Glu 416 Asp TT or TG genotype in the gene encoding VDBP;
the Ile 105 Val AA genotype in the gene encoding GSTP-1;
the MS genotype in the gene encoding α1-antitrypsin;
the +489 GG geneotype in the gene encoding TNFα;
the −308 GG geneotype in the gene encoding TNFα;
the C89Y AA or AG geneotype in the gene encodoing SMAD3;
the 161 GG genotype in the gene encodoing MBL2;
the −1903 AA genotype in the gene encoding CMA1;
the Arg 197 Gln AA genotype in the gene encoding NAT2;
the His 139 Arg GG genotype in the gene encoding MEH;
the −366 AA or AG genotype in the gene encoding ALOX5;
the HOM T2437C TT genotype in the gene encoding HSP 70;
the exon 1 +49 CT or TT genotype in the gene encoding Elafin;
the Gln 27 Glu GG genotype in the gene encoding ADBR; or
the −1607 1G1G or 1G2G genotype in the promoter of the gene encoding MMP1; can be indicative of a reduced risk of developing COPD, emphysema, or both COPD and emphysema.

The presence of one or more polymorphisms selected from the group consisting of:
the 105 AA genotype in the gene encoding IL18;
the −133 CC genotype in the promoter of the gene encoding IL18;
the −675 5G5G genotype in the promoter of the gene encoding PAI-1;
the −1055 TT genotype in the promoter of the gene encoding IL13;
the 874 TT genotype in the gene encoding IFN-γ;
the +489 AA or AG genotype in the gene encoding TNFα;
the −308 AA or AG genotype in the gene encoding TNFα;
the C89Y GG genotype in the gene encoding SMAD3;
the E469K GG genotype in the gene encoding ICAM1;
the Gly 881 Arg GC or CC genotype in the gene encoding NOD2;
the −511 GG genotype in the gene encoding IL1B;
the Tyr 113 His TT genotype in the gene encoding MEH;
the −366 GG genotype in the gene encoding ALOX5;
the HOM T2437C CC or CT genotype in the gene encoding HSP 70;
the +13924 AA genotype in the gene encoding CLCA1; and
the −159 CC genotype in the gene encoding CD-14;
   can be indicative of an increased risk of developing COPD, emphysema, or both COPD and emphysema.

The methods of the invention are particularly useful in smokers (both current and former).

It will be appreciated that the methods of the invention identify two categories of polymorphisms—namely those associated with a reduced risk of developing COPD, emphysema, or both COPD and emphysema (which can be termed “protective polymorphisms”) and those associated with an increased risk of developing COPD, emphysema, or both COPD and emphysema (which can be termed “susceptibility polymorphisms”).

Therefore, the present invention further provides a method of assessing a subject's risk of developing chronic obstructive pulmonary disease (COPD), emphysema, or both COPD and emphysema, said method comprising:
determining the presence or absence of at least one protective polymorphism associated with a reduced risk of developing COPD, emphysema, or both COPD and emphysema; and
in the absence of at least one protective polymorphism, determining the presence or absence of at least one susceptibility polymorphism associated with an increased risk of developing COPD, emphysema, or both COPD and emphysema;
wherein the presence of one or more of said protective polymorphisms is indicative of a reduced risk of developing COPD, emphysema, or both COPD and emphysema, and the absence of at least one protective polymorphism in combination with the presence of at least one susceptibility polymorphism is indicative of an increased risk of developing COPD, emphysema, or both COPD and emphysema.

Preferably, said at least one protective polymorphism is selected from the group consisting of:
−765 C in the promoter of the gene encoding COX2;
130 Arg/Gln A in the gene encoding IL13;
298 Asp/Glu T in the gene encoding NOS3;
Lys 420 Thr A in the gene encoding VDBP;
Glu 416 Asp T in the gene encoding VDBP;
Ile 105 Val A in the gene encoding GSTP-1;
the S mutation in the gene encoding α1-antitrypsin;
+489 G in the gene encoding TNFα;
−308 G in the gene encoding TNFα;
C89Y A in the gene encoding SMAD3;
161 G in the gene encoding MBL2;
−1903 A in the gene encoding CMA1;
Arg 197 Gln A in the gene encoding NAT2;
His 139 Arg G in the gene encoding MEH;
−366 A in the gene encoding ALOX5;
HOM 2437 T in the gene encoding HSP 70;
exon 1 +49 T in the gene encodoing Elafin;
Gln 27 Glu G in the gene encoding ADBR; and
−1607 1G in the promoter of the gene encoding MMP1.

In another embodiment, said at least one protective polymorphism is a genotype selected from the group consisting of:
the −765 CC or CG genotype in the promoter of the gene encoding COX2;
the 130 Arg/Gln AA genotype in the gene encoding IL13;
the 298 Asp/Glu TT genotype in the gene encoding NOS3;
the Lys 420 Thr AA or AC genotype in the gene encoding VDBP;
the Glu 416 Asp TT or TG genotype in the gene encoding VDBP;
the Ile 105 Val AA genotype in the gene encoding GSTP-1;
the MS genotype in the gene encoding α1-antitrypsin;
the +489 GG geneotype in the gene encoding TNFα;
the −308 GG geneotype in the gene encoding TNFα;
the C89Y AA or AG geneotype in the gene encodoing SMAD3;
the 161 GG genotype in the gene encodoing MBL2;
the −1903 AA genotype in the gene encoding CMA1;
the Arg 197 Gln AA genotype in the gene encoding NAT2;
the His 139 Arg GG genotype in the gene encoding MEH;
the −366 AA or AG genotype in the gene encoding ALOX5;
the HOM T2437C TT genotype in the gene encoding HSP 70;
the exon 1 +49 CT or TT genotype in the gene encoding Elafin;
the Gln 27 Glu GG genotype in the gene encoding ADBR; and
the −1607 1G1G or 1G2G genotype in the promoter of the gene encoding MMP1.

Optionally, said method includes the additional step of determining the presence or absence of at least one further protective polymorphism selected from the group consisting of:
the +760GG or +760CG genotype within the gene encoding SOD3;
the −1296TT genotype within the promoter of the gene encoding TIMP3; or
the CC genotype (homozygous P allele) within codon 10 of the gene encoding TGFβ.

The at least one susceptibility polymorphism can be a genotype selected from the group consisting of:
the 105 AA genotype in the gene encoding IL18;
the −133 CC genotype in the promoter of the gene encoding IL18;
the −675 5G5G genotype in the promoter of the gene encoding PAI-1;
the −1055 TT genotype in the promoter of the gene encoding IL13;
the 874 TT genotype in the gene encoding IFN-γ;
the +489 AA or AG genotype in the gene encoding TNFα;
the −308 AA or AG genotype in the gene encoding TNFα;
the C89Y GG genotype in the gene encoding SMAD3;
the E469K GG genotype in the gene encoding ICAM1;
the Gly 881 Arg GC or CC genotype in the gene encoding NOD2;
the −511 GG genotype in the gene encoding IL1B;
the Tyr 113 His TT genotype in the gene encoding MEH;
the −366 GG genotype in the gene encoding ALOX5;
the HOM T2437C CC or CT genotype in the gene encoding HSP 70;
the +13924 AA genotype in the gene encoding CLCA1; and
the −159 CC genotype in the gene encoding CD-14.

Optionally, said method includes the step of determining the presence or absence of at least one further susceptibility polymorphism selected from the group consisting of:
the −82AA genotype within the promoter of the gene encoding MMP12;
the −1562CT or −1562TT genotype within the promoter of the gene encoding MMP9;
the 1237AG or 1237AA genotype (Tt or tt allele genotypes) within the 3′ region of the gene encoding α1-antitrypsin; and
the 2G2G genotype within the promoter of the gene encoding MMP1.

In a preferred form of the invention the presence of two or more protective polymorphisms is indicative of a reduced risk of developing COPD, emphysema, or both COPD and emphysema.

In a further preferred form of the invention the presence of two or more susceptibility polymorphisms is indicative of an increased risk of developing COPD, emphysema, or both COPD and emphysema.

In still a further preferred form of the invention the presence of two or more protective polymorphims irrespective of the presence of one or more susceptibility polymorphisms is indicative of reduced risk of developing COPD, emphysema, or both COPD and emphysema.

In another aspect, the invention provides a method of determining a subject's risk of developing COPD, emphysema, or both COPD and emphysema, said method comprising obtaining the result of one or more genetic tests of a sample from said subject, and analysing the result for the presence or absence of one or more polymorphisms selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881 Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding Matrix Metalloproteinase 1 (MMP1), with reference to the 1G allele only;
and one or more polymorphisms which are in linkage disequilibrium with any one or more of these polymorphisms;
wherein a result indicating the presence or absence of one or more of said polymorphisms is indicative of the subject's risk of developing COPD, emphysema, or both COPD and emphysema.

In a further aspect the invention provides a method of determining a subject's risk of developing chronic obstructive pulmonary disease (COPD), emphysema, or both COPD and emphysema, said method comprising determining the presence or absence of the −765 C allele in the promoter of the gene encoding COX2 and/or the S allele in the gene encoding 1-antitrypsin, wherein the presence of any one or more of said alleles is indicative of a reduced risk of developing COPD, emphysema, or both COPD and emphysema.

In a further aspect the invention provides a method of determining a subject's risk of developing chronic obstructive pulmonary disease (COPD), emphysema, or both COPD and emphysema, said method comprising determining the presence or absence of the −765 CC or CG genotype in the promoter of the gene encoding COX2 and/or the MS genotype in the gene encoding 1-antitrypsin, wherein the presence of any one or more of said genotypes is indicative of a reduced risk of developing COPD, emphysema, or both COPD and emphysema.

In one particularly preferred form of the invention there is provided a method of determining a subject's risk of developing chronic obstructive pulmonary disease (COPD), emphysema, or both COPD and emphysema, comprising the analysis of one or more polymorphisms selected from the group consisting of:
−765 C/G in the promoter of the gene encoding COX2;
105 C/A in the gene encoding IL18;
−133 G/C in the promoter of the gene encoding IL18;
−675 4G/5G in the promoter of the gene encoding PAI-1;
874 A/T in the gene encoding IFN-γ;
+489 G/A in the gene encoding TNFα;
C89Y A/G in the gene encoding SMAD3;
E 469 K A/G in the gene encoding ICAM1;
Gly 881Arg G/C in the gene encoding NOD2;
161 G/A in the gene encoding MBL2;
−1903 G/A in the gene encoding CMA1;
Arg 197 Gln G/A in the gene encoding NAT2;
−366 G/A in the gene encoding ALOX5;
HOM T2437C in the gene encoding HSP 70;
+13924 T/A in the gene encoding CLCA1;
−159 C/T in the gene encoding CD-14;
exon 1 +49 C/T in the gene encoding Elafin; and
−1607 1G/2G in the promoter of the gene encoding MMP1 (with reference to the 1G allele only)

in combination with one or more polymorphisms selected from the group consisting of:
16Arg/Gly in the gene encoding ADBR;
130 Arg/Gln (G/A) in the gene encoding IL13;
298 Asp/Glu (T/G) in the gene encoding NOS3;
Ile 105 Val (A/G) in the gene encoding GSTP;
Glu 416 Asp (T/G) in the gene encoding VDBP;
Lys 420 Thr (A/C) in the gene encoding VDBP;
−1055 C/T in the promoter of the gene encoding IL13;
the S mutation in the gene encoding α1-antitrypsin;
−308 G/A in the promoter of the gene encoding TNFα;
−511 A/G in the promoter of the gene encoding IL1B;
Tyr 113 His T/C in the gene encoding MEH;
His 139 Arg G/A in the gene encoding MEH; and
Gln 27 Glu C/G in the gene encoding ADBR.

In a further aspect there is provided a method of determining a subject's risk of developing chronic obstructive pulmonary disease (COPD), emphysema, or both COPD and emphysema, comprising the analysis of two or more polymorphisms selected from the group consisting of:
−765 C/G in the promoter of the gene encoding COX2;
105 C/A in the gene encoding IL18;
−133 G/C in the promoter of the gene encoding IL18;
−b 675 4G/5G in the promoter of the gene encoding PAI-1;
874 A/T in the gene encoding IFN-γ;
16Arg/Gly in the gene encoding ADBR;
130 Arg/Gln (G/A) in the gene encoding IL13;
298 Asp/Glu (T/G) in the gene encoding NOS3;
Ile 105 Val (A/G) in the gene encoding glutathione S transferase P (GST-P);
Glu 416 Asp (T/G) in the gene encoding VDBP;
Lys 420 Thr (A/C) in the gene encoding VDBP;
−1055 C/T in the promoter of the gene encoding IL13;
the S mutation in the gene encoding α1-antitrypsin;
+489 G/A in the gene encoding TNFα;
C89Y A/G in the gene encoding SMAD3;
E 469 K A/G in the gene encoding ICAM1;
Gly 881Arg G/C in the gene encoding NOD2;
161 G/A in the gene encoding MBL2;
−1903 G/A in the gene encoding CMA1;
Arg 197 Gln G/A in the gene encoding NAT2;
−366 G/A in the gene encoding ALOX5;
HOM T2437C in the gene encoding HSP 70;
+13924 T/A in the gene encoding CLCA1;
−159 C/T in the gene encoding CD-14;
exon 1 +49 C/T in the gene encoding Elafin;
−308 G/A in the promoter of the gene encoding TNFα;
−511 A/G in the promoter of the gene encoding IL1B;
Tyr 113 His T/C in the gene encoding MEH;
Arg 139 G/A in the gene encoding MEH;
Gln 27 Glu C/G in the gene encoding ADBR; and
−1607 1G/2G in the promoter of the gene encoding MMP1 (with reference to the 1G allele only).

In various embodiments, any one or more of the above methods includes the step of analysing the amino acid present at a position mapping to codon 298 of the gene encoding NOS3.

The presence of glutamate at said position is indicative of an increased risk of developing COPD, emphysema, or both COPD and emphysema.

The presence of asparagine at said position is indicative of reduced risk of developing COPD, emphysema, or both COPD and emphysema.

In various embodiments, any one or more of the above methods includes the step of analysing the amino acid present at a position mapping to codon 420 of the gene encoding vitamin D binding protein.

The presence of threonine at said position is indicative of an increased risk of developing COPD, emphysema, or both COPD and emphysema.

The presence of lysine at said position is indicative of reduced risk of developing COPD, emphysema, or both COPD and emphysema.

In various embodiments, any one or more of the above methods includes the step of analysing the amino acid present at a position mapping to codon 89 of the gene encoding SMAD3.

In various embodiments, any one or more of the above methods includes the step of analysing the amino acid present at a position mapping to codon 469 of the gene encoding ICAM1.

In various embodiments, any one or more of the above methods includes the step of analysing the amino acid present at a position mapping to codon 881 of the gene encoding NOD2.

In various embodiments, any one or more of the above methods includes the step of analysing the amino acid present at a position mapping to codon 197 of the gene encoding NAT2.

In various embodiments, any one or more of the above methods includes the step of analysing the amino acid present at a position mapping to codon 113 of the gene encoding MEH.

In various embodiments, any one or more of the above methods includes the step of analysing the amino acid present at a position mapping to codon 139 of the gene encoding MEH.

In various embodiments, any one or more of the above methods includes the step of analysing the amino acid present at a position mapping to codon 27 of the gene encoding ADBR.

In a preferred form of the invention the methods as described herein are performed in conjunction with an analysis of one or more risk factors, including one or more epidemiological risk factors, associated with a risk of developing chronic obstructive pulmonary disease (COPD) and/or emphysema. Such epidemiological risk factors include but are not limited to smoking or exposure to tobacco smoke, age, sex, and familial history of COPD, emphysema, or both COPD and emphysema.

In a further aspect, the invention provides for the use of at least one polymorphism in the assessment of a subject's risk of developing COPD, emphysema, or both COPD and emphysema, wherein said at least one polymorphism is selected from the group consisting of:
−765 C/G in the promoter of the gene encoding Cyclooxygenase 2 (COX2);
105 C/A in the gene encoding Interleukin18 (IL8);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding Matrix Metalloproteinase 1 (MMP1), with reference to the 1G allele only; and
one or more polymorphisms in linkage disequilibrium with any one of said polymorphisms.

Optionally, said use can be in conjunction with the use of at least one further polymorphism selected from the group consisting of:
16Arg/Gly in the gene encoding ADBR;
130 Arg/Gln (G/A) in the gene encoding IL13;
298 Asp/Glu (T/G) in the gene encoding NOS3;
Ile 105 Val (A/G) in the gene encoding GSTP;
Glu 416 Asp (T/G) in the gene encoding VDBP;
Lys 420 Thr (A/C) in the gene encoding VDBP;
−1055 C/T in the promoter of the gene encoding IL13;
the S mutation in the gene encoding α1-antitrypsin;
−308 G/A in the promoter of the gene encoding TNFα;
−511 A/G in the promoter of the gene encoding IL1B;
Tyr 113 His T/C in the gene encoding MEH;
His 139 Arg G/A in the gene encoding MEH;
Gln 27 Glu C/G in the gene encoding ADBR;
−1607 1G/2G in the promoter of the gene encoding MMP1;
−1562 C/T in the promoter of the gene encoding MMP9;
M1 (GSTM1) null in the gene encoding GST-1;
1237 G/A in the 3′ region of the gene encoding α1-antitrypsin;
−82 A/G in the promoter of the gene encoding MMP12;
T→C within codon 10 of the gene encoding TGFβ;
760 C/G in the gene encoding SOD3;
−1296 T/C within the promoter of the gene encoding TIMP3; and
the S mutation in the gene encoding α1-antitrypsin.

In another aspect the invention provides a set of nucleotide probes and/or primers for use in the preferred methods of the invention herein described. Preferably, the nucleotide probes and/or primers are those which span, or are able to be used to span, the polymorphic regions of the genes.

In yet a further aspect, the invention provides a nucleic acid microarray for use in the methods of the invention, which microarray includes a substrate presenting nucleic acid sequences capable of hybridizing to nucleic acid sequences which encode one or more of the susceptibility or protective polymorphisms described herein or sequences complimentary thereto.

In another aspect, the invention provides an antibody microarray for use in the methods of the invention, which microarray includes a substrate presenting antibodies capable of binding to a product of expression of a gene the expression of which is upregulated or downregulated when associated with a susceptibility or protective polymorphism as described herein.

In a further aspect the present invention provides a method of treating a subject having an increased risk of developing COPD, emphysema, or both COPD and emphysema comprising the step of replicating, genotypically or phenotypically, the presence and/or functional effect of a protective polymorphism in said subject.

In yet a further aspect, the present invention provides a method of treating a subject having an increased risk of developing COPD, emphysema, or both COPD and emphysema, said subject having a detectable susceptibility polymorphism which either upregulates or downregulates expression of a gene such that the physiologically active concentration of the expressed gene product is outside a range which is normal for the age and sex of the subject, said method comprising the step of restoring the physiologically active concentration of said product of gene expression to be within a range which is normal for the age and sex of the subject.

In yet a further aspect the present invention provides a method of treating a subject having an increased risk of developing COPD, emphysema, or both COPD and emphysema and for whom the presence of the GG genotype at the −765 C/G polymorphism present in the promoter of the gene encoding COX2 has been determined, said method comprising administering to said subject an agent capable of reducing COX2 activity in said subject.

In one embodiment, said agent is a COX2 inhibitor or a nonsteroidal anti-inflammatory drug (NSAID), preferably said COX2 inhibitor is selected from the group consisting of Celebrex (Celecoxib), Bextra (Valdecoxib), and Vioxx (Rofecoxib).

In a further aspect the present invention provides a method of treating a subject having an increased risk of developing COPD, emphysema, or both COPD and emphysema and for whom the presence of the AA genotype at the 105 C/A polymorphism in the gene encoding IL18 has been determined, said method comprising administering to said subject an agent capable of augmenting IL18 activity in said subject.

In yet a further aspect the present invention provides a method of treating a subject having an increased risk of developing COPD, emphysema, or both COPD and emphysema and for whom the presence of the CC genotype at the −133 G/C polymorphism in the promoter of the gene encoding IL18 has been determined, said method comprising administering to said subject an agent capable of augmenting IL18 activity in said subject.

In still a further aspect the present invention provides a method of treating a subject having an increased risk of developing COPD, emphysema, or both COPD and emphysema and for whom the presence of the 5G5G genotype at the −675 4G/5G polymorphism in the promoter of the gene encoding PAI-1 has been determined, said method comprising administering to said subject an agent capable of augmenting PAI-1 activity in said subject.

In a yet further aspect the present invention provides a method of treating a subject having an increased risk of developing COPD, emphysema, or both COPD and emphysema and for whom the presence of the AA genotype at the 874 A/T polymorphism in the gene encoding IFN-γ has been determined, said method comprising administering to said subject an agent capable of modulating IFN-γ activity in said subject.

In still yet a further aspect the present invention provides a method of treating a subject having an increased risk of developing COPD, emphysema, or both COPD and emphysema and for whom the presence of the CC genotype at the −159 C/T polymorphism in the gene encoding CD-14 has been determined, said method comprising administering to said subject an agent capable of modulating CD-14 and/or IgE activity in said subject.

In yet a further aspect, the present invention provides a method for screening for compounds that modulate the expression and/or activity of a gene, the expression of which is upregulated or downregulated when associated with a susceptibility or protective polymorphism, said method comprising the steps of:
contacting a candidate compound with a cell comprising a susceptibility or protective polymorphism which has been determined to be associated with the upregulation or downregulation of expression of a gene; and
measuring the expression of said gene following contact with said candidate compound,
wherein a change in the level of expression after the contacting step as compared to before the contacting step is indicative of the ability of the compound to modulate the expression and/or activity of said gene.

Preferably, said cell is a human lung cell which has been pre-screened to confirm the presence of said polymorphism.

Preferably, said cell includes a susceptibility polymorphism associated with upregulation of expression of said gene and said screening is for candidate compounds which downregulate expression of said gene.

Alternatively, said cell includes a susceptibility polymorphism associated with downregulation of expression of said gene and said screening is for candidate compounds which upregulate expression of said gene.

In another embodiment, said cell includes a protective polymorphism associated with upregulation of expression of said gene and said screening is for candidate compounds which further upregulate expression of said gene.

Alternatively, said cell includes a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism, said method comprising the steps of:
contacting a candidate compound with a cell comprising a gene, the expression of which is upregulated or downregulated when associated with a susceptibility or protective polymorphism but which in said cell the expression of which is neither upregulated nor downregulated; and
measuring the expression of said gene following contact with said candidate compound, wherein a change in the level of expression after the contacting step as compared to before the contacting step is indicative of the ability of the compound to modulate the expression and/or activity of said gene.

Preferably, said cell is human lung cell which has been pre-screened to confirm the presence, and baseline level of expression, of said gene.

Preferably, expression of the gene is downregulated when associated with a susceptibility polymorphism and said screening is for candidate compounds which in said cell, upregulate expression of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject at risk of developing or suffering from COPD, emphysema, or both COPD and emphysema to a prophylactic or therapeutic treatment, which treatment involves restoring the physiologically active concentration of a product of gene expression to be within a range which is normal for the age and sex of the subject, which method includes detecting in said subject the presence or absence of a susceptibility polymorphism which when present either upregulates or down-regulates expression of said gene such that the physiological active concentration of the expressed gene product is outside said normal range, wherein the detection of the presence of said polymorphism is indicative of the subject likely responding to said treatment.

In a further aspect, the present invention provides a kit for assessing a subject's risk of developing one or more obstructive lung diseases selected from COPD, emphysema, or both COPD and emphysema, said kit comprising a means of analysing a sample from said subject for the presence or absence of one or more polymorphisms disclosed herein.

### BRIEF DESCRIPTION OF THE DRAWINGS

FIG. 1 depicts a graph showing the percentage of people with COPD plotted against the number of protective genetic variants.

FIG. 2 depicts a graph showing the percentage of people with COPD plotted against the number of susceptibility genetic variants.

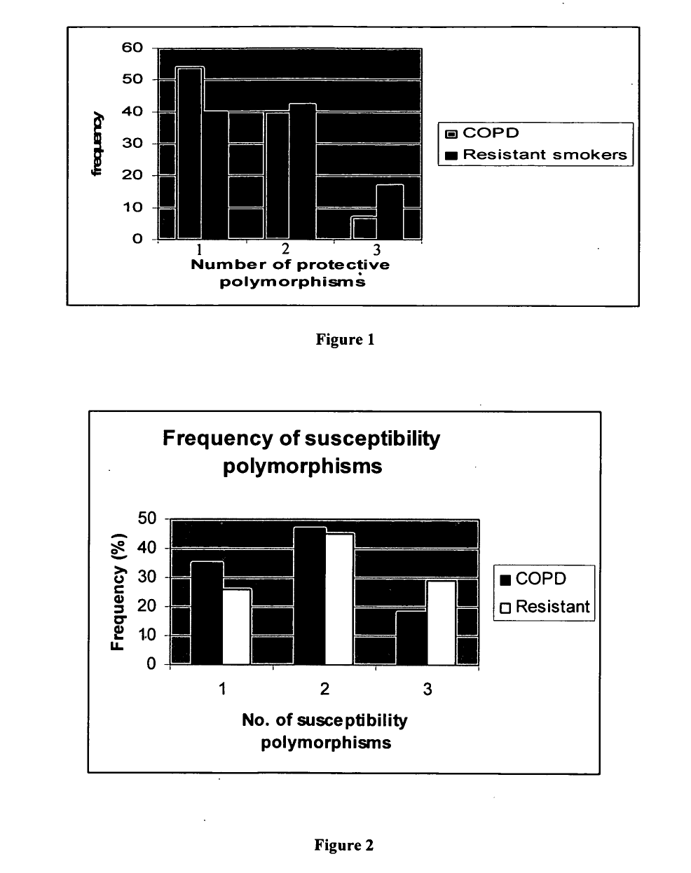

### DETAILED DESCRIPTION OF THE PREFERRED EMBODIMENT

Using case-control studies the frequencies of several genetic variants (polymorphisms) of candidate genes in smokers who have developed COPD, smokers who appear resistant to COPD, and blood donor controls have been compared. The majority of these candidate genes have confirmed (or likely) functional effects on gene expression or protein function. Specifically the frequencies of polymorphisms between blood donor controls, resistant smokers and those with COPD (subdivided into those with early onset and those with normal onset) have been compared. The present invention demonstrates that there are both protective and susceptibility polymorphisms present in selected candidate genes of the patients tested.

Specifically, 17 susceptibility genetic polymorphisms and 19 protective genetic polymorphisms have been identified. These are as follows:

| Gene | Polymorphism | Role |
|---|---|---|
| Cyclo-oxygenase 2 (COX2) | COX2 −765 G/C | CC/CG protective |
| β2-adrenoreceptor (ADBR) | ADBR Arg16Gly | GG susceptibility |
| Interleukin-18 (IL18) | IL18 −133 C/G | CC susceptibility |
| Interleukin-18 (IL18) | IL18 105 A/C | AA susceptibility |
| Plasminogen activator inhibitor 1 (PAI-1) | PAI-1 −675 4G/5G | 5G5G susceptibility |
| Nitric Oxide synthase 3 (NOS3) | NOS3 298 Asp/Glu | TT protective |
| Vitamin D Binding Protein (VDBP) | VDBP Lys 420 Thr | AA/AC protective |
| Vitamin D Binding Protein (VDBP) | VDBP Glu 416 Asp | TT/TG protective |
| Glutathione S Transferase (GSTP-1) | GSTP1 Ile 105 Val | AA protective |
| Interferon γ (IFN-γ) | IFN-γ 874 A/T | AA susceptibility |
| Interleukin-13 (IL13) | IL13 Arg 130 Gln | AA protective |
| Interleukin-13 (IL13) | Il13 −1055 C/T | TT susceptibility |
| α1-antitrypsin (α1-AT) | α1-AT S allele | MS protective |
| Tissue Necrosis Factor α (TNFα) | TNFα +489 G/A | AA/AG susceptibility |
| | | GG protective |
| Tissue Necrosis Factor α (TNFα) | TNFα −308 G/A | GG protective |
| | | AA/AG susceptibility |
| SMAD3 | SMAD3 C89Y AG | AA/AG protective |
| | | GG susceptibility |
| Intracellular adhesion molecule 1 (ICAM1) | ICAM1 E469K A/G | GG susceptibility |
| Caspase (NOD2) | NOD2 Gly 881 Arg G/C | GC/CC susceptibility |
| Mannose binding lectin 2 (MBL2) | MBL2 161 G/A | GG protective |
| Chymase 1 (CMA1) | CMA1 −1903 G/A | AA protective |
| N-Acetyl transferase 2 (NAT2) | NAT2 Arg 197 Gln G/A | AA protective |
| Interleukin 1B (IL1B) | IL1B −511 A/G | GG susceptibility |
| Microsomal epoxide hydrolase (MEH) | MEH Tyr 113 His T/C | TT susceptibility |
| Microsomal epoxide hydrolase (MEH) | MEH His 139 Arg G/A | GG protective |
| 5 Lipo-oxygenase (ALOX5) | ALOX5 −366 G/A | AA/AG protective |
| | | GG susceptibility |
| Heat Shock Protein 70 (HSP 70) | HSP 70 HOM T2437C | CC/CT susceptibility |
| | | TT protective |
| Chloride Channel Calcium-activated 1 (CLCA1) | CLCA1 +13924 T/A | AA susceptibility |
| Monocyte differentiation antigen CD-14 | CD-14 −159 C/T | CC susceptibility |
| Elafin | Elafin Exon 1 +49 C/T | CT/TT protective |
| B2-adrenergic receptor (ADBR) | ADBR Gln 27 Glu C/G | GG protective |
| Matrix metalloproteinase 1 (MMP1) | MMP1 −1607 1G/2G | 1G1G/1G2G protective |

A susceptibility genetic polymorphism is one which, when present, is indicative of an increased risk of developing COPD, emphysema, or both COPD and emphysema. In contrast, a protective genetic polymorphism is one which, when present, is indicative of a reduced risk of developing COPD, emphysema, or both COPD and emphysema.

As used herein, the phrase “risk of developing COPD, emphysema, or both COPD and emphysema” means the likelihood that a subject to whom the risk applies will develop COPD, emphysema, or both COPD and emphysema, and includes predisposition to, and potential onset of the disease. Accordingly, the phrase “increased risk of developing COPD, emphysema, or both COPD and emphysema” means that a subject having such an increased risk possesses a hereditary inclination or tendency to develop COPD, emphysema, or both COPD and emphysema. This does not mean that such a person will actually develop COPD, emphysema, or both COPD and emphysema at any time, merely that he or she has a greater likelihood of developing COPD, emphysema, or both COPD and emphysema compared to the general population of individuals that either does not possess a polymorphism associated with increased COPD, emphysema, or both COPD and emphysema risk, or does possess a polymorphism associated with decreased COPD, emphysema, or both COPD and emphysema risk. Subjects with an increased risk of developing COPD, emphysema, or both COPD and emphysema include those with a predisposition to COPD, emphysema, or both COPD and emphysema, such as a tendency or prediliction regardless of their lung function at the time of assessment, for example, a subject who is genetically inclined to COPD, emphysema, or both COPD and emphysema but who has normal lung function, those at potential risk, including subjects with a tendency to mildly reduced lung function who are likely to go on to suffer COPD, emphysema, or both COPD and emphysema if they keep smoking, and subjects with potential onset of COPD, emphysema, or both COPD and emphysema, who have a tendency to poor lung function on spirometry etc., consistent with COPD at the time of assessment.

Similarly, the phrase “decreased risk of developing COPD, emphysema, or both COPD and emphysema” means that a subject having such a decreased risk possesses an hereditary disinclination or reduced tendency to develop COPD, emphysema, or both COPD and emphysema. This does not mean that such a person will not develop COPD, emphysema, or both COPD and emphysema at any time, merely that he or she has a decreased likelihood of developing COPD, emphysema, or both COPD and emphysema compared to the general population of individuals that either does possess one or more polymorphisms associated with increased COPD, emphysema, or both COPD and emphysema risk, or does not possess a polymorphism associated with decreased COPD, emphysema, or both COPD and emphysema risk.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09gloss.html#p. Accordingly, the term “polymorphisms” is used herein contemplates genetic variations, including single nucleotide substitutions, insertions and deletions of nucleotides, repetitive sequences (such as microsatellites), and the total or partial absence of genes (eg. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and can be in linkage disequilibrium. A haplotype can be identified by patterns of polymorphisms such as single nucleotide polymorphisms, “SNPs.” In some embodiments, the term “single nucleotide polymorphism” or “SNP” in the context of the present invention includes single base nucleotide subsitutions and short deletion and insertion polymorphisms. In other embodiments, SNP refers to a single nucleotide change, such as a substitution, deletion or insertion.

A reduced or increased risk of a subject developing COPD, emphysema, or both COPD and emphysema can be diagnosed by analysing a sample from said subject for the presence of a polymorphism selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1;
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding MMP1 (with reference to the 1G allele only);
and one or more polymorphisms which are in linkage disequilibrium with any one or more of the above group.

These polymorphisms can also be analysed in combinations of two or more, or in combination with other polymorphisms indicative of a subject's risk of developing COPD, emphysema, or both COPD and emphysema, inclusive of the remaining polymorphisms listed above.

Expressly contemplated are combinations of the above polymorphisms with polymorphisms as described in PCT International application PCT/NZ02/00106, published as WO 02/099134, herein incorporated by reference in its entirety.

Assays which involve combinations of polymorphisms, including those amenable to high throughput, such as those utilising microarrays, are preferred.

Statistical analyses, particularly of the combined effects of these polymorphisms, show that the genetic analyses of the present invention can be used to determine the risk quotient of any smoker and in particular to identify smokers at greater risk of developing COPD. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of smokers and non-smokers, as described herein, it is possible to implicate certain proteins in the development of COPD and improve the ability to identify which smokers are at increased risk of developing COPD-related impaired lung function and COPD for predictive purposes.

The present results show for the first time that the minority of smokers who develop COPD, emphysema, or both COPD and emphysema do so because they have one or more of the susceptibility polymorphisms and few or none of the protective polymorphisms defined herein. It is thought that the presence of one or more suscetptible polymorphisms, together with the damaging irritant and oxidant effects of smoking, combine to make this group of smokers highly susceptible to developing COPD, emphysema, or both COPD and emphysema. Additional risk factors, such as familial history, age, weight, pack years, etc., will also have an impact on the risk profile of a subject, and can be assessed in combination with the genetic analyses described herein.

The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present implies the presence of the other. (Reich D E et al; Linkage disequilibrium in the human genome, Nature 2001, 411:199-204.)

Examples of polymorphisms reported to be in linkage disequilibrium are presented herein, and include the Interleukin-18 −133 C/G and 105 A/C polymorphisms, and the Vitamin D binding protein Glu 416 Asp and Lys 420 Thr polymorphisms, as shown below.

| | | rs | Alleles in | LD between | Phenotype in |
|---|---|---|---|---|---|
| Gene | SNPs | numbers | LD | alleles | COPD |
| Interleukin-18 | IL18 −133 | rs360721 | C allele | Strong LD | CC susceptible |
| | C/G | | | | |
| | IL18 105 | rs549908 | G allele | | AA susceptible |
| | A/C | | | | |
| Vitamin D | VDBP Lys | rs4588 | A allele | Strong LD | AA/AC |
| binding protein | 420 Thr | | | | protective |
| | VDBP Glu | rs7041 | T allele | | TT/TG |
| | 416 Asp | | | | protective |

It will be apparent that polymorphsisms in linkage disequilibrium with one or more other polymorphism associated with increased or decreased risk of developing COPD, emphysema, or both COPD and emphysema will also provide utility as biomarkers for risk of developing COPD, emphysema, or both COPD and emphysema. The data presented herein shows that the frequency for SNPs in linkage disequilibrium is very similar. Accordingly, these genetically linked SNPs can be utilized in combined polymorphism analyses to derive a level of risk comparable to that calculated from the original SNP.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented below (at the end of the examples).

The methods of the invention are primarily directed to the detection and identification of the above polymorphisms associated with COPD, which are all single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation (Matsuzaki, H. et al. Genome Res. 14:414-425 (2004); Matsuzaki, H. et al. Nat. Methods 1:109-111 (2004); Sethi, A. A. et al. Clin. Chem. 50(2):443-446 (2004), each of the foregoing is herein incorporated by reference in its entirety). These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607, each of the foregoing is herein incorporated by reference in its entirety). US Application 20050059030 (incorporated herein in its entirety by reference) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

U.S. Application 20050042608 (incorporated by reference herein in its entirety) describes a modification of the method of electrochemical detection of nucleic acid hybridization of Thorp et al. (U.S. Pat. No. 5,871,918, incorporated by reference in its entirety). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP (Ross, P. L. et al. Discrimination of single-nucleotide polymorphisms in human DNA using peptide nucleic acid probes detected by MALDI-TOF mass spectrometry. Anal. Chem. 69, 4197-4202 (1997), herein incorporated by reference in its entirety). This technique varies in how rapidly it can be performed, from a few samples per day to a high throughput of 40,000 SNPs per day, using mass code tags. A preferred example is the use of mass spectrometric determination of a nucleic acid sequence which includes the polymorphisms of the invention, for example, which includes the promoter of the COX2 gene or a complementary sequence. Such mass spectrometric methods are known to those skilled in the art, and the genotyping methods of the invention are amenable to adaptation for the mass spectrometric detection of the polymorphisms of the invention, for example, the COX2 promoter polymorphisms of the invention.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174 (each of the foregoing which is herein incorporated by reference in its entirety).

U.S. Pat. No. 6,821,733 (incorporated herein in its entirety by reference) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots (Sloane, A. J. et al. High throughput peptide mass fingerprinting and protein macroarray analysis using chemical printing strategies. Mol Cell Proteomics 1(7):490-9 (2002), herein incorporated by reference in its entirety). After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 1989 86:2766-2770, (1989), herein incorporated by reference in its entirety) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP (Gasparini, P. et al. Scanning the first part of the neurofibromatosis type 1 gene by RNA-SSCP: identification of three novel mutations and of two new polymorphisms. Hum Genet. 97(4):492-5 (1996), herein incorporated by reference in its entirety), restriction endonuclease fingerprinting-SSCP (Liu, Q. et al. Restriction endonuclease fingerprinting (REF): a sensitive method for screening mutations in long, contiguous segments of DNA. Biotechniques 18(3):470-7 (1995), herein incorporated by reference in its entirety), dideoxy fingerprinting (a hybrid between dideoxy sequencing and SSCP) (Sarkar, G. et al. Dideoxy fingerprinting (ddF): a rapid and efficient screen for the presence of mutations. Genomics 13:441-443 (1992), herein incorporated by reference in its entirety), bi-directional dideoxy fingerprinting (in which the dideoxy termination reaction is performed simultaneously with two opposing primers) (Liu, Q. et al. Bi-directional dideoxy fingerprinting (Bi-ddF): a rapid method for quantitative detection of mutations in genomic regions of 300-600 bp. Hum Mol Genet. 5(1):107-14 (1996), herein incorporated by reference in its entirety), and Fluorescent PCR-SSCP (in which PCR products are internally labelled with multiple fluorescent dyes, can be digested with restriction enzymes, followed by SSCP, and analysed on an automated DNA sequencer able to detect the fluorescent dyes) (Makino, R. et al. F-SSCP: fluorescence-based polymerase chain reaction-single-strand conformation polymorphism (PCR-SSCP) analysis. PCR Methods Appl. 2(1):10-13 (1992), herein incorporated by reference in its entirety).

Other methods which utilise the varying mobility of different nucleic acid structures include Denaturing Gradient Gel Electrophoresis (DGGE) (Cariello, N. F. et al. Resolution of a missense mutant in human genomic DNA by denaturing gradient gel electrophoresis and direct sequencing using in vitro DNA amplification: HPRT Munich. Am J Hum Genet. 42(5):726-34 (1988), herein incorporated by reference in its entirety), Temperature Gradient Gel Electrophoresis (TGGE) (Riesner, D. et al. Temperature-gradient gel electrophoresis for the detection of polymorphic DNA and for quantitative polymerase chain reaction. Electrophoresis. 13:632-6 (1992), herein incorporated by reference in its entirety), and Heteroduplex Analysis (HET) (Keen, J. et al. Rapid detection of single base mismatches as heteroduplexes on Hydrolink gels. Trends Genet. 7(1):5 (1991), herein incorporated by reference in its entirety). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs (Giordano, M. et al. Identification by denaturing high-performance liquid chromatography of numerous polymorphisms in a candidate region for multiple sclerosis susceptibility. Genomics 56(3):247-53 (1999), herein incorporated by reference in its entirety).

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins (Moore, W. et al. Mutation detection in the breast cancer gene BRCA1 using the protein truncation test. Mol Biotechnol. 14(2):89-97 (2000), herein incorporated by reference in its entirety). Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonomous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989 (each of the foregoing is herein incorporated by reference in its entirety).

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes can be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980), herein incorporated by reference in its entirety). Alternatively, the probes can be generated, in whole or in part, enzymatically.

Nucleotide analogs can be incorporated into probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each of the foregoing is herein incorporated by reference in its entirety).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13 (each of the foregoing which is herein incorporated by reference in its entirety).

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention include one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

It will be appreciated that the methods of the invention can be performed in conjunction with an analysis of other risk factors known to be associated with COPD, emphysema, or both COPD and emphysema. Such risk factors include epidemiological risk factors associated with an increased risk of developing COPD, emphysema, or both COPD and emphysema. Such risk factors include, but are not limited to smoking and/or exposure to tobacco smoke, age, sex and familial history. These risk factors can be used to augment an analysis of one or more polymorphisms as herein described when assessing a subject's risk of developing chronic obstructive pulmonary disease (COPD) and/or emphysema.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a SNP allele or genotype is associated with decreased expression of a gene, therapy can involve administration of an agent capable of increasing the expression of said gene, and conversely, where a SNP allele or genotype is associated with increased expression of a gene, therapy can involve administration of an agent capable of decreasing the expression of said gene. Methods useful for the modulation of gene expression are well known in the art. For example, in situations were a SNP allele or genotype is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility SNP allele or genotype is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a SNP allele or genotype is associated with decreased enzyme function, therapy can involve administration of active enzyme or an enzyme analogue to the subject. Similarly, where a SNP allele or genotype is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a beneficial (protective) SNP is associated with upregulation of a particular gene or expression of an enzyme or other protein, therapies can be directed to mimic such upregulation or expression in an individual lacking the resistive genotype, and/or delivery of such enzyme or other protein to such individual Further, when a protective SNP is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to COPD, emphysema, or both COPD and emphysema also has application in the design and/or screening of candidate therapeutics. This is particularly the case where the association between a susceptibility or protective polymorphism is manifested by either an upregulation or downregulation of expression of a gene. In such instances, the effect of a candidate therapeutic on such upregulation or downregulation is readily detectable.

For example, in one embodiment existing human lung organ and cell cultures are screened for SNP genotypes as set forth above. (For information on human lung organ and cell cultures, see, e.g.: Bohinski et al. (1996) Molecular and Cellular Biology 14:5671-5681; Collettsolberg et al. (1996) Pediatric Research 39:504; Hermanns et al. (2004) Laboratory Investigation 84:736-752; Hume et al. (1996) In Vitro Cellular & Developmental Biology-Animal 32:24-29; Leonardi et al. (1995) 38:352-355; Notingher et al. (2003) Biopolymers (Biospectroscopy) 72:230-240; Ohga et al. (1996) Biochemical and Biophysical Research Communications 228:391-396; each of which is hereby incorporated by reference in its entirety.) Cultures representing susceptible and protective genotype groups are selected, together with cultures which are putatively “normal” in terms of the expression of a gene which is either upregulated or downregulated where a protective polymorphism is present.

Samples of such cultures are exposed to a library of candidate therapeutic compounds and screened for any or all of: (a) downregulation of susceptibility genes that are normally upregulated in susceptible genotypes; (b) upregulation of susceptibility genes that are normally downregulated in susceptible genotypes; (c) downregulation of protective genes that are normally downregulated or not expressed (or null forms are expressed) in protective genotypes; and (d) upregulation of protective genes that are normally upregulated in protective genotypes. Compounds are selected for their ability to alter the regulation and/or action of susceptibility genes and/or protective genes in a culture having a susceptible genotype.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

### EXAMPLES

Various embodiments of the invention will now be described in more detail, with reference to non-limiting examples.

### Example 1

### Case Association Study, Cyclo-Oxygenase 2 (COX2)-765 G/C Promoter Polymorphism and α1-Antitrypsin Genotyping Subject Recruitment

Subjects of European descent who had smoked a minimum of fifteen pack years and diagnosed by a physician with chronic obstructive pulmonary disease (COPD) were recruited. Subjects met the following criteria: were over 50 years old and had developed symptoms of breathlessness after 40 years of age, had a Forced expiratory volume in one second (FEV1) as a percentage of predicted <70% and a FEV1/FVC ratio (Forced expiratory volume in one second/Forced vital capacity) of <79% (measured using American Thoracic Society criteria). Two hundred and ninety-four subjects were recruited, of these 58% were male, the mean FEV1/FVC (±95% confidence limits) was 51% (49-53), mean FEV1 as a percentage of predicted was 43 (41-45). Mean age, cigarettes per day and pack year history was 65 yrs (64-66), 24 cigarettes/day (22-25) and 50 pack years (41-55) respectively. Two hundred and seventeen European subjects who had smoked a minimum of twenty pack years and who had never suffered breathlessness and had not been diagnosed with an obstructive lung disease in the past, in particular childhood asthma or chronic obstructive lung disease, were also studied. This control group was recruited through clubs for the elderly and consisted of 63% male, the mean FEV1/FVC (95% CI) was 82% (81-83), mean FEV1 as a percentage of predicted was 96 (95-97). Mean age, cigarettes per day and pack year history was 59 yrs (57-61), 24 cigarettes/day (22-26) and 42 pack years (39-45) respectively. Using a PCR based method (Sandford et al., 1999, Z and S mutations of the α1-antitrypsin gene and the risk of chronic obstructive pulmonary disease. Am J Respir Cell Mol Biol. 20; 287-291, herein incorporated by reference in its entirety), all subjects were genotyped for the α1-antitrypsin mutations (S and Z alleles) and those with the ZZ allele were excluded. The COPD and resistant smoker cohorts were matched for subjects with the MZ genotype (5% in each cohort). 190 European blood donors (smoking status unknown) were recruited consecutively through local blood donor services. Sixty-three percent were men and their mean age was 50 years. On regression analysis, the age difference and pack years difference observed between COPD sufferers and resistant smokers was found not to determine FEV or COPD.

This and the following examples demonstrate that polymorphisms found in greater frequency in COPD patients compared to controls (and/or resistant smokers) can reflect an increased susceptibility to the development of impaired lung function, COPD, and emphysema. Similarly, polymorphisms found in greater frequency in resistant smokers compared to susceptible smokers (COPD patients and/or controls) can reflect a protective role.

TABLE 1A
Summary of characteristics for the COPD, resistant smoker and healthy
blood donors

| Parameter | COPD | | Resistant smokers | | |
|---|---|---|---|---|---|
| Median (IQR) | N = 294 | | N = 217 | | Differences |
| % male | 58% | | 63% | | ns |
| Age (yrs) | 65 | (64-66) | 59 | (57-61) | P < 0.05 |
| Pack years | 50 | (46-53) | 42 | (39-45) | P < 0.05 |
| Cigarettes/day | 24 | (22-25) | 24 | (22-26) | ns |
| FEV1 (L) | 1.6 | (0.7-2.5) | 2.9 | (2.8-3.0) | P < 0.05 |
| FEV1 % predict | 43 | (41-45) | 96% | (95-97) | P < 0.05 |
| FEVI/FVC | 51 | (49-53) | 82 | (81-83) | P < 0.05 |

Means and 95% confidence limits

Genotyping Methods
Cyclo-Oxygenase 2 (COX2)-765 G/C Promoter Polymorphism and α1-Antitrypsin Genotyping

Genomic DNA was extracted from whole blood samples (Maniatis, T., Fritsch, E. F. and Sambrook, J., Molecular Cloning Manual. 1989). The Cyclo-oxygenase 2-765 polymorphism was determined by minor modifications of a previously published method (Papafili A, et al., 2002, incorporated in its entirety herein by reference)). The PCR reaction was carried out in a total volume of 25 ul and contained 20 ng genomic DNA, 500 pmol forward and reverse primers, 0.2 mM dNTPs, 10 mM Tris-HCL (pH 8.4), 150 mM KCl, 1.0 mM MgCl₂ and 1 unit of polymerase (Life Technologies). Cycling times were incubations for 3 min at 95° C. followed by 33 cycles of 50 s at 94° C., 60 s at 66° C. and 60 s at 72° C. A final elongation of 10 min at 72° C. then followed. 4 ul of PCR products were visualised by ultraviolet trans-illumination of a 3% agarose gel stained with ethidium bromide. An aliquot of 3 ul of amplification product was digested for 1 hr with 4 units of AciI (Roche Diagnostics, New Zealand) at 37° C. Digested products were separated on a 2.5% agarose gel run for 2.0 hours at 80 mV with TBE buffer. The products were visualised against a 123 bp ladder using ultraviolet transillumination after ethidium bromide staining. Using a PCR based method referenced above (Sandford et al., 1999), all COPD and resistant smoker subjects were genotyped for the α1-antitrypsin S and Z alleles.

### Example 2

### Elafin +49C/T Polymorphism

Genomic DNA was extracted from whole blood samples (Maniatis, T., Fritsch, E. F. and Sambrook, J., Molecular Cloning Manual. 1989). The Elafin +49 polymorphism was determined by minor modifications of a previously published method [Kuijpers A L A, et al. Clinical Genetics 1998; 54: 96-101.] incorporated in its entirety herein by reference)). The PCR reaction was carried out in a total volume of 25 ul and contained 20 ng genomic DNA, 500 pmol forward and reverse primers, 0.2 mM dNTPs, 10 mM Tris-HCL (pH 8.4), 150 mM KCl, 1.0 mM MgCl₂ and 1 unit of Taq polymerase] (Life Technologies). Cycling times were incubations for 3 min at 95° C. followed by 33 cycles of 50 s at 94° C., 60 s at 66° C. and 60 s at 72° C. A final elongation of 10 min at 72° C. then followed. 4 ul of PCR products were visualised by ultraviolet trans-illumination of a 3% agarose gel stained with ethidium bromide. An aliquot of 3 ul of amplification product was digested for 1 hr with 4 units of Fok 1 (Roche Diagnostics, New Zealand) at 37° C. Digested products were separated on a 2.5% agarose gel run for 2.0 hours at 80 mV with TBE buffer. The products were visualised against a 123 bp ladder using ultraviolet transillumination after ethidium bromide staining.

### Example 3

### Genotyping of the −1607 1G2G Polymorphism of the Matrix Metalloproteinase 1 Gene

Genomic DNA was extracted using standard phenol and chloroform methods. Cohorts of patients and controls were configured in to 96-well PCR format containing strategic negative controls. The assay primers, PCR conditions and RFLP assays details have been previously described (Dunleavey, L. et al. Rapid genotype analysis of the matrix metalloproteinase-1 gene 1G/2G polymorphism that is associated with risk of cancer. Matrix Biol. 19(2):175-7 (2000), herein incorporated by reference in its entirety). Genotyping was done using minor modifications of the above protocol optimised for our own laboratory conditions. The PCR reactions were amplified in MJ Research thermocyclers in a total volume of 25 μl and contained 80 ng genomic DNA, 100 ng forward and reverse primers, 200 mM dNTPs, 20 mM Tris-HCL (pH 8.4), 50 mM KCl, 1.5 mM MgCl₂ and 1.0 unit of Taq polymerase (Qiagen). Forward and reverse prime sequences were 3′ TCG TGA GAA TGT CTT CCC ATT-3′ [SEQ ID NO. 1] and 5′TCT TGG ATT GAT TTG AGA TAA GTG AAA TC-3′ [SEQ ID NO. 2]. Cycling conditions consisted of 94 C 60 s, 55 C 30 s, 72 C 30 s for 35 cycles with an extended last extension of 3 min. Aliquots of amplification product were digested for 4 hrs with 6 Units of the restriction enzymes XmnI (Roche Diagnostics, New Zealand) at designated temperature conditions. Digested products were separated on 6% polyacrylamide gel. The products were visualised by ultraviolet transillumination following ethidium bromide staining and migration compared against a 1 Kb plus ladder standard (Invitrogen). Genotypes were recorded in data spreadsheets and statistical analysis performed.

### Example 4

### Other Polymorphism Genotyping

Genomic DNA was extracted from whole blood samples (Maniatis, T., Fritsch, E. F. and Sambrook, J., Molecular Cloning Manual. 1989). Purified genomic DNA was aliquoted (10 ng/ul concentration) into 96 well plates and genotyped on a Sequenom™ system (Sequenom™ Autoflex Mass Spectrometer and Samsung 24 pin nanodispenser) using the following sequences, amplification conditions and methods.

The following conditions were used for the PCR multiplex reaction: final concentrations were for 10× Buffer 15 mM MgCl₂ 1.25×, 25 mM MgCl₂ 1.625 mM, dNTP mix 25 mM 500 uM, primers 4 uM 100 nM, Taq polymerase (Qiagen hot start) 0.15U/reaction, Genomic DNA 10 ng/ul. Cycling times were 95° C. for 15 min, (5° C. for 15 s, 56° C. 30 s, 72° C. 30 s for 45 cycles with a prolonged extension time of 3 min to finish. Shrimp alkaline phosphotase (SAP) treatment was used (2 ul to 5 ul per PCR reaction) incubated at 35° C. for 30 min and extension reaction (add 2 ul to 7 ul after SAP treatment) with the following volumes per reaction of: water, 0.76 ul; hME 10× termination buffer, 0.2 ul; hME primer (10 uM), 1 ul; MassEXTEND enzyme, 0.04 ul.

TABLE 1B

Sequenom conditions for the polymorphisms genotyping-1

| | | | | |
|---|---|---|---|---|
| SNP_ID | TERM | WELL | 2nd-PCRP | 1st-PCRP |

| | | | | |
|---|---|---|---|---|
| Vitamin | ACT | W1 | ACGTTGGATGGCTTGTTAACCAGCTTTGCC | ACGTTGGATGTTTTTCAGACTGGCAGAGCG |
| DBP-420 | | | [SEQ. ID. NO. 3] | [SEQ. ID. NO. 4] |
| Vitamin | ACT | W1 | ACGTTGGATGTTTTTCAGACTGGCAGAGCG | ACGTTGGATGGCTTGTTAACCAGCTTTGCC |
| DBP-416 | | | [SEQ. ID. NO. 5] | [SEQ. ID. NO. 6] |
| IL13 C- | ACT | W2 | ACGTTGGATGCATGTCGCCTTTTCCTGCTC | ACGTTGGATGCAACACCCAACAGGCAAATG |
| 1055T | | | [SEQ. ID. NO. 7] | [SEQ. ID. NO. 8] |
| GSTP1- | ACT | W2 | ACGTTGGATGTGGTGGACATGGTGAATGAC | ACGTTGGATGTGGTGCAGATGCTCACATAG |
| 105 | | | [SEQ. ID. NO. 9] | [SEQ. ID. NO. 10] |
| PAI1 G- | ACT | W2 | ACGTTGGATGCACAGAGAGAGTCTGGACAC | ACGTTGGATGCTCTTGGTCTTTCCCTCATC |
| 675G | | | [SEQ. ID. NO. 11] | [SEQ. ID. NO. 12] |
| NOS3-298 | ACT | W3 | ACGTTGGATGACAGCTCTGCATTCAGCACG | ACGTTGGATGAGTCAATCCCTTTGGTGCTC |
| | | | [SEQ. ID. NO. 13] | [SEQ. ID. NO. 14] |
| IL13- | ACT | W3 | ACGTTGGATGGTTTTCCAGCTTGCATGTCC | ACGTTGGATGCAATAGTCAGGTCCTGTCTC |
| Arg130Gln | | | [SEQ. ID. NO. 15] | [SEQ. ID. NO. 16] |
| ADRB2- | ACT | W3 | ACGTTGGATGGAACGGCAGCGCCTTCTTG | ACGTTGGATGACTTGGCAATGGCTGTGATG |
| Arg16Gly | | | [SEQ. ID. NO. 17] | [SEQ. ID. NO. 18] |
| IFNG- | CGT | W5 | ACGTTGGATGCAGACATTCACAATTGATTT | ACGTTGGATGGATAGTTCCAAACATGTGCG |
| A874T | | | [SEQ. ID. NO. 19] | [SEQ. ID. NO. 20] |
| IL18-C- | ACT | W6 | ACGTTGGATGGGGTATTCATAAGCTGAAAC | ACGTTGGATGCCTTCAAGTTCAGTGGTCAG |
| 133G | | | [SEQ. ID. NO. 21] | [SEQ. ID. NO. 22] |
| IL18- | ACT | W8 | ACGTTGGATGGGTCAATGAAGAGAACTTGG | ACGTTGGATGAATGTTTATTGTAGAAAACC |
| A105C | | | [SEQ. ID. NO. 23] | [SEQ. ID. NO. 24] |

Sequenom conditions for the polymorphisms genotyping-2

| | | | | | | | |
|---|---|---|---|---|---|---|---|
| SNP_ID | AMP_LEN | UP_CONF | MP_CONF | Tm (NN) | PcGC | PWARN | UEP_DIR |

| | | | | | | | |
|---|---|---|---|---|---|---|---|
| Vitamin DBP-420 | 99 | 99.7 | 99.7 | 46.2 | 53.3 | ML | R |
| Vitamin DBP-416 | 99 | 99.7 | 99.7 | 45.5 | 33.3 | M | F |
| IL13 C-1055T | 112 | 97.5 | 80 | 48.2 | 60 | L | R |
| GSTP1-105 | 107 | 99.4 | 80 | 49.9 | 52.9 | | F |
| PAI1 G-675G | 109 | 97.9 | 80 | 59.3 | 66.7 | g | F |
| NOS3-298 | 186 | 98.1 | 65 | 61.2 | 63.2 | | F |
| IL13-Arg130Gln | 171 | 99.3 | 65 | 55.1 | 47.6 | | F |
| ADRB2-Arg16Gly | 187 | 88.2 | 65 | 65.1 | 58.3 | | F |
| IFNG-A874T | 112 | 75.3 | 81.2 | 45.6 | 27.3 | | F |
| IL18-C-133G | 112 | 93.5 | 74.3 | 41.8 | 46.7 | L | F |
| IL18-A105C | 121 | 67.2 | 74.3 | 48.9 | 40 | | R |

Sequenom conditions for the polymorphisms genotyping-3

| | | | | |
|---|---|---|---|---|
| SNP_ID | UEP_MASS | UEP_SEQ | EXT1_CALL | EXT1_MASS |

| | | | | |
|---|---|---|---|---|
| Vitamin DBP-420 | 4518.9 | AGCTTTGCCAGTTCC [SEQ ID NO. 25] | A | 4807.1 |
| Vitamin DBP-416 | 5524.6 | AAAAGCAAAATTGCCTGA [SEQ ID NO. 26] | T | 5812.8 |
| IL13 C-1055T | 4405.9 | TCCTGCTCTTCCCTC [SEQ ID NO. 27] | T | 4703.1 |
| GSTP1-105 | 5099.3 | ACCTCCGCTGCAAATAC [SEQ ID NO. 28] | A | 5396.5 |
| PAI1 G-675G | 5620.6 | GAGTCTGGACACGTGGGG [SEQ ID NO. 29] | DEL | 5917.9 |
| NOS3-298 | 5813.8 | TGCTGCAGGCCCCAGATGA [SEQ ID NO. 30] | T | 6102 |
| IL13-Arg130Gln | 6470.2 | AGAAACTTTTTCGCGAGGGAC [SEQ ID NO. 31] | A | 6767.4 |
| ADRB2-Arg16Gly | 7264.7 | AGCGCCTTCTTGCTGGCACCCAAT [SEQ ID NO. 32] | A | 7561.9 |
| IFNG-A874T | 6639.4 | TCTTACAACACAAAATCAAATC [SEQ ID NO. 33] | T | 6927.6 |
| IL18-C-133G | 4592 | AGCTGAAACTTCTGG [SEQ ID NO. 34] | C | 4865.2 |
| IL18-A105C | 6085 | TCAAGCTTGCCAAAGTAATC [SEQ ID NO. 35] | A | 6373.2 |

Sequenom conditions for the polymorphisms genotyping-4

1st

| | | | | | |
|---|---|---|---|---|---|
| SNP_ID | EXT1_SEQ | EXT2_CALL | EXT2_MASS | EXT2_SEQ | PAUSE |

| | | | | | |
|---|---|---|---|---|---|
| VitaminDBP-420 | AGCTTTGCCAGTTCCT | C | 5136.4 | AGCTTTGCCAGTTCCGT | 4848.2 |
| | [SEQ. ID. NO. 36] | | | [SEQ. ID. NO. 37] | |
| VitaminDBP-416 | AAAAGCAAAATTGCCTGAT | G | 6456.2 | AAAAGCAAAATTGCCTGAGGC | 5853.9 |
| | [SEQ. ID. NO. 38] | | | [SEQ. ID. NO. 39] | |
| IL13C-1055T | TCCTGCTCTTCCCTCA | C | 5023.3 | TCCTGCTCTTCCCTCGT | 4735.1 |
| | [SEQ. ID. NO. 40] | | | [SEQ. ID. NO. 41] | |
| GSTP1-105 | ACCTCCGCTGCAAATACA | G | 5716.7 | ACCTCCGCTGCAAATACGT | 5428.5 |
| | [SEQ. ID. NO. 42] | | | [SEQ. ID. NO. 43] | |
| PAI1G-675G | GAGTCTGGACACGTGGGGA | G | 6247.1 | GAGTCTGGACACGTGGGGGA | 5949.9 |
| | [SEQ. ID. NO. 44] | | | [SEQ. ID. NO. 45] | |
| NOS3-298 | TGCTGCAGGCCCCAGATGAT | G | 6416.2 | TGCTGCAGGCCCCAGATGAGC | 6143 |
| | [SEQ. ID. NO. 46] | | | [SEQ. ID. NO. 47] | |
| IL13-Arg130Gln | AGAAACTTTTTCGCGAGGGACA | G | 7416.8 | AGAAACTTTTTCGCGAGGGACGGT | 6799.4 |
| | [SEQ. ID. NO. 48] | | | [SEQ. ID. NO. 49] | |
| ADRB2-Arg16Gly | AGCGCCTTCTTGCTGGCACCCAATA | G | 8220.3 | AGCGCCTTCTTGCTGGCACCCAATGGA | 7593.9 |
| | [SEQ. ID. NO. 50] | | | [SEQ. ID. NO. 51] | |
| IFNG-A874T | TCTTACAACACAAAATCAAATCT | A | 7225.8 | TCTTACAACACAAAATCAAATCAC | 6952.6 |
| | [SEQ. ID. NO. 52] | | | [SEQ. ID. NO. 53] | |
| IL18-C-133G | AGCTGAAACTTCTGGC | G | 5218.4 | AGCTGAAACTTCTGGGA | 4921.2 |
| | [SEQ. ID. NO. 54] | | | [SEQ. ID. NO. 55] | |
| IL18-A105C | TCAAGCTTGCCAAAGTAATCT | C | 7040.6 | TCAAGCTTGCCAAAGTAATCGGA | 6414.2 |
| | [SEQ. ID. NO. 56] | | | [SEQ. ID. NO. 57] | |

Sequenom conditions for the polymorphisms genotyping-5

| | | |
|---|---|---|
| SNP_ID | 2nd-PCRP | 1st-PCRP |

| | | |
|---|---|---|
| Lipoxygenase5-366G/A | ACGTTGGATGGAAGTCAGAGATGATGGCAG | ACGTTGGATGATGAATCCTGGACCCAAGAC |
| | [SEQ. ID. NO. 58] | [SEQ. ID. NO. 59] |
| TNFalpha + 489G/A | ACGTTGGATGGAAAGATGTGCGCTGATAGG | ACGTTGGATGGCCACATCTCTTTCTGCATC |
| | [SEQ. ID. NO. 60] | [SEQ. ID. NO. 61] |
| SMAD3C89Y | ACGTTGGATGTTGCAGGTGTCCCATCGGAA | ACGTTGGATGTAGCTCGTGGTGGCTGTGCA |
| | [SEQ. ID. NO. 62] | [SEQ. ID. NO. 63] |
| CaspaseGly881ArgG/C | ACGTTGGATGGTGATCACCCAAGGCTTCAG | ACGTTGGATGGTCTGTTGACTCTTTTGGCC |
| | [SEQ. ID. NO. 64] | [SEQ. ID. NO. 65] |
| MBL2 + 161G/A | ACGTTGGATGGTAGCTCTCCAGGCATCAAC | ACGTTGGATGGTACCTGGTTCCCCCTTTTC |
| | [SEQ. ID. NO. 66] | [SEQ. ID. NO. 67] |
| HSP70-HOM2437T/C | ACGTTGGATGTGATCTTGTTCACCTTGCCG | ACGTTGGATGAGATCGAGGTGACGTTTGAC |
| | [SEQ. ID. NO. 68] | [SEQ. ID. NO. 69] |
| CD14-159C/T | ACGTTGGATGAGACACAGAACCCTAGATGC | ACGTTGGATGGCAATGAAGGATGTTTCAGG |
| | [SEQ. ID. NO. 70] | [SEQ. ID. NO. 71] |
| Chymase1-1903G/A | ACGTTGGATGTAAGACAGCTCCACAGCATC | ACGTTGGATGTTCCATTTCCTCACCCTCAG |
| | [SEQ. ID. NO. 72] | [SEQ. ID. NO. 73] |
| TNFalpha-308G/A | ACGTTGGATGGATTTGTGTGTAGGACCCTG | ACGTTGGATGGGTCCCCAAAAGAAATGGAG |
| | [SEQ. ID. NO. 74] | [SEQ. ID. NO. 75] |
| CLCA1 + 13924T/A | ACGTTGGATGGGATTGGAGAACAAACTCAC | ACGTTGGATGGGCAGCTGTTACACCAAAAG |
| | [SEQ. ID. NO. 76] | [SEQ. ID. NO. 77] |
| MEHTyr113HisT/C | ACGTTGGATGCTGGCGTTTTGCAAACATAC | ACGTTGGATGTTGACTGGAAGAAGCAGGTG |
| | [SEQ. ID. NO. 78] | [SEQ. ID. NO. 79] |
| NAT2Arg197GlnG/A | ACGTTGGATGCCTGCCAAAGAAGAAACACC | ACGTTGGATGACGTCTGCAGGTATGTATTC |
| | [SEQ. ID. NO. 80] | [SEQ. ID. NO. 81] |
| MEHHis139ArgG/A | ACGTTGGATGACTTCATCCACGTGAAGCCC | ACGTTGGATGAAACTCGTAGAAAGAGCCGG |
| | [SEQ. ID. NO. 82] | [SEQ. ID. NO. 83] |
| IL-1B-511A/G | ACGTTGGATGATTTTCTCCTCAGAGGCTCC | ACGTTGGATGTGTCTGTATTGAGGGTGTGG |
| | [SEQ. ID. NO. 84] | [SEQ. ID. NO. 85] |
| ADRB2Gln27GluC/G | ACGTTGGATGTTGCTGGCACCCAATGGAAG | ACGTTGGATGATGAGAGACATGACGATGCC |
| | [SEQ. ID. NO. 86] | [SEQ. ID. NO. 87] |
| ICAM1E469KA/G | ACGTTGGATGACTCACAGAGCACATTCACG | ACGTTGGATGTGTCACTCGAGATCTTGAGG |
| | [SEQ. ID. NO. 88] | [SEQ. ID. NO. 89] |

Sequenom conditions for the polymorphisms genotyping-6

| | | | | | | |
|---|---|---|---|---|---|---|
| SNP_ID | AMP_LEN | UP_CONF | MP_CONF | Tm (NN) | PcGC | UEP_DIR |

| | | | | | | |
|---|---|---|---|---|---|---|
| Lipoxygenase5-366G/A | 104 | 99.6 | 73.4 | 59 | 70.6 | F |
| TNFalpha + 489G/A | 96 | 99.6 | 73.4 | 45.5 | 38.9 | F |
| SMAD3C89Y | 107 | 87.3 | 71.7 | 45.7 | 47.1 | F |
| CaspaseGly881ArgG/C | 111 | 97.2 | 81 | 52.9 | 58.8 | R |
| MBL2 + 161G/A | 99 | 96.8 | 81 | 50.3 | 52.9 | F |
| HSP70-HOM2437T/C | 107 | 99.3 | 81 | 62.2 | 65 | R |
| CD14-159C/T | 92 | 98 | 76.7 | 53.3 | 50 | F |
| Chymase1-1903G/A | 105 | 99.6 | 76.7 | 53.6 | 39.1 | R |
| TNFalpha-308G/A | 100 | 99.7 | 81.6 | 59.9 | 70.6 | R |
| CLCA1 + 13924T/A | 101 | 98 | 98 | 45.3 | 36.8 | R |
| MEHTyr113HisT/C | 103 | 97.7 | 82.2 | 48.7 | 42.1 | R |
| NAT2Arg197GlnG/A | 115 | 97.4 | 70 | 48.5 | 36.4 | F |
| MEHHis139ArgG/A | 115 | 96.7 | 77.8 | 66 | 82.4 | F |
| IL-1B-511A/G | 111 | 99.2 | 83 | 46 | 47.1 | R |
| ADRB2Gln27GluC/G | 118 | 96.6 | 80 | 52.2 | 66.7 | F |
| ICAM1E469KA/G | 115 | 98.8 | 95.8 | 51.5 | 52.9 | R |

Sequenom conditions for the polymorphisms genotyping-7

| | | | | |
|---|---|---|---|---|
| SNP_ID | UEP_MASS | UEP_SEQ | EXT1_CALL | EXT1_MASS |

| | | | | |
|---|---|---|---|---|
| Lipoxygenase5-366G/A | 5209.4 | GTGCCTGTGCTGGGCTC [SEQ. ID. NO. 90] | A | 5506.6 |
| TNFalpha + 489G/A | 5638.7 | GGATGGAGAGAAAAAAAC [SEQ. ID. NO. 91] | A | 5935.9 |
| SMAD3C89Y | 5056.3 | CCCTCATGTCATCTACT [SEQ. ID. NO. 92] | A | 5353.5 |
| CaspaseGly881ArgG/C | 5097.3 | GTCACCCACTCTGTTGC [SEQ. ID. NO. 93] | G | 5370.5 |
| MBL2 + 161G/A | 5299.5 | CAAAGATGGGCGTGATG [SEQ. ID. NO. 94] | A | 5596.7 |
| HSP70-HOM2437T/C | 6026.9 | CCTTGCCGGTGCTCTTGTCC [SEQ. ID. NO. 95] | T | 6324.1 |
| CD14-159C/T | 6068 | CAGAATCCTTCCTGTTACGG [SEQ. ID. NO. 96] | C | 6341.1 |
| Chymase1-1903G/A | 6973.6 | TCCACCAAGACTTAAGTTTTGCT [SEQ. ID. NO. 97] | G | 7246.7 |
| TNFalpha-308G/A | 5156.4 | GAGGCTGAACCCCGTCC [SEQ. ID. NO. 98] | G | 5429.5 |
| CLCA1 + 13924T/A | 5759.8 | CTTTTTCATAGAGTCCTGT [SEQ. ID. NO. 99] | A | 6048 |
| MEHTyr113HisT/C | 5913.9 | TTAGTCTTGAAGTGAGGGT [SEQ. ID. NO. 100] | T | 6211.1 |
| NAT2Arg197GlnG/A | 6635.3 | TACTTATTTACGCTTGAACCTC [SEQ. ID. NO. 101] | A | 6932.5 |
| MEHHis139ArgG/A | 5117.3 | CCAGCTGCCCGCAGGCC [SEQ. ID. NO. 102] | A | 5414.5 |
| IL-1B-511A/G | 5203.4 | AATTGACAGAGAGCTCC [SEQ. ID. NO. 103] | G | 5476.6 |
| ADRB2Gln27GluC/G | 4547 | CACGACGTCACGCAG [SEQ. ID. NO. 104] | C | 4820.2 |
| ICAM1E469KA/G | 5090.3 | CACATTCACGGTCACCT [SEQ. ID. NO. 105] | G | 5363.5 |

Sequenom conditions for the polymorphisms genotyping-8

| | | |
|---|---|---|
| EXT2 | EXT2 | 1st |

| | | | | | |
|---|---|---|---|---|---|
| SNP_ID | EXT1_SEQ | CALL | MASS | EXT2_SEQ | PAUSE |

| | | | | | |
|---|---|---|---|---|---|
| Lipoxygenase5-366G/A | GTGCCTGTGCTGGGCTCA | G | 5826.8 | GTGCCTGTGCTGGGCTCGT | 5538.6 |
| | [SEQ. ID. NO. 106] | | | [SEQ. ID. NO. 107] | |
| TNFalpha + 489G/A | GGATGGAGAGAAAAAAACA | G | 6256.1 | GGATGGAGAGAAAAAAACGT | 5967.9 |
| | [SEQ. ID. NO. 108] | | | [SEQ. ID. NO. 109] | |
| SMAD3C89Y | CCCTCATGTCATCTACTA | G | 5658.7 | CCCTCATGTCATCTACTGC | 5385.5 |
| | [SEQ. ID. NO. 110] | | | [SEQ. ID. NO. 111] | |
| CaspaseGly881ArgG/C | GTCACCCACTCTGTTGCC | C | 5699.7 | GTCACCCACTCTGTTGCGC | 5426.5 |
| | [SEQ. ID. NO. 112] | | | [SEQ. ID. NO. 113] | |
| MBL2 + 161G/A | CAAAGATGGGCGTGATGA | G | 5901.9 | CAAAGATGGGCGTGATGGC | 5628.7 |
| | [SEQ. ID. NO. 114] | | | [SEQ. ID. NO. 115] | |
| HSP70-HOM2437T/C | CCTTGCCGGTGCTCTTGTCCA | C | 6644.3 | CCTTGCCGGTGCTCTTGTCCGT | 6356.1 |
| | [SEQ. ID. NO. 116] | | | [SEQ. ID. NO. 117] | |
| CD14-159C/T | CAGAATCCTTCCTGTTACGGC | T | 6645.3 | CAGAATCCTTCCTGTTACGGTC | 6372.2 |
| | [SEQ. ID. NO. 118] | | | [SEQ. ID. NO. 119] | |
| Chymase1-1903G/A | TCCACCAAGACTTAAGTTTTGCTC | A | 7550.9 | TCCACCAAGACTTAAGTTTTGCTTC | 7277.8 |
| | [SEQ. ID. NO. 120] | | | [SEQ. ID. NO. 121] | |
| TNFalpha-308G/A | GAGGCTGAACCCCGTCCC | A | 5733.7 | GAGGCTGAACCCCGTCCTC | 5460.6 |
| | [SEQ. ID. NO. 122] | | | [SEQ. ID. NO. 123] | |
| CLCA1 + 13924T/A | CTTTTTCATAGAGTCCTGTT | T | 6659.4 | CTTTTTCATAGAGTCCTGTAAC | 6073 |
| | [SEQ. ID. NO. 124] | | | [SEQ. ID. NO. 125] | |
| MEHTyr113HisT/C | TTAGTCTTGAAGTGAGGGTA | C | 6531.3 | TTAGTCTTGAAGTGAGGGTGT | 6243.1 |
| | [SEQ. ID. NO. 126] | | | [SEQ. ID. NO. 127] | |
| NAT2Arg197GlnG/A | TACTTATTTACGCTTGAACCTCA | G | 7261.8 | TACTTATTTACGCTTGAACCTCGA | 6964.5 |
| | [SEQ. ID. NO. 128] | | | [SEQ. ID. NO. 129] | |
| MEHHis139ArgG/A | CCAGCTGCCCGCAGGCCA | G | 5734.7 | CCAGCTGCCCGCAGGCCGT | 5446.5 |
| | [SEQ. ID. NO. 130] | | | [SEQ. ID. NO. 131] | |
| IL-1B-511A/G | AATTGACAGAGAGCTCCC | A | S820.8 | AATTGACAGAGAGCTCCTG | 5507.6 |
| | [SEQ. ID. NO. 132] | | | [SEQ. ID. NO. 133] | |
| ADRB2Gln27GluC/G | CACGACGTCACGCAGC | G | 5173.4 | CACGACGTCACGCAGGA | 4876.2 |
| | [SEQ. ID. NO. 134] | | | [SEQ. ID. NO. 135] | |
| ICAM1E469KA/G | CACATTCACGGTCACCTC | A | 5707.7 | CACATTCACGGTCACCTTG | 5394.5 |
| | [SEQ. ID. NO. 136] | | | [SEQ. ID. NO. 137] | |

Results

The following examples demonstrate how to identify if a particular polymorphism allele, genotype frequency, or both, is indicative of a protective role or a susceptibility role. The conditions used for identifying the alleles and genotypes, as well as identifying and characterizing control, susceptible, and resistant groups, are outlined in Table 1B above and in the previous examples.

### Example 5

### Cyclo-Oxygenase 2-765 G/C Polymorphism Allele and Genotype Frequency in the COPD Patients, Resistant Smokers and Controls

The genotype frequency for the above allele was determined in COPD patients (which can serve as an emphysema model), resistant smokers, and controls. The frequencies are shown in the following table.

TABLE 1C
Cyclo-oxygenase 2 −765 G/C polymorphism allele and genotype
frequency in the COPD patients, resistant smokers and controls.

| | 1. Allele* | | 2. Genotype | | |
|---|---|---|---|---|---|
| Frequency | C | G | CC | CG | GG |
| Controls n = 94 (%) | 27 | 161 | 3 | 21 | 70 |
| | (14%) | (86%) | (3%) | (22%) | (75%) |
| COPD n = 202 (%) | 59 | 345 | 6 | 47 | 149¹ |
| | (15%) | (85%) | (3%) | (23%) | (74%) |
| Resistant n = 172 | 85² | 259 | 14 | 57¹ | 101¹ |
| (%) | (25%) | (75%) | (8%) | (33%) | (59%) |

*number of chromosomes (2n)Genotype

A mathematical analysis of the data in the table indicated that:
1. Genotype. CC/CG vs GG for resistant vs COPD, Odds ratio (OR)=1.98, 95% confidence limits 1.3-3.1, χ² (Yates corrected)=8.82, p=0.003, CC/CG=protective for COPD and
2. Allele. C vs G for resistant vs COPD, Odds ratio (OR)=1.92, 95% confidence limits 1.3-2.8, χ² (Yates corrected)=11.56, p<0.001, C=protective for COPD.
   Thus, for the −765 C/G promoter polymorphisms of cyclo-oxygenase 2 gene, the C allele and CC/CG genotype were found to be significantly greater in the resistant smoker cohort compared to the COPD cohort (OR=1.92, P<0.001 and OR=1.98, P=0.003) consistent with a protective role. The greater frequency compared to the blood donor cohort also suggests that the C allele (CC genotype) is over represented in the resistant group (see Table 1C).

### Example 6

### Beta2-Adrenoreceptor Arg 16 Gly Polymorphism Allele and Genotype Frequency in the COPD Patients, Resistant Smokers and Controls

The genotype frequency for the above allele was determined in COPD patients (which can serve as an emphysema model), resistant smokers, and controls. The frequencies are shown in the following table.

TABLE 2
Beta2-adrenoreceptor Arg 16 Gly polymorphism allele and genotype
frequency in the COPD patients, resistant smokers and controls.

| | 3. Allele* | | 4. Genotype | | |
|---|---|---|---|---|---|
| Frequency | A | G | AA | AG | GG |
| Controls n = 182 (%) | 152 | 212 | 26 | 100 | 56 |
| | (42%) | (58%) | (14%) | (55%) | (31%) |
| COPD n = 236 (%) | 164 | 308 | 34 | 96 | 106¹ |
| | (34%) | (66%) | (14%) | (41%) | (45%) |
| Resistant n = 190 | 135 | 245 | 34 | 67 | 89² |
| (%) | (36%) | (64%) | (18%) | (35%) | (47%) |

*number of chromosomes (2n)

A mathematical analysis of the data in the table indicated that:
1. Genotype. GG vs AG/AA for COPD vs controls, Odds ratio (OR)=1.83, 95% confidence limits 1.2-2.8, χ² (Yates corrected)=8.1, p=0.004,
GG=susceptible to COPD (depending on the presence of other snps)
2. Genotype. GG vs AG/AA for resistant vs controls, Odds ratio (OR)=1.98, 95% confidence limits 1.3-3.1, χ² (Yates corrected)=9.43, p=0.002
GG=protective for COPD (depending on the presence of other snps)
   Thus, for the Arg16Gly polymorphism of the β2 adrenergic receptor gene, the GG genotype was found to be significantly greater in the COPD cohort compared to the controls (OR=1.83, P=0.004) suggesting a possible susceptibility to smoking associated with this genotype. Although the GG genotype is also over-represented in the resistant cohort its effects can be overshadowed by protective polymorphisms (see Table 2).

### Example 7

### Interleukin 18 105 A/C Polymorphism Allele and Genotype Frequency in the COPD Patients, Resistant Smokers and Controls

The genotype frequency for the above allele was determined in COPD patients (which can serve as an emphysema model), resistant smokers, and controls. The frequencies are shown in the following table.

TABLE 3a
Interleukin 18 105 A/C polymorphism allele and genotype
frequency in the COPD patients, resistant smokers and controls.

| | 5. Allele* | | 6. Genotype | | |
|---|---|---|---|---|---|
| Frequency | C | A | CC | AC | AA |
| Controls n = 184 (%) | 118 | 250 | 22 | 74 | 88 |
| | (32%) | (68%) | (12%) | (40%) | (48%) |
| COPD n = 240 (%) | 122 | 377² | 21 | 80 | 139^{1,3} |
| | (25%) | (75%) | (9%) | (33%) | (58%) |
| Resistant n = 196 (%) | 113 | 277 | 16 | 81 | 99 |
| | (29%) | (71%) | (8%) | (41%) | (50%) |

*number of chromosomes (2n)

A mathematical analysis of the data in the table indicated that:
1. Genotype. AA vs AC/CC for COPD vs controls, Odds ratio (OR)=1.50, 95% confidence limits 1.0-2.3, χ² (Yates uncorrected)=4.26, p=0.04,
AA=susceptible to COPD
2. Allele. A vs C for COPD vs control, Odds ratio (OR)=1.46, 95% confidence limits 1.1-2.0, χ² (Yates corrected)=5.76, p=0.02
3. Genotype. AA vs AC/CC for COPD vs resistant, Odds ratio (OR)=1.35, 95% confidence limits 0.9-2.0, χ² (Yates uncorrected)=2.39, p=0.12 (trend) AA=susceptible to COPD
   Thus, for the 105 C/A polymorphism of the IL18 gene, the A allele and AA genotype were found to be significantly greater in the COPD cohort compared to the controls (OR=1.46, P=0.02 and OR=1.50, P=0.04 respectively) consistent with a susceptibility role. The AA genotype was also greater in the COPD cohort compared with resistant smokers (OR 1.4, P=0.12) a trend consistent with a susceptibility role (see Table 3a).

### Example 8

### Interleukin 18-133 C/G Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 3b
Interleukin 18 −133 C/G polymorphism allele and genotype frequencies
in the COPD patients, resistant smokers and controls.

| | 7. Allele* | | 8. Genotype | | |
|---|---|---|---|---|---|
| Frequency | G | C | GG | GC | CC |
| Controls n = 187 | 120 | 254 | 23 | 74 | 90 |
| (%) | (32%) | (68%) | (12%) | (40%) | (48%) |
| COPD n = 238 | 123 | 353² | 21 | 81 | 136¹ |
| | (26%) | (74%) | (9%) | (34%) | (57%) |
| Resistant n = 195 | 113 | 277 | 16 | 81 | 98 |
| (%) | (29%) | (71%) | (8%) | (42%) | (50%) |

*number of chromosomes (2n)

A mathematical analysis of the data in the table indicated that:
1. Genotype. CC vs CG/GG for COPD vs controls, Odds ratio (OR)=1.44, 95% confidence limits 1.0-2.2, χ² (Yates corrected)=3.4, p=0.06,
CC=susceptible to COPD
2. Allele. C vs G for COPD vs control, Odds ratio (OR)=1.36, 95% confidence limits 1.0-1.9, χ² (Yates corrected)=53.7, p=0.05
C=susceptible to COPD
   Thus, for the −133 G/C promoter polymorphism of the IL18 gene, the C allele and CC genotype were found to be significantly greater in the COPD cohort compared to the controls (OR=1.36, P=0.05 and OR=1.44, P=0.06 respectively) consistent with a susceptibility role. The CC genotype was also greater in the COPD cohort compared with resistant smokers a trend consistent with a susceptibility role (see Table 3b).

### Example 9

### Plasminogen Activator Inhibitor 1-675 4G/5G Promoter Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 4
Plasminogen activator inhibitor 1 −675 4G/5G promoter polymorphism
allele and genotype frequencies in the COPD patients, resistant smokers
and controls.

| | 9. Allele* | | 10. Genotype | | |
|---|---|---|---|---|---|
| Frequency | 5G | 4G | 5G5G | 5G4G | 4G4G |
| Controls n = 186 | 158 | 214 | 31 | 96 | 59 |
| (%) | (42%) | (58%) | (17%) | (52%) | (32%) |
| COPD n = 237 (%) | 219³ | 255 | 54^{1,2} | 111 | 72 |
| | (46%) | (54%) | (23%) | (47%) | (30%) |
| Resistant n = 194 | 152 | 236 | 31 | 90 | 73^{1,2} |
| (%) | (39%) | (61%) | (16%) | (46%) | (38%) |

*number of chromosomes (2n)

A mathematical analysis of the data in the table indicated that:
1. Genotype. 5G5G vs rest for COPD vs resistant, Odds ratio (OR)=1.55, 95% confidence limits 0.9-2.6, χ² (Yates uncorrected)=3.12, p=0.08,
5G5G=susceptible to COPD
2. Genotype. 5G5G vs rest for COPD vs control, Odds ratio (OR)=1.48, 95% confidence limits 0.9-2.5, χ² (Yates uncorrected)=2.43, p=0.12
5G5G=susceptible to COPD
3. Allele. 5G vs 4G for COPD vs resistant, Odds ratio (OR)=1.33, 95% confidence limits 1.0-1.8, χ² (Yates corrected)=4.02, p=0.05
5G=susceptible to COPD
   Thus, for the −675 4G/5G promoter polymorphism of the plasminogen activator inhibitor gene, the 5G allele and 5G5G genotype were found to be significantly greater in the COPD cohort compared to the resistant smoker cohort (OR=1.33, P=0.05 and OR=1.55, P=0.08) consistent with a susceptibility role. The greater frequency of the 5G5G in COPD compared to the blood donor cohort also suggests that the 5G5G genotype is associated with susceptibility (see Table 4).

### Example 10

### Nitric Oxide Synthase 3 Asp 298 Glu (T/G) Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 5
Nitric oxide synthase 3 Asp 298 Glu (T/G) polymorphism allele and
genotype frequencies in the COPD patients, resistant smokers
and controls.

| | 11. Allele* | | 12. Genotype | | |
|---|---|---|---|---|---|
| Frequency | T | G | TT | TG | GG |
| Controls n = 183 | 108 | 258 | 13 | 82 | 88 |
| (%) | (30%) | (70%) | (7%) | (45%) | (48%) |
| COPD n = 238 (%) | 159 | 317 | 25 | 109 | 104 |
| | (42%) | (58%) | (10%) | (47%) | (43%) |
| Resistant n = 194 | 136 | 252 | 28¹ | 80 | 86 |
| (%) | (35%) | (65%) | (15%) | (41%) | (44%) |

*number of chromosomes (2n)

A mathematical analysis of the data in the table indicated that:
1. Genotype. TT vs TG/GG for resistant vs controls, Odds ratio (OR)=2.2, 95% confidence limits 1.0-4.7, χ² (Yates corrected)=4.49, p=0.03,
TT genotype=protective for COPD
   Thus, for the 298 Asp/Glu (T/G) polymorphism of the nitric oxide synthase (NOS3) gene, the TT genotype was found to be significantly greater in the resistant smoker cohort compared to the blood donor cohort (OR=2.2, P=0.03) consistent with a protective role. (see Table 5).

### Example 11

### Vitamin D Binding Protein Lys 420 Thr (A/C) Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 6a
Vitamin D Binding Protein Lys 420 Thr (A/C) polymorphism allele and
genotype frequencies in the COPD patients, resistant smokers and
controls.

| | 13. Allele* | | 14. Genotype | | |
|---|---|---|---|---|---|
| Frequency | A | C | AA | AC | CC |
| Controls n = 189 | 113 | 265 | 17 | 79 | 93 |
| (%) | (30%) | (70%) | (9%) | (42%) | (49%) |
| COPD n = 250 (%) | 147 | 353 | 24 | 99 | 127 |
| | (29%) | (71%) | (10%) | (40%) | (50%) |
| Resistant n = 195 | 140² | 250 | 25¹ | 90¹ | 80 |
| (%) | (36%) | (64%) | (13%) | (46%) | (41%) |

*number of chromosomes (2n)

A mathematical analysis of the data in the table indicated that:
1. Genotype. AA/AC vs CC for resistant vs COPD, Odds ratio (OR)=1.39, 95% confidence limits 0.9-2.1, χ² (Yates uncorrected)=2.59, p=0.10,
AA/AC genotype=protective for COPD
2. Allele. A vs C for resistant vs COPD, Odds ratio (OR)=1.34, 95% confidence limits 1.0-1.8, χ² (Yates corrected)=3.94, p=0.05
A allele=protective for COPD
   Thus, for the Lys 420 Thr (A/C) polymorphism of the Vitamin D binding protein gene, the A allele and AA/AC genotype were found to be greater in the resistant smoker cohort compared to the COPD cohort (OR=1.34, P=0.05 and OR=1.39, P=0.10 respectively) consistent with a protective role. (see Table 6a).

### Example 12

### Vitamin D Binding Protein Glu 416 Asp (T/G) Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 6b
Vitamin D Binding Protein Glu 416 Asp (T/G) polymorphism allele and
genotype frequencies in the COPD patients, resistant smokers
and controls.

| | 15. Allele* | | 16. Genotype | | |
|---|---|---|---|---|---|
| Frequency | T | G | TT | TG | GG |
| Controls n = 188 | 162 | 214 | 35 | 92 | 61 |
| (%) | (43%) | (57%) | (19%) | (49%) | (32%) |
| COPD n = 240 (%) | 230 | 250 | 57 | 116 | 67 |
| | (48%) | (52%) | (24%) | (48%) | (28%) |
| Resistant n = 197 | 193² | 201 | 43¹ | 107¹ | 47 |
| (%) | (49%) | (51%) | (22%) | (54%) | (24%) |

*number of chromosomes (2n)

A mathematical analysis of the data in the table indicated that:
1. Genotype. TT/TG vs GG for resistant vs controls, Odds ratio (OR)=1.53, 95% confidence limits 1.0-2.5, χ² (Yates uncorrected)=3.52, p=0.06,
TT/TG genotype=protective for COPD
2. Allele. T vs G for resistant vs control, Odds ratio (OR)=1.27, 95% confidence limits 1.0-1.7, χ² (Yates corrected)=2.69, p=0.1
T allele=protective for COPD
   Thus, for the Glu 416 Asp (T/G) polymorphism of the Vitamin D binding protein gene, the T allele and TT/TG genotype were found to be greater in the resistant smoker cohort compared to the blood donor cohort cohort (OR=1.27, P=0.10 and OR=1.53, P=0.06 respectively) consistent with a protective role. (see Table 6b).

### Example 13

### Glutathione S Transferase P1 Ile 105 Val (A/G) Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 7
Glutathione S Transferase P1 Ile 105 Val (A/G) polymorphism allele and
genotype frequencies in the COPD patients, resistant smokers and
controls.

| | 17. Allele* | | 18. Genotype | | |
|---|---|---|---|---|---|
| Frequency | A | G | AA | AG | GG |
| Controls n = 185 | 232 | 138 | 70 | 92 | 23 |
| (%) | (63%) | (37%) | (38%) | (50%) | (12%) |
| COPD n = 238 (%) | 310 | 166 | 96 | 118 | 24 |
| | (65%) | (35%) | (40%) | (50%) | (10%) |
| Resistant n = 194 | 269² | 119 | 91¹ | 87 | 16 |
| (%) | (69%) | (31%) | (47%) | (45%) | (8%) |

*number of chromosomes (2n)

A mathematical analysis of the data in the table indicated that:
1. Genotype. AA vs AG/GG for resistant vs controls, Odds ratio (OR)=1.45, 95% confidence limits 0.9-2.2, χ² (Yates uncorrected)=3.19, p=0.07,
AA genotype=protective for COPD
2. Allele. A vs G for resistant vs control, Odds ratio (OR)=1.34, 95% confidence limits 1.0-1.8, χ² (Yates uncorrected)=3.71, p=0.05
A allele=protective for COPD
   Thus, for the Ile 105 Val (A/G) polymorphism of the glutathione S transferase P gene, the A allele and AA genotype were found to be greater in the resistant smoker cohort compared to the blood donor cohort (OR=1.34, P=0.05 and OR=1.45, P=0.07 respectively) consistent with a protective role. (see Table 7).

### Example 14

### Interferon-Gamma 874 A/T Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 8
Interferon-gamma 874 A/T polymorphism allele and genotype
frequencies in the COPD patients, resistant smokers
and controls.

| | 19. Allele* | | 20. Genotype | | |
|---|---|---|---|---|---|
| Frequency | A | T | AA | AT | TT |
| Controls | 183 (49%) | 189 (51%) | 37 (20%) | 109 (58%) | 40 (22%) |
| n = 186 | | | | | |
| (%) | | | | | |
| COPD | 244 (52%) | 226 (48%) | 64¹ (27%) | 116 (49%) | 55 (24%) |
| n = 235 | | | | | |
| (%) | | | | | |
| Resistant | 208 (54%) | 178 (46%) | 51 (27%) | 106 (55%) | 36 (18%) |
| n = 193 | | | | | |
| (%) | | | | | |

*number of chromosomes (2n)

A mathematical analysis of the data in the table indicated that:
1. Genotype. AA vs AT/TT for COPD vs controls, Odds ratio (OR)=1.51, 95% confidence limits 0.9-2.5, χ² (Yates uncorrected)=3.07, p=0.08,
AA genotype=susceptible to COPD
   Thus, for the 874 A/T polymorphism of the interferon-γ gene, the AA genotype was found to be significantly greater in the COPD cohort compared to the controls (OR-1.5, P=0.08) consistent with a susceptibility role. (see Table 8).

### Example 15

### Interleukin-13 Arg 130 Gln (G/A) Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 9a
Interleukin-13 Arg 130 Gln (G/A) polymorphism allele and genotype
frequencies in the COPD patients, resistant smokers and controls.

| | 21. Allele* | | 22. Genotype | | |
|---|---|---|---|---|---|
| Frequency | A | G | AA | AG | GG |
| Controls | 67 (18%) | 301 (82%) | 3 (2%) | 61 (33%) | 120 (65%) |
| n = 184 | | | | | |
| (%) | | | | | |
| COPD | 86 (18%) | 388 (82%) | 8 (3%) | 70 (30%) | 159 (67%) |
| n = 237 (%) | | | | | |
| Resistant | 74 (19%) | 314 (81%) | 9¹ (5%) | 56 (28%) | 129 (67%) |
| n = 194 | | | | | |
| (%) | | | | | |

*number of chromosomes (2n)

A mathematical analysis of the data in the table indicated that:
1. Genotype. AA vs AG/GG for resistant vs controls, Odds ratio (OR)=2.94, 95% confidence limits 0.7-14.0, χ² (Yates uncorrected)=2.78, p=0.09,
AA genotype=protective for COPD
   Thus, for the Arg 130 Gln (G/A) polymorphism of the Interleukin 13 gene, the AA genotype was found to be greater in the resistant smoker cohort compared to the blood donor cohort (OR=2.94, P=0.09) consistent with a protective role. (see Table 9a).

### Example 16

### Interleukin-13 −1055 C/T Promoter Polymorphism Allele and Genotype Frequencies in the COPD Patients, Resistant Smokers and Controls

The genotype frequency for the above allele was determined in COPD patients, resistant smokers, and controls. The frequencies are shown in the following table.

TABLE 9b
Interleukin-13-1055 C/T promoter polymorphism allele and genotype
frequencies in the COPD patients, resistant smokers and controls.

| | 23. Allele* | | 24. Genotype | | |
|---|---|---|---|---|---|
| Frequency | T | C | TT | TC | CC |
| Controls | 65 (18%) | 299 (82%) | 5 (3%) | 55 (30%) | 122 (67%) |
| n = 182 | | | | | |
| (%) | | | | | |
| COPD | 94 (20%) | 374 (80%) | 8¹ (4%) | 78 (33%) | 148 (63%) |
| n = 234 | | | | | |
| (%) | | | | | |
| Resistant | 72 (19%) | 312 (81%) | 2 (1%) | 68 (35%) | 122 (64%) |
| n = 192 | | | | | |
| (%) | | | | | |

*number of chromosomes (2n)

A mathematical analysis of the data in the table indicated that:
1. Genotype. TT vs TC/CC for COPD vs resistant, Odds ratio (OR)=6.03, 95% confidence limits 1.1-42, χ² (Yates corrected)=4.9, p=0.03,
TT=susceptible to COPD
   Thus, for the −1055 (C/T) polymorphism of the Interleukin 13 gene, the TT genotype was found to be greater in the COPD cohort compared to the resistant cohort (OR=6.03, P=0.03) consistent with a susceptibility role. (see Table 9b).

### Example 17

### α1-Antitrypsin S Polymorphism Allele and Genotype Frequencies in the COPD Patients and Resistant Smokers

The genotype frequency for the above allele was determined in COPD patients and resistant smokers. The frequencies are shown in the following table.

TABLE 10
α1-antitrypsin S polymorphism allele and genotype frequencies
in the COPD patients and resistant smokers.

| Fre- | 25. Allele* | | 26. Genotype | | |
|---|---|---|---|---|---|
| quency | M | S | MM | MS | SS |
| COPD | 391 (97%) | 13 (3%) | 189 (94%) | 13 (6%) | 0 (0%) |
| n = 202 | | | | | |
| (%) | | | | | |
| Resist- | 350 (93%) | 28 (7%) | 162 (85%) | 26¹ (14%) | 1¹ (1%) |
| ant | | | | | |
| n = 189 | | | | | |
| (%) | | | | | |

*number of chromosomes (2n)

A mathematical analysis of the data in the table indicated that:
1. Genotype. MS/SS vs MM for Resistant vs COPD, Odds ratio (OR)=2.42, 95% confidence limits 1.2-5.1, χ² (Yates corrected)=5.7, p=0.01,
S=protective for COPD
   Thus, for the α1-antitrypsin S polymorphism, the S allele and MS/SS genotype was found to be greater in the resistant smokers compared to COPD cohort (OR=2.42, P=0.01) consistent with a protective role (Table 10).

### Example 18

### Tissue Necrosis Factor α+489 G/A Polymorphism Allele and Genotype Frequency in the COPD Patients and Resistant Smokers

The genotype frequency for the above allele was determined in COPD patients and resistant smokers. The frequencies are shown in the following table.

TABLE 11a
Tissue Necrosis Factor α +489 G/A polymorphism allele and genotype
frequency in the COPD patients and resistant smokers.

| | 27. Allele* | | 28. Genotype | | |
|---|---|---|---|---|---|
| Frequency | A | G | AA | AG | GG |
| COPD | 54 (11%) | 430 (89%) | 5 (2%) | 44 (18%) | 193 (80%) |
| n = 242 (%) | | | | | |
| Resistant | 27 (7%) | 347 (93%) | 1 (1%) | 25 (13%) | 161 (86%) |
| n = 187 | | | | | |
| (%) | | | | | |

*number of chromosomes (2n)

A mathematical analysis of the data in the table indicated that:
1. Genotype. AA/AG vs GG for COPD vs resistant, Odds ratio (OR)=1.57, 95% confidence limits 0.9-2.7, χ² (Yates corrected)=2.52, p=0.11,
AA/AG=susceptible (GG=protective)
2. Allele. A vs G for COPD vs resistant, Odds ratio (OR)=1.61, 95% confidence limits 1.0-2.7, χ² (Yates corrected)=3.38, p=0.07,
A=susceptible
   Thus, for the +489 G/A polymorphism of the Tissue Necrosis Factor α gene, the A allele and the AA and AG genotypes were found to be greater in the COPD cohort compared to the controls (OR=1.57, P=0.11) consistent with a susceptibility role (see Table 11a). Conversely, the GG genotype was found to be greater in the resistant smoker cohort, consistent with a protective role (see Table 11a).

### Example 19

### Tissue Necrosis Factor α −308 G/A Polymorphism Allele and Genotype Frequency in the COPD Patients and Resistant Smokers

The genotype frequency for the above allele was determined in COPD patients and resistant smokers. The frequencies are shown in the following table.

TABLE 11b
Tissue Necrosis Factor α −308 G/A polymorphism allele and genotype
frequency in the COPD patients and resistant smokers.

| | 29. Allele* | | 30. Genotype | | |
|---|---|---|---|---|---|
| Frequency | A | G | AA | AG | GG |
| COPD | 90 (19%) | 394 (81%) | 6 (2%) | 78 (32%) | 158 (65%) |
| n = 242 | | | | | |
| (%) | | | | | |
| Resistant | 58 (15%) | 322 (85%) | 3 (2%) | 52 (27%) | 135 (71%) |
| n = 190 | | | | | |
| (%) | | | | | |

*number of chromosomes (2n)

A mathematical analysis of the data in the table indicated that:
1. Genotype. GG vs AG/AA for COPD vs resistant, Odds ratio (OR)=0.77, 95% confidence limits 0.5-1.2, χ² (Yates uncorrected)=1.62, p=0.20,
GG=protective (AA/AG susceptible) trend
2. Allele. A vs G for COPD vs resistant, Odds ratio (OR)=1.3, 95% confidence limits 0.9-1.9, χ² (Yates uncorrected)=1.7, p=0.20,
A=susceptible trend
   Thus, for the −308 G/A polymorphism of the Tissue Necrosis Factor α gene, the GG genotype was found to be greater in the resistant smoker cohort compared to the COPD cohort (OR=0.77, P=0.20) consistent with a protective role. (see Table 11b). Conversely, the A allele and the AA and AG genotypes were found to be greater in the COPD cohort (OR=1.3, P=0.20), consistent with a susceptibility role (see Table 11b).

### Example 20

### SMAD3 C89Y Polymorphism Allele and Genotype Frequency in the COPD Patients and Resistant Smokers

The genotype frequency for the above allele was determined in COPD patients and resistant smokers. The frequencies are shown in the following table.

TABLE 12
SMAD3 C89Y polymorphism allele and genotype frequency
in the COPD patients and resistant smokers.

| | 31. Allele* | | 32. Genotype | | |
|---|---|---|---|---|---|
| Frequency | A | G | AA | AG | GG |
| COPD n = 250 (%) | 2 (1%) | 498 (99%) | 0 (0%) | 2 (1%) | 248 (99%) |
| Resistant n = 196 | 6 (2%) | 386 (98%) | 0 (0%) | 6 (3%) | 190 (97%) |
| (%) | | | | | |

*number of chromosomes (2n)

A mathematical analysis of the data in the table indicated that:
1. Genotype. AA/AG vs GG for COPD vs resistant, Odds ratio (OR)=0.26, 95% confidence limits 0.04-1.4, χ² (Yates uncorrected)=3.19, p=0.07,
AA/AG=protective (GG susceptible)
   Thus, for the C89Y A/G polymorphism of the SMAD3 gene, the AA and AG genotypes were found to be greater in the resistant smoker cohort compared to the COPD cohort (OR=0.26, P=0.07) consistent with a protective role. (see Table 12). Conversely, the GG genotype was found to be greater in the COPD cohort, consistent with a susceptibility role (see Table 12).

### Example 21

### Intracellular Adhesion Molecule 1 (ICAM1) A/G E469K (rs5498) Polymorphism Allele and Genotype Frequency in COPD Patients and Resistant Smokers

The genotype frequency for the above allele was determined in COPD patients and resistant smokers. The frequencies are shown in the following table.

TABLE 13
Intracellular Adhesion molecule 1 (ICAM1) A/G E469K (rs5498)
polymorphism allele and genotype frequency in COPD patients
and resistant smokers.

| | 33. Allele* | | 34. Genotype | | |
|---|---|---|---|---|---|
| Frequency | A | G | AA | AG | GG |
| COPD | 259 (54%) | 225 (46%) | 73 (30%) | 113 (47%) | 56 (23%) |
| n = 242 | | | | | |
| (%) | | | | | |
| Resistant | 217 (60%) | 147 (40%) | 64 (35%) | 89 (49%) | 29 (16%) |
| n = 182 | | | | | |
| (%) | | | | | |

*number of chromosomes (2n)

A mathematical analysis of the data in the table indicated that:
1. Genotype. GG vs AG/GG for COPD vs resistant, Odds ratio (OR)=1.60, 95% confidence limits 0.9-2.7, χ² (Yates corrected)=3.37, p=0.07,
GG=susceptibility
2. Allele. G vs A for COPD vs resistant, Odds ratio (OR)=1.3, 95% confidence limits 1.0-1.7, χ² (Yates corrected)=2.90, p=0.09
   Thus, for the E469K A/G polymorphism of the Intracellular adhesion molecule 1 gene, the G allele and the GG genotype were found to be greater in the COPD cohort compared to the controls (OR=1.3, P=0.09 and OR=1.6, P=0.07, respectively) consistent with a susceptibility role (see Table 13).

### Example 22

### Caspase (NOD2) Gly881Arg Polymorphism Allele and Genotype Frequencies in the COPD Patients and Resistant Smokers

The genotype frequency for the above allele was determined in COPD patients and resistant smokers. The frequencies are shown in the following table.

TABLE 14
Caspase (NOD2) Gly881Arg polymorphism allele and genotype
frequencies in the COPD patients and resistant smokers.

| | 35. Allele* | | 36. Genotype | | |
|---|---|---|---|---|---|
| Frequency | G | C | GG | GC | CC |
| COPD | 486 (98%) | 8 (2%) | 239 (97%) | 8 (3%) | 0 (0%) |
| n = 247 | | | | | |
| Resistant | 388 (99.5%) | 2 0.5%) | 193 (99%) | 2 (1%) | 0 (0%) |
| n = 195 | | | | | |
| (%) | | | | | |

*number of chromosomes (2n)

A mathematical analysis of the data in the table indicated that:
1. Genotype. CC/CG vs GG for COPD vs resistant, Odds ratio (OR)=3.2, 95% confidence limits 0.6-22, χ² (Yates uncorrected)=2.41, p=0.11 (1-tailed),
GC/CC=susceptibility (trend)
   Thus, for the Gly 881Arg G/C polymorphism of the Caspase (NOD2) gene, the CC and CG genotypes were found to be greater in the COPD cohort compared to the controls (OR=3.2, P=0.11) consistent with a susceptibility role (see Table 14).

### Example 23

### Mannose Binding Lectin 2(MBL2) +161 G/A Polymorphism Allele and Genotype Frequencies in the COPD Patients and Resistant Smokers

The genotype frequency for the above allele was determined in COPD patients and resistant smokers. The frequencies are shown in the following table.

TABLE 15
Mannose binding lectin 2(MBL2) +161 G/A polymorphism allele
and genotype frequencies in the COPD patients and
resistant smokers.

| | 37. Allele* | | 38. Genotype | | |
|---|---|---|---|---|---|
| Frequency | A | G | AA | AG | GG |
| COPD | 110 (25%) | 326 (75%) | 6 (3%) | 98 (45%) | 114 (52%) |
| n = 218 (%) | | | | | |
| Resistant | 66 (18%) | 300 (82%) | 6 (3%) | 54 (30%) | 123 (67%) |
| n = 183 | | | | | |
| (%) | | | | | |

*number of chromosomes (2n)

A mathematical analysis of the data in the table indicated that:
1. Genotype. GG vs rest for COPD vs resistant, Odds ratio (OR)=0.53, 95% confidence limits 0.4-0.80, χ² (Yates uncorrected)=8.55, p=0.003,
GG=protective
   Thus, for the 161 G/A polymorphism of the Mannose binding lectin 2 gene, the GG genotype was found to be greater in the resistant smoker cohort compared to the COPD cohort (OR=0.53, P=0.003) consistent with a protective role. (see Table 15).

### Example 24

### Chymase 1 (CMA1)-1903 G/A Promoter Polymorphism Allele and Genotype Frequencies in the COPD Patients and Resistant Smokers

The genotype frequency for the above allele was determined in COPD patients and resistant smokers. The frequencies are shown in the following table.

TABLE 16
Chymase 1 (CMA1) −1903 G/A promoter polymorphism allele
and genotype frequencies in the COPD patients and resistant smokers.

| | Frequency | | | | |
|---|---|---|---|---|---|
| | 39. Allele* | | 40. Genotype | | |
| | A | G | AA | AG | GG |
| COPD n = 239 | 259 | 219 | 67 (28%) | 125 (52%) | 47 (20%) |
| (%) | (54%) | (46%) | | | |
| Resistant n = 181 | 209 | 153 | 63 (35%) | 83 (46%) | 35 (19%) |
| (%) | (58%) | (42%) | | | |

*number of chromosomes (2n)

A mathematical analysis of the data in the table indicated that:
1. Genotype. AA vs AG/GG for COPD vs resistant, Odds ratio (OR)=0.73, 95% confidence limits 0.5-1.1, χ² (Yates corrected)=1.91, p=0.17,
AA genotype=protective trend
   Thus, for the −1903 G/A polymorphism of the Chymase 1 gene, the AA genotype was found to be greater in the resistant smoker cohort compared to the COPD cohort (OR=0.73, P=0.17) consistent with a protective role. (see Table 16).

### Example 25

### N-Acetyltransferase 2 Arg 197 Gln G/A Polymorphism Allele and Genotype Frequencies in COPD and Resistant Smokers

The genotype frequency for the above allele was determined in COPD patients and resistant smokers. The frequencies are shown in the following table.

TABLE 17
N-Acetyltransferase 2 Arg 197 Gln G/A polymorphism allele
and genotype frequencies in COPD and resistant smokers.

| | Frequency | | | | |
|---|---|---|---|---|---|
| | 41. Allele* | | 42. Genotype | | |
| | A | G | AA | AG | GG |
| COPD n = 247 | 136 | 358 | 14 (6%) | 108 (44%) | 125 (50%) |
| (%) | (28%) | (72%) | | | |
| Resistant n = 196 | 125 | 267 | 21 (11%) | 83 (42%) | 92 (47%) |
| (%) | (32%) | (68%) | | | |

*number of chromosomes (2n)

A mathematical analysis of the data in the table indicated that:
1. Genotype. AA vs AG/GG for COPD vs resistant, Odds ratio (OR)=0.50, 95% confidence limits 0.2-1.0, χ² (Yates uncorrected)=3.82, p=0.05,
AA genotype=protective
   Thus, for the Arg 197 Gln G/A polymorphism of the N-Acetyl transferase 2 gene, the AA genotype was found to be greater in the resistant smoker cohort compared to the COPD cohort (OR=0.50, P=0.05) consistent with a protective role. (see Table 17).

### Example 26

### Interleukin 1B (IL-1b) −511 A/G Polymorphism Allele and Genotype Frequencies in COPD and Resistant Smokers

The genotype frequency for the above allele was determined in COPD patients and resistant smokers. The frequencies are shown in the following table.

TABLE 18
Interleukin 1B (IL-1b) −511 A/G polymorphism allele
and genotype frequencies in COPD and resistant smokers.

| | Frequency | | | | |
|---|---|---|---|---|---|
| | 43. Allele* | | 44. Genotype | | |
| | A | G | AA | AG | GG |
| COPD n = 248 | 160 | 336 | 31 (13%) | 98 (40%) | 119 (48%) |
| (%) | (32%) | (68%) | | | |
| Resistant n = 195 | 142 | 248 | 27 (14%) | 88 (45%) | 80 (41%) |
| (%) | (36%) | (64%) | | | |

*number of chromosomes (2n)

A mathematical analysis of the data in the table indicated that:
1. Genotype. GG vs AA/AG for COPD vs resistant, Odds ratio (OR)=1.3, 95% confidence limits 0.9-2.0, χ² (Yates corrected)=1.86, p=0.17,
GG genotype=susceptible trend
   Thus, for the −511 A/G polymorphism of the Interleukin 1B gene, the GG genotype was found to be greater in the COPD cohort compared to the controls (OR=1.3, P=0.17) consistent with a susceptibility role (see Table 18).

### Example 27

### Microsomal Epoxide Hydrolase (MEH) Tyr 113 His T/C (Exon 3) Polymorphism Allele and Genotype Frequency in COPD and Resistant Smokers

The genotype frequency for the above allele was determined in COPD patients and resistant smokers. The frequencies are shown in the following table.

TABLE 19a
Microsomal epoxide hydrolase (MEH) Tyr 113 His T/C
(exon 3) polymorphism allele and genotype frequency
in COPD and resistant smokers.

| | Frequency | | | | |
|---|---|---|---|---|---|
| | 45. Allele* | | 46. Genotype | | |
| | C | T | CC | CT | TT |
| COPD n = 249 | 137 | 361 | 18 (7%) | 101 (41%) | 130 (52%) |
| (%) | (28%) | (72%) | | | |
| Resistant n = 194 | 130 | 258 | 19 (10%) | 92 (47%) | 83 (43%) |
| (%) | (34%) | (66%) | | | |

*number of chromosomes (2n)

A mathematical analysis of the data in the table indicated that:
1. Genotype. TT vs CT/CC for COPD vs resistant, Odds ratio (OR)=1.5, 95% confidence limits 1.0-2.2, χ² (Yates corrected)=3.51, p=0.06,
TT genotype=susceptible
   Thus, for the Tyr 113 His T/C polymorphism of the Microsomal epoxide hydrolase gene, the TT genotype was found to be greater in the COPD cohort compared to the controls (OR=1.5, P=0.06) consistent with a susceptibility role (see Table 19a).

### Example 28

### Microsomal Epoxide Hydrolase (MEH) His 139 Arg A/G (Exon 4) Polymorphism Allele and Genotype Frequency in COPD and Resistant Smokers

The genotype frequency for the above allele was determined in COPD patients and resistant smokers. The frequencies are shown in the following table.

TABLE 19b
Microsomal epoxide hydrolase (MEH) His 139 Arg A/G
(exon 4) polymorphism allele and genotype frequency
in COPD and resistant smokers.

| | Frequency | | | | |
|---|---|---|---|---|---|
| | 47. Allele* | | 48. Genotype | | |
| | A | G | AA | AG | GG |
| COPD n = 238 (%) | 372 | 104 (22%) | 148 | 76 (32%) | 14 (6%) |
| | (78%) | | (62%) | | |
| Resistant n = 179 | 277 | 81 (23%) | 114 | 49 (27%) | 16 (9%) |
| (%) | (77%) | | (64%) | | |

*number of chromosomes (2n)

1. Genotype. GG vs AA/AG for COPD vs resistant, Odds ratio (OR)=0.64, 95% confidence limits 0.3-1.4, χ² (Yates uncorrected)=1.43, p=0.23,
GG genotype=protective (trend)
   Thus, for the Arg 139 G/A polymorphism of the Microsomal epoxide hydrolase gene, the GG genotype was found to be greater in the resistant smoker cohort compared to the COPD cohort (OR=0.64, P=0.23) consistent with a protective role. (see Table 19b).

### Example 29

### Lipo-Oxygenase −366 G/A Polymorphism Allele and Genotype Frequencies in the COPD Patient and Resistant Smokers

The genotype frequency for the above allele was determined in COPD patients and resistant smokers. The frequencies are shown in the following table.

TABLE 20
Lipo-oxygenase −366 G/A polymorphism allele and genotype
frequencies in the COPD patients and resistant smokers.

| | Frequency | | | | |
|---|---|---|---|---|---|
| | 49. Allele* | | 50. Genotype | | |
| | A | G | AA | AG | GG |
| COPD n = 247 | 21 (4%) | 473 | 1 (0.5%) | 19 (7.5%) | 227 (92%) |
| (%) | | (96%) | | | |
| Resistant n = 192 | 25 (7%) | 359 | 0 (0%) | 25 (13%) | 167 (87%) |
| (%) | | (93%) | | | |

*number of chromosomes (2n)

1. Genotype. AA/AG vs GG for COPD vs resistant, Odds ratio (OR)=0.60, 95% confidence limits 0.3-1.1, χ² (Yates corrected)=2.34, p=0.12,
AA/AG genotype=protective (GG susceptible) trend
   Thus, for the −366 G/A polymorphism of the 5 Lipo-oxygenase gene, the AG and AA genotypes were found to be greater in the resistant smoker cohort compared to the COPD cohort (OR=0.60, P=0.12) consistent with a protective role. (see Table 20). Conversely, the GG genotype was found to be greater in the COPD cohort, consistent with a susceptibility role (see Table 20).

### Example 30

### Heat Shock Protein 70 (HSP 70) HOM T2437C Polymorphism Allele and Genotype Frequencies in the COPD Patients and Resistant Smokers

The genotype frequency for the above allele was determined in COPD patients and resistant smokers. The frequencies are shown in the following table.

TABLE 21
Heat Shock Protein 70 (HSP 70) HOM T2437C polymorphism allele
and genotype frequencies in the COPD patients and resistant smokers.

| | Frequency | | | | |
|---|---|---|---|---|---|
| | 51. Allele* | | 52. Genotype | | |
| | C | T | CC | CT | TT |
| COPD n = 199 | 127 (32%) | 271 | 5 (3%) | 117 (59%) | 77 (39%) |
| (%) | | (68%) | | | |
| Resistant n = 166 | 78 (23%) | 254 | 4 (2%) | 70 (42%) | 92 (56%) |
| (%) | | (77%) | | | |

*number of chromosomes (2n)

1. Genotype. CC/CT vs TT for COPD vs resistant, Odds ratio (OR)=2.0, 95% confidence limits 1.3-3.1, χ² (Yates uncorrected)=9.52, p=0.002,
CC/CT genotype=susceptible (TT=protective)
   Thus, for the HOM T2437C polymorphism of the Heat Shock Protein 70 gene, the CC and CT genotypes were found to be greater in the COPD cohort compared to the controls (OR=2.0, P=0.002) consistent with a susceptibility role (see Table 21). Conversely, the TT genotype was found to be greater in the resistant smoker cohort, consistent with a protective role (see Table 21).

### Example 31

### Chloride Channel Calcium-Activated 1 (CLCA1) +13924 T/A Polymorphism Allele and Genotype Frequencies in the COPD Patients and Resistant Smokers

The genotype frequency for the above allele was determined in COPD patients and resistant smokers. The frequencies are shown in the following table.

TABLE 22
Chloride Channel Calcium-activated 1 (CLCA1) +13924 T/A
polymorphism allele and genotype frequencies in the COPD patients
and resistant smokers.

| | Frequency | | | | |
|---|---|---|---|---|---|
| | 53. Allele* | | 54. Genotype | | |
| | A | T | AA | AT | TT |
| COPD n = 224 | 282 | 166 | 84 (38%) | 114 (51%) | 26 (12%) |
| (%) | (63%) | (37%) | | | |
| Resistant n = 158 | 178 | 138 | 42 (27%) | 94 (59%) | 22 (14%) |
| (%) | (56%) | (44%) | | | |

*number of chromosomes (2n)

1. Genotype. AA vs AT/TT for COPD vs resistant, Odds ratio (OR)=1.7, 95% confidence limits 1.0-2.7, χ² (Yates corrected)=4.51, p=0.03,
AA=susceptible
   Thus, for the +13924 T/A polymorphism of the Chloride Channel Calcium-activated 1 gene, the AA genotype was found to be greater in the COPD cohort compared to the controls (OR=1.7, P=0.03) consistent with a susceptibility role (see Table 22).

### Example 32

### Monocyte Differentiation Antigen CD-14 −159 Promoter Polymorphism Allele and Genotype Frequencies in the COPD Patients and Resistant Smokers

The genotype frequency for the above allele was determined in COPD patients and resistant smokers. The frequencies are shown in the following table.

TABLE 23
Monocyte differentiation antigen CD-14 −159 promoter
polymorphism allele and genotype frequencies in the
COPD patients and resistant smokers.

| | Frequency | | | | |
|---|---|---|---|---|---|
| | 55. Allele* | | 56. Genotype | | |
| | C | T | CC | CT | TT |
| COPD n = 240 | 268 | 212 | 77 (32%) | 114 (48%) | 49 (20%) |
| (%) | (56%) | (44%) | | | |
| Resistant n = 180 | 182 | 178 | 46 (25%) | 90 (50%) | 44 (24%) |
| (%) | (51%) | (49%) | | | |

*number of chromosomes (2n)

1. Genotype.CC vs CT/TT for COPD vs Resistant, Odds ratio (OR)=1.4, 95% confidence limits 0.9-2.2, χ² (Yates uncorrected)=2.12, p=0.15,
CC=susceptible (trend)
   Thus, for the −159 C/T polymorphism of the Monocyte differentiation antigen CD-14 gene, the CC genotype was found to be greater in the COPD cohort compared to the controls (OR=1.4, P=0.15) consistent with a susceptibility role (see Table 23).

### Example 33

### Elafin +49 C/T Polymorphism Allele and Genotype Frequencies in the COPD Patients, Resistant Smokers and Controls

The genotype frequency for the above allele was determined in COPD patients and resistant smokers. The frequencies are shown in the following table.

TABLE 24
Elafin +49 C/T polymorphism allele and genotype frequencies
in the COPD patients, resistant smokers and controls.

| | Frequency | | | | |
|---|---|---|---|---|---|
| | 57. Allele* | | 58. Genotype | | |
| | C | T | CC | CT | TT |
| COPD n = 144 (%) | 247 | 41 | 105 (73%) | 37 (26%) | 2 (1%) |
| | (86%) | (14%) | | | |
| Resistant n = 75 | 121 | 29 | 49 (65%) | 23 (31%) | 3 (4%) |
| (%) | (81%) | (19%) | | | |

*number of chromosomes (2n)

1. Genotype. CT/TT vs CC for COPD vs resistant, Odds ratio (OR)=0.70, 95% confidence limits=0.4-1.3, χ² (Yates uncorrected)=1.36, p=0.24,
CT/TT genotype=protective (trend only)
2. Allele: T vs C for COPD vs resistant, Odds ratio (OR)=0.69, 95% confidence limits=0.4-1.2, χ² (Yates uncorrected)=1.91, p=0.17,
T genotype=protective (trend only)
   Thus, for the Exon 1 +49 C/T polymorphism of the Elafin gene, the T allele and the CT and TT genotypes were found to be greater in the resistant smoker cohort compared to the COPD cohort (OR=0.69, P=0.17, OR=0.70, P=0.24, respectively) consistent with a protective role. (see Table 24).

### Example 34

### Beta2-Adrenoreceptor Gln 27 Glu Polymorphism Allele and Genotype Frequency in the COPD Patients, Resistant Smokers and Controls

The genotype frequency for the above allele was then determined in COPD patients, resistant smokers, and controls. The frequencies are shown in the following table.

TABLE 25
Beta2-adrenoreceptor Gln 27 Glu polymorphism allele and genotype
frequency in the COPD patients, resistant smokers and controls.

| | |
|---|---|
| 59. Allele* | 60. Genotype |

| Frequency | C | G | CC | CG | GG |
|---|---|---|---|---|---|
| Controls | 204 (55%) | 168 (45%) | 57 (31%) | 89 (48%) | 39 (21%) |
| n = 185 | | | | | |
| (%) | | | | | |
| COPD | 268 (56%) | 208 (44%) | 67 (28%) | 134 (56%) | 37 (16%) |
| n = 238 (%) | | | | | |
| Resistant | 220 (56%) | 170 (44%) | 64 (33%) | 92 (47%) | 39 (20%) |
| n = 195 | | | | | |
| (%) | | | | | |

*number of chromosomes (2n)

1. Genotype. GG vs CG/CC for COPD vs resistant, Odds ratio (OR)=0.74, 95% confidence limits=0.4-1.2, χ² (Yates uncorrected)=1.47, p=0.23,
GG=protective (trend)
2. Genotype. GG vs CG/CC for COPD vs controls, Odds ratio (OR)=0.69, 95% confidence limits=0.4-1.2, χ² (Yates uncorrected)=2.16, p=0.14,
GG=protective (trend)
   Thus, for the Gln 27 Glu C/G polymorphism of the β2-adrenergic receptor gene, the GG genotype was found to be greater in the resistant smoker cohort and the blood donor controls compared to the COPD cohort (OR=0.74, P=0.23, OR=0.69, P=0.14, respectively) consistent with a protective role. (see Table 25).

### Example 35

### Maxtrix Metalloproteinase 1 (MMP1) −1607 1G/2G Polymorphism Allele and Genotype Frequencies in COPD Patients, Resistant Smokers and Controls

The genotype frequency for the above allele was then determined in COPD patients, resistant smokers, and controls. The frequencies are shown in the following table.

TABLE 26
Maxtrix metalloproteinase 1 (MMP1) −1607 1G/2G polymorphism
allele and genotype frequencies in COPD patients, resistant smokers
and controls.

| | 61. Allele* | | 62. Genotype | | |
|---|---|---|---|---|---|
| Frequency | 1G | 2G | 1G1G | 1G2G | 2G2G |
| Controls | 214 (61%) | 134 (39%) | 68 (39%) | 78 (45%) | 28 (16%) |
| n = 174 | | | | | |
| (%) | | | | | |
| COPD | 182 (42%) | 252 (58%) | 47 (22%) | 88 (41%) | 82 (38%) |
| n = 217 (%) | | | | | |
| Resistant | 186 (50%) | 188 (50%) | 46 (25%) | 94 (50%) | 47 (25%) |
| n = 187 | | | | | |
| (%) | | | | | |

*number of chromosomes (2n)

1. Genotype. 1G6G vs rest for COPD vs controls, Odds ratio (OR)=0.43, 95% confidence limits 0.3-0.7, χ² (Yates uncorrected)=13.3, p=0.0003
1G6G genotype=protective
2. Allele. 1G vs 2G for COPD vs controls, Odds ration (OR)=0.45, 95% confidence limits 0.3-0.6, χ² (Yates corrected)=28.8, p<0.0001,
1G=protective
3. Genotype. 1G1G/1G2G vs rest for COPD vs resistant smokers, Odds ratio (OR)=0.55, 95% confidence limits 0.4-0.9, χ² (Yates uncorrected)=6.83, p=0.009
1G1G/162G genotypes=protective
4. Allele. 1G vs 2G for COPD vs resistant smokers, Odds ratio (OR)=0.73, 95% confidence limits 0.6-1.0, χ² (Yates corrected)=4.61, p=0.03,
1G=protective
5. Genotype. 2G2G vs 1G1G/1G2G for COPD vs controls, Odds ratio (OR)=3.17, 95% confidence limits 1.9-5.3, χ² (Yates uncorrected)=21.4, p<0.0001
2G2G genotype=susceptible
6. Allele. 2G vs 1G for COPD vs controls, Odds ratio (OR)=2.2, 95% confidence limits 1.6-3.0, χ² (Yates corrected)=28.8, p<0.00001,
2G=susceptible
7. Genotype. 2G2G vs 1G1G/1G2G for COPD vs resistant, Odds ratio (OR)=1.81, 95% confidence limits 1.2-2.9, χ² (Yates uncorrected)=6.83, p=0.009
2G2G genotype=susceptible
8. Allele. 2G vs 1G for COPD vs resistant, Odds ratio (OR)=1.4, 95% confidence limits 1.0-1.8, χ² (Yates corrected)=4.61, p=0.0.03,
2G=susceptible
   Thus, for the −1607 1G/2G promoter polymorphism of the MMP1 gene, the 1G allele and 1G1G/1G2G genotypes were found to be significantly greater in the resistant smoker cohort compared to the COPD cohort (OR=0.73, p=0.03 and OR=0.55, p=0.009), consistent with a protective role. The greater frequency of the 1G1G in the resistant group compared to the blood donor cohort also suggests that the 1G allele is protective (see Table 26).

Table 27 summarizes the above results and examples.

TABLE 27
Protective and susceptibility polymorphisms

| Gene | Polymorphism | Role |
|---|---|---|
| Cyclo-oxygenase 2 (COX2) | COX2 −765 G/C | CC/CG protective |
| β2-adrenoreceptor (ADBR) | ADBR Arg 16 Gly | GG susceptible |
| Interleukin-18 (IL18) | IL18 −133 C/G | CC susceptible |
| Interleukin-18 (IL18) | IL18 105 A/C | AA susceptible |
| Plasminogen activator inhibitor 1 (PAI-1) | PAI-1 −675 4G/5G | 5G5G susceptible |
| Nitric Oxide synthase 3 (NOS3) | NOS3 298 Asp/Glu | TT protective |
| Vitamin D Binding Protein (VDBP) | VDBP Lys 420 Thr | AA/AC protective |
| Vitamin D Binding Protein (VDBP) | VDBP Glu 416 Asp | TT/TG protective |
| Glutathione S Transferase (GSTP-1) | GSTP1 Ile 105 Val | AA protective |
| Interferon γ (IFN-γ) | IFN-γ 874 A/T | AA susceptible |
| Interleukin-13 (IL13) | IL13 Arg 130 Gln | AA protective |
| Interleukin-13 (IL13) | Il13 −1055 C/T | TT susceptible |
| α1-antitrypsin (α1-AT) | α1-AT S allele | MS protective |
| Tissue Necrosis Factor α TNFα | TNFα +489 G/A | AA/AG susceptible |
| | | GG protective |
| Tissue Necrosis Factor α TNFα | TNFα −308 G/A | GG protective |
| | | AA/AG susceptible |
| SMAD3 | SMAD3 C89Y AG | AA/AG protective |
| | | GG susceptible |
| Intracellular adhesion molecule 1 (ICAM1) | ICAM1 E469K A/G | GG susceptible |
| Caspase (NOD2) | NOD2 Gly 881 Arg G/C | GC/CC susceptible |
| Mannose binding lectin 2 (MBL2) | MBL2 161 G/A | GG protective |
| Chymase 1 (CMA1) | CMA1 −1903 G/A | AA protective |
| N-Acetyl transferase 2 (NAT2) | NAT2 Arg 197 Gln | AA protective |
| | G/A | |
| Interleukin 1B (IL1B) | (IL1B) −511 A/G | GG susceptible |
| Microsomal epoxide hydrolase (MEH) | MEH Tyr 113 His T/C | TT susceptible |
| Microsomal epoxide hydrolase (MEH) | MEH His 139 Arg G/A | GG protective |
| 5 Lipo-oxygenase (ALOX5) | ALOX5 −366 G/A | AA/AG protective |
| | | GG susceptible |
| Heat Shock Protein 70 (HSP 70) | HSP 70 HOM T2437C | CC/CT susceptible |
| | | TT protective |
| Chloride Channel Calcium-activated 1 (CLCA1) | CLCA1 +13924 T/A | AA susceptible |
| Monocyte differentiation antigen CD-14 | CD-14 −159 C/T | CC susceptible |
| Elafin | Elafin Exon 1 +49 C/T | CT/TT protective |
| B2-adrenergic receptor (ADBR) | ADBR Gln 27 Glu C/G | GG protective |
| Matrix metalloproteinase 1 (MMP1) | MMP1 −1607 1G/2G | 1G1G/1G2G |
| | | protective |

### Example 36

In addition to examining the individual frequencies, the frequencies of the presence or absence of protective genotypes in various combinations were also examined. The results are summarized in Table 28.

TABLE 28
Combined frequencies of the presence or absence of selected protective
genotypes (COX2 (−765) CC/CG, β2 adreno-receptor AA, Interleukin-13 AA,
Nitic Oxide Synthase 3 TT and Vitamin D Binding Protein AA) in
the smoking subjects (COPD subjects and resistant smokers).

| Cohorts | 0 | | 1 | | ≧2 | | Total |
|---|---|---|---|---|---|---|---|
| COPD | 136 | (54%) | 100 | (40%) | 16 | (7%) | 252 |
| Resistant smokers | 79 | (40%) | 83 | (42%) | 34 | (17%) | 196 |
| % of smokers with COPD | 136/215 | (63%) | 100/183 | (55%) | 16/50 | (32%) | |

| Comparison | Odd's ratio | 95% CI | χ² | P value |
|---|---|---|---|---|
| 0 vs 1 vs 2+, Resist vs COPD | — | — | 16.43 | 0.0003 |
| 2+ vs 0-1, Resist vs COPD | 3.1 | 1.6-6.1 | 12.36 | 0.0004 |
| 1+ vs 0, Resist vs COPD | 1.74 | 1.2-2.6 | 7.71 | 0.006 |

### Example 37

In addition to examining the frequencies of particular susceptibility genotypes individually, the combined frequencies of multiple susceptibility genotypes was also examined. In particular, this example examines the combined frequencies of the presence or absence of selected susceptibility genotypes (Interleukin-18 105 AA, PAI-1-675 5G5G, Interleukin-13—1055 TT and Interferon-γ −874 TT genotypes) in the smoking subjects (COPD subjects and resistant smokers). The results are summarized in Table 29.

TABLE 29

| | Number of susceptibility polymorphisms | | | | | | |
|---|---|---|---|---|---|---|---|
| Cohorts | 0 | | 1 | | ≧2 | | Total |
| COPD | 66 | (26%) | 113 | (45%) | 73 | (29%) | 252 |
| Resistant smokers | 69 | (35%) | 92 | (47%) | 35 | (18%) | 196 |
| % of smokers with COPD | 66/135 | (49%) | 113/205 | (55%) | 73/108 | (68%) | |

| Comparison | Odd's ratio | 95% CI | χ² | P value |
|---|---|---|---|---|
| 0 vs 1 vs 2+, COPD vs Resist | — | — | 8.72 | 0.01 |
| 2+ vs 0-1, COPD vs Resist | 1.9 | 1.2-3.0 | 6.84 | 0.009 |
| 1+ vs 0, COPD vs Resist | 1.5 | 1.0-3.5 | 3.84 | 0.05 |

### Example 38

In addition to examining the individual frequencies, the combined frequencies of the presence or absence of protective genotypes was also examined. In particular, this example examined the combined frequencies of the presence or absence of COX2 (−765) CC/CG, Interleukin-13 AA, Nitic Oxide Synthase 3 TT, Vitamin D Binding Protein AA/AC, GSTP1 AA and α1-antitrypsin MS/SS in the smoking subjects (COPD subjects and resistant smokers). The results are summarized in Table 30.

TABLE 30

| | Number of protective polymorphisms | | | | | | |
|---|---|---|---|---|---|---|---|
| Cohorts | 0 | | 1 | | ≧2 | | Total |
| COPD | 51 | (19%) | 64 | (24%) | 150 | (57%) | 265 |
| Resistant smokers | 16 | (8%) | 56 | (27%) | 133 | (65%) | 205 |
| % of smokers with COPD | 51/76 | (76%) | 64/120 | (53%) | 150/283 | (53%) | |

| Comparison | Odd's ratio | 95% CI | χ² | P value |
|---|---|---|---|---|
| 0 vs 1 vs 2+, Resist vs COPD | — | — | 12.14 | 0.0005 |
| 1+ vs 0, Resist vs COPD | 2.82 | 1.5-5.3 | 11.46 | 0.0004 |

The above Examples demonstrate that several polymorphisms were associated with either susceptibility and/or resistance to obstructive lung disease in those exposed to smoking environments. Additionally, while the associations of individual polymorphisms on their own, did provide discriminatory value, did not necessarily offer the most accurate prediction of disease. However, in combination these polymorphisms distinguish susceptible smokers (with COPD) from those who are resistant. The polymorphisms represent both promoter polymorphisms, thought to modify gene expression and hence protein synthesis, and exonic polymorphisms known to alter amino-acid sequence (and likely expression and/or function) in processes known to underlie lung remodelling. The polymorphisms identified here are found in genes encoding proteins central to these processes which include inflammation, matrix remodelling and oxidant stress.

In the comparison of smokers with COPD and matched smokers with near normal lung function, several polymorphisms were identified as being found in significantly greater or lesser frequency than in the comparator groups (including the blood donor cohort).
In the analysis of the −765 C/G promoter polymorphisms of cyclo-oxygenase 2 gene, the C allele and CC/CG genotype were found to be significantly greater in the resistant smoker cohort compared to the COPD cohort (OR=1.92, P<0.001 and OR=1.98, P=0.003) consistent with a protective role. The greater frequency compared to the blood donor cohort also suggests that the C allele (CC genotype) is over represented in the resistant group (see Table 1C).
In the analysis of the Arg16Gly polymorphism of the β2 adrenergic receptor gene, the GG genotype was found to be significantly greater in the COPD cohort compared to the controls (OR=1.83, P=0.004) suggesting a possible susceptibility to smoking associated with this genotype. Although the GG genotype is also over-represented in the resistant cohort its effects can be overshadowed by protective polymorphisms (see Table 2).
In the analysis of the 105 C/A polymorphism of the IL18 gene, the A allele and AA genotype were found to be significantly greater in the COPD cohort compared to the controls (OR=1.46, P=0.02 and OR=1.50, P=0.04 respectively) consistent with a susceptibility role. The AA genotype was also greater in the COPD cohort compared with resistant smokers (OR 1.4, P=0.12) a trend consistent with a susceptibility role (see Table 3a).
In the analysis of the −133 G/C promoter polymorphism of the IL18 gene, the C allele and CC genotype were found to be significantly greater in the COPD cohort compared to the controls (OR=1.36, P=0.05 and OR=1.44, P=0.06 respectively) consistent with a susceptibility role. The CC genotype was also greater in the COPD cohort compared with resistant smokers a trend consistent with a susceptibility role (see Table 3b).
In the analysis of the −675 4G/5G promoter polymorphism of the plasminogen activator inhibitor gene, the 5G allele and 5G5G genotype were found to be significantly greater in the COPD cohort compared to the resistant smoker cohort (OR=1.33, P=0.05 and OR=1.55, P=0.08) consistent with a susceptibility role. The greater frequency of the 5G5G in COPD compared to the blood donor cohort also suggests that the 5G5G genotype is associated with susceptibility (see Table 4).
In the analysis of the 298 Asp/Glu (T/G) polymorphism of the nitric oxide synthase (NOS3) gene, the TT genotype was found to be significantly greater in the resistant smoker cohort compared to the blood donor cohort (OR=2.2, P=0.03) consistent with a protective role. (see Table 5).
In the analysis of the Lys 420 Thr (A/C) polymorphism of the Vitamin D binding protein gene, the A allele and AA/AC genotype were found to be greater in the resistant smoker cohort compared to the COPD cohort (OR=1.34, P=0.05 and OR=1.39, P=0.10 respectively) consistent with a protective role. (see Table 6a).
In the analysis of the Glu 416 Asp (T/G) polymorphism of the Vitamin D binding protein gene, the T allele and TT/TG genotype were found to be greater in the resistant smoker cohort compared to the blood donor cohort cohort (OR=1.27, P=0.10 and OR=1.53, P=0.06 respectively) consistent with a protective role. (see Table 6b).
In the analysis of the Ile 105 Val (A/G) polymorphism of the glutathione S transferase P gene, the A allele and AA genotype were found to be greater in the resistant smoker cohort compared to the blood donor cohort (OR=1.34, P=0.05 and OR=1.45, P=0.07 respectively) consistent with a protective role. (see Table 7).
In the analysis of the 874 A/T polymorphism of the interferon-γ gene, the AA genotype was found to be significantly greater in the COPD cohort compared to the controls (OR-1.5, P=0.08) consistent with a susceptibility role. (see Table 8).
In the analysis of the Arg 130 Gln (G/A) polymorphism of the Interleukin 13 gene, the AA genotype was found to be greater in the resistant smoker cohort compared to the blood donor cohort (OR=2.94, P=0.09) consistent with a protective role. (see Table 9a).
In the analysis of the −1055 (C/T) polymorphism of the Interleukin 13 gene, the TT genotype was found to be greater in the COPD cohort compared to the resistant cohort (OR=6.03, P=0.03) consistent with a susceptibility role. (see Table 9b).
In the analysis of the α1-antitrypsin S polymorphism, the S allele and MS/SS genotype was found to be greater in the resistant smokers compared to COPD cohort (OR=2.42, P=0.01) consistent with a protective role (Table 10).
In the analysis of the +489 G/A polymorphism of the Tissue Necrosis Factor α gene, the A allele and the AA and AG genotypes were found to be greater in the COPD cohort compared to the controls (OR=1.57, P=0.11) consistent with a susceptibility role (see Table 11a). Conversely, the GG genotype was found to be greater in the resistant smoker cohort, consistent with a protective role (see Table 11a).
In the analysis of the −308 G/A polymorphism of the Tissue Necrosis Factor α gene, the GG genotype was found to be greater in the resistant smoker cohort compared to the COPD cohort (OR=0.77, P=0.20) consistent with a protective role. (see Table 11b). Conversely, the A allele and the AA and AG genotypes were found to be greater in the COPD cohort (OR=1.3, P=0.20), consistent with a susceptibility role (see Table 11b).
In the analysis of the C89Y A/G polymorphism of the SMAD3 gene, the AA and AG genotypes were found to be greater in the resistant smoker cohort compared to the COPD cohort (OR=0.26, P=0.07) consistent with a protective role. (see Table 12). Conversely, the GG genotype was found to be greater in the COPD cohort, consistent with a susceptibility role (see Table 12).
In the analysis of the E469K A/G polymorphism of the Intracellular adhesion molecule 1 gene, the G allele and the GG genotype were found to be greater in the COPD cohort compared to the controls (OR=1.3, P=0.09 and OR=1.6, P=0.07, respectively) consistent with a susceptibility role (see Table 13).
In the analysis of the Gly 881Arg G/C polymorphism of the Caspase (NOD2) gene, the CC and CG genotypes were found to be greater in the COPD cohort compared to the controls (OR=3.2, P=0.11) consistent with a susceptibility role (see Table 14).
In the analysis of the 161 G/A polymorphism of the Mannose binding lectin 2 gene, the GG genotype was found to be greater in the resistant smoker cohort compared to the COPD cohort (OR=0.53, P=0.003) consistent with a protective role. (see Table 15).
In the analysis of the −1903 G/A polymorphism of the Chymase 1 gene, the AA genotype was found to be greater in the resistant smoker cohort compared to the COPD cohort (OR=0.73, P=0.17) consistent with a protective role. (see Table 16).
In the analysis of the Arg 197 Gln G/A polymorphism of the N-Acetyl transferase 2 gene, the AA genotype was found to be greater in the resistant smoker cohort compared to the COPD cohort (OR=0.50, P=0.05) consistent with a protective role. (see Table 17).
In the analysis of the −511 A/G polymorphism of the Interleukin 1B gene, the GG genotype was found to be greater in the COPD cohort compared to the controls (OR=1.3, P=0.17) consistent with a susceptibility role (see Table 18).
In the analysis of the Tyr 113 His T/C polymorphism of the Microsomal epoxide hydrolase gene, the TT genotype was found to be greater in the COPD cohort compared to the controls (OR=1.5, P=0.06) consistent with a susceptibility role (see Table 19a).
In the analysis of the Arg 139 G/A polymorphism of the Microsomal epoxide hydrolase gene, the GG genotype was found to be greater in the resistant smoker cohort compared to the COPD cohort (OR=0.64, P=0.23) consistent with a protective role. (see Table 19b).
In the analysis of the −366 G/A polymorphism of the 5 Lipo-oxygenase gene, the AG and AA genotypes were found to be greater in the resistant smoker cohort compared to the COPD cohort (OR=0.60, P=0.12) consistent with a protective role. (see Table 20). Conversely, the GG genotype was found to be greater in the COPD cohort, consistent with a susceptibility role (see Table 20).
In the analysis of the HOM T2437C polymorphism of the Heat Shock Protein 70 gene, the CC and CT genotypes were found to be greater in the COPD cohort compared to the controls (OR=2.0, P=0.002) consistent with a susceptibility role (see Table 21). Conversely, the TT genotype was found to be greater in the resistant smoker cohort, consistent with a protective role (see Table 21).
In the analysis of the +13924 T/A polymorphism of the Chloride Channel Calcium-activated 1 gene, the AA genotype was found to be greater in the COPD cohort compared to the controls (OR=1.7, P=0.03) consistent with a susceptibility role (see Table 22).
In the analysis of the −159 C/T polymorphism of the Monocyte differentiation antigen CD-14 gene, the CC genotype was found to be greater in the COPD cohort compared to the controls (OR=1.4, P=0.15) consistent with a susceptibility role (see Table 23).
In the analysis of the Exon 1 +49 C/T polymorphism of the Elafin gene, the T allele and the CT and TT genotypes were found to be greater in the resistant smoker cohort compared to the COPD cohort (OR=0.69, P=0.17, OR=0.70, P=0.24, respectively) consistent with a protective role. (see Table 24).
In the analysis of the Gln 27 Glu C/G polymorphism of the β2-adrenergic receptor gene, the GG genotype was found to be greater in the resistant smoker cohort and the blood donor controls compared to the COPD cohort (OR=0.74, P=0.23, OR=0.69, P=0.14, respectively) consistent with a protective role. (see Table 25).
In the analysis of the −1607 1G/2G promoter polymorphism of the MMP1 gene, the 1G allele and 1G1G/1G2G genotypes were found to be significantly greater in the resistant smoker cohort compared to the COPD cohort (OR=0.73, p=0.03 and OR=0.55, p=0.009), consistent with a protective role. The greater frequency of the 1G1G in the resistant group compared to the blood donor cohort also suggests that the 1G allele is protective (see Table 26).

It is accepted that the disposition to chronic obstructive lung diseases (eg. emphysema and COPD) is the result of the combined effects of the individual's genetic makeup and their lifetime exposure to various aero-pollutants of which smoking is the most common. Similarly it is accepted that COPD encompasses several obstructive lung diseases and characterised by impaired expiratory flow rates (eg FEV1). The data herein suggest that several genes can contribute to the development of COPD. A number of genetic mutations working in combination either promoting or protecting the lungs from damage can be involved in elevated resistance or susceptibility.

From the analyses of the individual polymorphisms, 19 protective genotypes were identified and analysed for their frequencies in the smoker cohort consisting of resistant smokers and those with COPD. When the frequencies of resistant smokers and smokers with COPD were compared according to the presence of 0, 1 and 2+ protective genotypes (out of COX2 CC/CG, β2 adreno-receptor Arg 16 Gly AA, Interleukin-13 Arg 130 Gln AA, Nitic Oxide Synthase 3 298 TT and Vitamin D Binding Protein 420 AA/AC) significant differences were found (overall χ2=16.43, P=0.0003) suggesting that smokers with 2+protective genotypes had three times more likelihood of being resistant (OR=3.1, P=0.004) while those no protective genotypes were nearly twice as likely to have COPD (OR=1.74, P=0.006) (see Table 28). Examined another way, the chances of having COPD diminished from 63%, 55% to 32% in smokers with 0, 1 and 2+ of the protective genotypes tested for respectively. On analysis of a selection of the protective genotypes (out of COX2 CC/CG, NOS3 298 TT, VDBP-420 AA/AC, VDBP-416 TT/TG, GSTP1 AA, IL-13-140 AA, and α1-AT MS/SS), a significant difference in frequency of COPD versus resistance was found in those with 0 versus 1+ of the protective genotypes tested for (OR=2.82, P=0.0004)(see Table 30), showing a 2-3 fold increase in COPD in those with 0 of the protective genotypes tested for.

From the analyses of the individual polymorphisms, 17 susceptibility genotypes were identified and analysed for their frequencies in the smoker cohort consisting of resistant smokers and those with COPD. When the frequencies of resistant smokers and smokers with COPD were compared according to the presence of 0, 1 and 2+ susceptibility genotypes (out of Interleukin-18 105 AA, PAI-1-675 5G5G, Interleukin-13 −1055 TT and Interferon-γ −874 TT genotypes) significant differences were found (overall χ2=8.72, P=0.01) suggesting that smokers with 2+ of the susceptibility genotypes tested for had two times more likelihood of having COPD (OR=1.9, P=0.009) while those with none of the susceptibility genotypes tested for were 1.5 fold as likely to have COPD (OR=1.5, P=0.05) (see Table 29). Examined another way, the chances of having COPD increased from 49%, 55% to 68% in smokers with 0, 1 and 2+ of the susceptibility genotypes tested for respectively.

Thus, while single genotypes can be effective in predicting the risk that a subject can have in developing COPD, emphysema, or both, the strength of the correlation increased more than linearly (e.g., from a 6% increase to a 19% increase) with the presence of additional susceptibility genotypes and decreased more than linearly (e.g., from a 8% decrease to a 31% decrease) with the presence of additional protective genotypes. Thus, the anaysis of more than one genotype can be of great value, and the strength of the correlation appears greater than a simple linear increase due to two separate genotypes. As will be appreciated by one of skill in the art, and as discussed below, this not only allows one to obtain superior predictions of risk or the lack of risk, but also reveals that superior methods of treatment can involve enhancing multiple protective genotypes (for example, their protein products or the proteins they regulate) or inhibiting multiple susceptibility genotypes.

These findings indicate that the methods of the present invention can be predictive of COPD, emphysema, or both COPD and emphysema in an individual well before symptoms present. It is believed that the above examples are generally indicative of methods for not only obstructive lung disease in general, but COPD and emphysema in particular.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalising aberrant gene expression or gene product function. Additional examples of such treatment methods are discussed below.

As shown herein, the −765 G allele in the promoter of the gene encoding COX2 is associated with increased expression of the gene relative to that observed with the C allele. However, the C allele is protective with respect to the predisposition to or potential risk of developing COPD, emphysema, or both COPD and emphysema. Thus, a suitable therapy in subjects known to possess the −765 G allele can be the administration of an agent capable of reducing expression of the gene encoding COX2.

### Example 40

A patient with the −765G allele is identified, as described above. Following this, an agent capable of reducing the function of the gene encoding COX2, or the activity of COX2, is administered to the subject. An alternative suitable therapy can be the administration to such a subject of a COX2 inhibitor such as additional therapeutic approaches, gene therapy, RNAi.

As shown herein, the −133 C allele in the promoter of the gene encoding IL18 is associated with susceptibility to COPD, emphysema, or both COPD and emphysema. However, the −133 G allele in the promoter of the gene encoding IL18 is associated with increased IL18 levels. Thus, a suitable therapy in subjects known to possess the −133 C allele can be the administration of an agent capable of increasing expression of the gene encoding IL18.

### Example 41

A subject with the −133C allele in the promoter of the gene encoding IL18 will be identified and then an agent capable of increasing expression of the gene encoding IL18 will be provided to the subject (for example, additional IL18). Repeated doses will be administered as needed.

As shown herein the −675 5G5G genotype in the promoter of the plasminogen activator inhibitor gene is associated with susceptibility to COPD, emphysema, or both COPD and emphysema. The 5G allele is reportedly associated with increased binding of a repressor protein and decreased transcription of the gene. A suitable therapy can be the administration of an agent capable of decreasing the level of repressor and/or preventing binding of the repressor, thereby alleviating its downregulatory effect on transcription.

### Example 42

A subject with the −675 5G5G genotype is identified, as described above. The subject is administered an agent capable of preventing the binding of the repressor (for example, an antibody to the repressor). Thereby alleviating the repressor's downregulatory effect on transcription. An alternative therapy can include gene therapy, for example the introduction of at least one additional copy of the plasminogen activator inhibitor gene having a reduced affinity for repressor binding (for example, a gene copy having a −675 4G4G genotype).

### Example 43

In another example, a subject with two susceptibility genotypes is identified, as described above. The subject is administered agents that prevent or reduce the impact of the abnormality (compared to the function of the protective genotype or the genotype for the control group) resulting from both of the susceptibility genotypes.

Table 31 below presents representative examples of polymorphisms in linkage disequilibrium with the polymorphisms specified herein. Examples of such polymorphisms can be located using public databases, such as that available on the web, for example at world wide web dot hapmap dot org. Specified polymorphisms are indicated in the columns marked SNP NAME. Unique identifiers are indicated in the columns marked RS NUMBER.

TABLE 31
Polymorphisms reported to be in linkage disequilibrium
(unless stated) with the specified polymorphism.

| SNP NAME | RS NUMBER |
|---|---|
| | 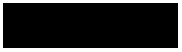 |
| | rs7527769 |
| | rs7550380 |
| | rs2206594 |
| | rs6687495 |
| | rs6681231 |
| | rs13376484 |
| | rs12064238 |
| | rs10911911 |
| | rs12743673 |
| | rs10911910 |
| | rs12743516 |
| | rs10911909 |
| | rs1119066 |
| | rs1119065 |
| | rs1119064 |
| | rs10798053 |
| | rs12409744 |
| | rs10911908 |
| | rs10911907 |
| | rs7416022 |
| | rs2745561 |
| | rs10911906 |
| | rs2734776 |
| | rs2734777 |
| | rs12084433 |
| | rs2734778 |
| | rs2745560 |
| | rs2223627 |
| | rs2383517 |
| | rs4295848 |
| | rs4428839 |
| | rs4609389 |
| | rs4428838 |
| | rs12131210 |
| | rs2179555 |
| | rs2143417 |
| | rs2143416 |
| | rs11583191 |
| | rs2383516 |
| | rs2383515 |
| | rs10911905 |
| | rs10911904 |
| | rs4648287 |
| | rs5272 |
| | rs4648288 |
| | rs5273 |
| | rs5274 |
| | rs3218625 |
| | rs4648289 |
| | rs4648290 |
| | rs1051896 |
| | rs5275 |
| | 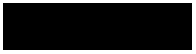 |
| | rs2082382 |
| | rs2082394 |
| | rs2082395 |
| | rs9325119 |
| | rs9325120 |
| | rs12189018 |
| | rs11168066 |
| | rs11959615 |
| | rs11958940 |
| | rs4705270 |
| | rs10079142 |
| | rs9325121 |
| | rs11746634 |
| | rs11168067 |
| | rs9325122 |
| | rs11957351 |
| | rs11948371 |
| | rs11960649 |
| | rs1432622 |
| | rs1432623 |
| | rs11168068 |
| | rs17778257 |
| | rs2400706 |
| | rs2895795 |
| | rs2400707 |
| | rs2053044 |
| | rs17108803 |
| | rs12654778 |
| | rs11168070 |
| | rs11959427 |
| | rs1042711 |
| | rs1801704 |
| 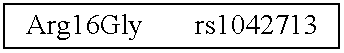 | |
| | rs1042714 |
| | rs1042717 |
| | rs1800888 |
| | rs1042718 |
| | rs3729943 |
| | rs12703107 |
| | rs6946340 |
| | rs6946091 |
| | rs6946415 |
| | rs10952296 |
| | rs13309715 |
| | rs10952297 |
| | rs7784943 |
| | rs11771443 |
| | rs2243310 |
| | rs1800783 |
| | rs3918155 |
| | rs3918156 |
| | rs2566519 |
| | rs3918157 |
| | rs3918158 |
| | rs3918159 |
| | rs2566516 |
| | rs3918225 |
| | rs3918160 |
| | rs1800779 |
| | rs2243311 |
| | rs3918161 |
| | rs10952298 |
| | rs2070744 |
| | rs3918226 |
| | rs3918162 |
| | rs3918163 |
| | rs3918164 |
| | rs3918165 |
| | rs1800781 |
| | rs13310854 |
| | rs13310763 |
| | rs2853797 |
| | rs13311166 |
| | rs13310774 |
| | rs2853798 |
| | rs11974098 |
| | rs3918166 |
| | rs3730001 |
| | rs3918167 |
| | rs3918168 |
| | rs3918169 |
| | rs3918170 |
| | rs3793342 |
| | rs3793341 |
| | rs1549758 |
| | rs1007311 |
| | rs9282803 |
| | rs8191438 |
| | rs8191439 |
| | rs8191440 |
| | rs8191441 |
| | rs1079719 |
| | rs1871041 |
| | rs4147581 |
| | rs8191444 |
| | rs8191445 |
| | rs2370143 |
| | rs8191446 |
| | rs3891249 |
| | rs8191447 |
| | rs12796085 |
| | rs8191448 |
| | rs762803 |
| | rs8191449 |
| 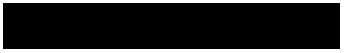 | |
| | rs4986948 |
| | rs675554 |
| | rs749174 |
| | rs8191450 |
| | rs743679 |
| | rs1799811 |
| | rs11553890 |
| | rs4986949 |
| | rs8191451 |
| | rs1871042 |
| | rs11553892 |
| | rs4891 |
| | rs6413486 |
| | rs5031031 |
| | rs947895 |
| | 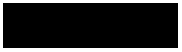 |
| | rs2069707 |
| | rs3814242 |
| | rs2069709 |
| | rs2069710 |
| | rs2069711 |
| | rs2069712 |
| 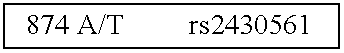 | |
| | rs2069713 |
| | rs1861494 |
| | rs2234685 |
| | rs1861493 |
| | rs2069714 |
| | rs2069715 |
| | rs2069716 |
| | rs2069717 |
| | rs1885065 |
| | rs1884548 |
| | rs1243167 |
| | rs17751614 |
| | rs1884549 |
| | rs1243168 |
| | rs17090693 |
| | rs17824597 |
| | 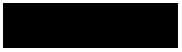 |
| | rs1799964 |
| | rs1800630 |
| | rs1799724 |
| 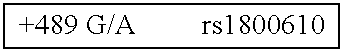 | |
| | rs3093662 |
| | rs3093664 |
| 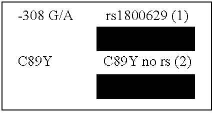 | |
| | rs1799969 |
| | rs5493 |
| | rs5030381 |
| | rs5494 |
| | rs3093033 |
| | rs5495 |
| | rs1801714 |
| | rs13306429 |
| | rs2071441 |
| | rs5496 |
| | rs5497 |
| | rs13306430 |
| 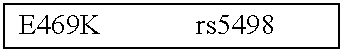 | |
| | rs5030400 |
| | rs2071440 |
| | rs5499 |
| | rs3093032 |
| | rs1057981 |
| | rs5500 |
| | rs5501 |
| | rs5030383 |
| | rs281436 |
| | rs923366 |
| | rs281437 |
| | rs3093030 |
| | rs5030384 |
| | rs5030385 |
| | rs3810159 |
| | rs281438 |
| | rs3093029 |
| | rs5743274 |
| | rs1861759 |
| | rs5743275 |
| | rs5743276 |
| | rs2066844 |
| | rs5743277 |
| | rs5743278 |
| | rs6413461 |
| | rs3813758 |
| | rs5743279 |
| | rs5743280 |
| | rs5743281 |
| | rs4785225 |
| | rs16948773 |
| | rs9931711 |
| | rs17313265 |
| | rs11646168 |
| | rs9925315 |
| | rs5743284 |
| | rs5743285 |
| | rs751271 |
| | rs748855 |
| | rs1861758 |
| | rs13332952 |
| | rs7198979 |
| | rs1861757 |
| | rs7203691 |
| | rs5743286 |
| | rs5743287 |
| | rs10521209 |
| 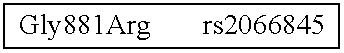 | |
| | rs5743289 |
| | rs8063130 |
| | rs2076756 |
| | rs12920425 |
| | rs12920040 |
| | rs12920558 |
| | rs12919099 |
| | rs12920721 |
| | rs2076755 |
| | rs5743290 |
| | rs5743291 |
| | rs11642651 |
| | rs1861756 |
| | rs749910 |
| | rs4990643 |
| | rs1077861 |
| | rs5743292 |
| | rs9921146 |
| | rs7820330 |
| | rs7460995 |
| | rs2087852 |
| | rs2101684 |
| | rs7011792 |
| | rs1390358 |
| | rs923796 |
| | rs4546703 |
| | rs4634684 |
| | rs2410556 |
| | rs11996129 |
| | rs4621844 |
| | rs11785247 |
| | rs1115783 |
| | rs1115784 |
| | rs1961456 |
| | rs1112005 |
| | rs11782802 |
| | rs973874 |
| | rs1495744 |
| | rs7832071 |
| | rs1805158 |
| | rs1801279 |
| | rs1041983 |
| | rs1801280 |
| | rs4986996 |
| | rs12720065 |
| | rs4986997 |
| | rs1799929 |
| 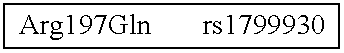 | |
| | rs1208 |
| | rs1799931 |
| | rs2552 |
| | rs4646247 |
| | rs971473 |
| | rs721398 |
| | 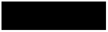 |
| | rs10169916 |
| | rs13009179 |
| | rs4849127 |
| | rs4849126 |
| | rs7558108 |
| | rs13032029 |
| | rs13013349 |
| | rs12623093 |
| | rs3087255 |
| | rs3087256 |
| | rs6721954 |
| | rs12621220 |
| | rs4584668 |
| | rs4238137 |
| | rs17612127 |
| | rs4147063 |
| | rs4147064 |
| | rs4147062 |
| | rs9315046 |
| | rs9506352 |
| | rs9670531 |
| | rs9671182 |
| | rs9315047 |
| | rs17690694 |
| | rs9652070 |
| | rs17074966 |
| | rs4387455 |
| | rs4254166 |
| | rs4075692 |
| | rs17690748 |
| | rs9671124 |
| | rs9671125 |
| | rs9741436 |
| | rs9578197 |
| | rs4769056 |
| | rs11147439 |
| | rs12721459 |
| | rs4769874 |
| | 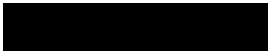 |
| | rs1043618 |
| | rs11576009 |
| | rs11557922 |
| | rs11576010 |
| | rs1008438 |
| | rs11576011 |
| | rs4713489 |
| | rs16867582 |
| | rs12526722 |
| | rs6933097 |
| | rs12213612 |
| | rs481825 |
| | rs7757853 |
| | rs7757496 |
| | rs9469057 |
| | rs12182397 |
| | rs16867580 |
| | rs2075799 |
| | rs482145 |
| | rs2227957 |
| 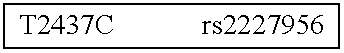 | |
| | rs2227955 |
| | rs5744345 |
| | rs1358825 |
| | rs2145410 |
| | rs2734695 |
| | rs5744346 |
| | rs5744347 |
| | rs100000105 |
| | rs5744349 |
| | rs4655913 |
| | rs1321696 |
| | rs5744352 |
| | rs11583355 |
| | rs100000106 |
| | rs1321695 |
| 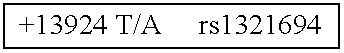 | |
| | rs2791514 |
| | rs2734696 |
| | rs5744354 |
| | rs2791513 |
| | rs2753332 |
| | rs2791512 |
| | rs2791511 |
| | rs2734697 |
| | 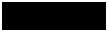 |
| | rs6877461 |
| | rs3822356 |
| | rs6877437 |
| | rs12153256 |
| | rs11554680 |
| | rs12109040 |
| | rs12517200 |
| | rs5744430 |
| | rs5744431 |
| | rs100000092 |
| | rs5744433 |
| | rs100000093 |
| | rs4912717 |
| | rs100000094 |
| | rs100000095 |
| | rs100000096 |
| | rs6864930 |
| | rs100000097 |
| | rs6864583 |
| | rs6864580 |
| | rs6889418 |
| | rs6889416 |
| | rs5744440 |
| | rs5744441 |
| | rs5744442 |
| | rs11168067 |
| | rs9325122 |
| | rs11957351 |
| | rs11948371 |
| | rs11960649 |
| | rs1432622 |
| | rs1432623 |
| | rs11168068 |
| | rs17778257 |
| | rs2400706 |
| | rs2895795 |
| | rs2400707 |
| | rs2053044 |
| | rs17108803 |
| | rs12654778 |
| | rs11168070 |
| | rs11959427 |
| | rs1042711 |
| | rs1801704 |
| | rs1042713 |
| 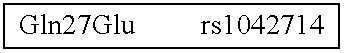 | |
| | rs1042717 |
| | rs1800888 |
| | rs1042718 |
| 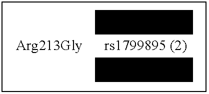 | |
| | rs1529717 |
| | rs1046909 |
| | rs2241712 |
| | rs2241713 |
| | rs2241714 |
| | rs11673525 |
| | rs2873369 |
| | rs11083617 |
| | rs11083616 |
| | rs4803458 |
| | rs11670143 |
| | rs1982072 |
| | rs11668109 |
| | rs13345981 |
| | rs11666933 |
| | rs11466310 |
| | rs11466311 |
| | rs2317130 |
| | rs4803457 |
| | rs3087453 |
| | rs1800820 |
| | rs1054797 |
| | rs6073964 |
| | rs6073985 |
| | rs8121146 |
| | rs6032620 |
| | rs11698788 |
| | rs6032621 |
| | rs6065912 |
| | rs6104417 |
| | rs3848720 |
| | rs13040272 |
| | rs6104418 |
| | rs3848721 |
| | rs3848722 |
| | rs6104419 |
| | rs4810482 |
| | rs3761157 |
| | rs3761158 |
| | rs3761159 |
| | rs8113877 |
| | rs6065913 |
| | rs6104420 |
| | rs6104421 |
| | rs3918240 |
| | rs6104422 |
| | rs3918278 |
| | rs3918241 |
| 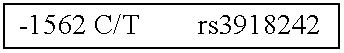 | |
| | rs3918243 |
| | rs3918279 |
| | rs3918280 |
| | rs4578914 |
| | rs6017724 |
| | rs3918244 |
| | rs3918245 |
| | rs6130992 |
| | rs3918247 |
| | rs3918248 |
| | rs3918249 |
| | rs6104423 |
| | rs6104424 |
| | rs6104425 |
| | rs6104426 |
| | rs3918250 |
| | rs1805089 |
| | rs3918251 |
| | rs13040572 |
| | rs13040580 |
| | rs3918252 |
| | rs8125581 |
| | rs668491 2 |
| | rs2745559 |
| | rs12042763 |
| | rs4648250 |
| | rs4648251 |
| | rs2223626 |
| | rs689462 |
| | rs4648253 |
| | rs689465 |
| | rs12027712 |
| | rs689466 |
| | rs2745558 |
| | rs3918304 |
| | rs20415 |
| | rs20416 |
| | rs4648254 |
| | rs11567815 |
| −765G>C | rs20417 |
| | rs4648256 |
| | rs20419 |
| | rs2734779 |
| | rs20420 |
| | rs20422 |
| | rs20423 |
| | rs5270 |
| | rs20424 |
| | rs5271 |
| | rs4648257 |
| | rs11567819 |
| | rs3134591 |
| | rs3134592 |
| | rs20426 |
| | rs4648258 |
| | rs11567820 |
| | rs2745557 |
| | rs11567821 |
| | rs4648259 |
| | rs4648260 |
| | rs4648261 |
| | rs4648262 |
| | rs11567822 |
| | rs11567823 |
| | rs2066824 |
| | rs20427 |
| | rs1042719 |
| | rs3729944 |
| | rs3730182 |
| | rs1042720 |
| | rs6879202 |
| | rs3777124 |
| | rs1803051 |
| | rs8192451 |
| | rs4987255 |
| | rs3177007 |
| | rs1126871 |
| | rs6885272 |
| | rs6889528 |
| | rs4521458 |
| | rs10463409 |
| | rs7702861 |
| | IL-I8 SNPs |
| | rs187238 |
| | rs5744228 |
| | rs360718 |
| | rs360717 |
| | rs5744229 |
| | rs100000353 |
| | rs5744231 |
| | rs5744232 |
| | rs7106524 |
| | rs189667 |
| | rs12290658 |
| | rs12271175 |
| | rs11606049 |
| | rs360716 |
| | rs360715 |
| | rs360714 |
| | rs2043055 |
| | rs5744233 |
| | rs795467 |
| | rs12270240 |
| | rs100000354 |
| | rs4937113 |
| | rs100000355 |
| | rs360723 |
| | rs5744237 |
| | rs5744238 |
| | rs5744239 |
| | rs7932965 |
| | rs11214103 |
| | rs5744241 |
| | rs5744242 |
| | rs5744243 |
| | rs9282804 |
| Asp298Glu | rs1799983 |
| | VDBP SNPs |
| | rs222035 |
| | rs222036 |
| | rs16846943 |
| | rs7668653 |
| | rs1491720 |
| | rs16845007 |
| | rs17830803 |
| Glu416Asp | rs7041 |
| Lys420Thr | rs4588 |
| | rs3737553 |
| | rs9016 |
| | rs1352846 |
| | rs222039 |
| | rs3775154 |
| | rs222040 |
| | rs843005 |
| | rs222041 |
| | rs7672977 |
| | rs705121 |
| | rs11723621 |
| | rs2298850 |
| | rs705120 |
| | rs2298851 |
| | rs844806 |
| | rs1491709 |
| | rs705119 |
| | rs6845925 |
| | rs12640255 |
| | rs12644050 |
| | rs6845869 |
| | rs12640179 |
| | rs222042 |
| | rs3187319 |
| | rs222043 |
| | rs842999 |
| | rs222044 |
| | rs222045 |
| | rs16846912 |
| | rs222046 |
| | rs705118 |
| | rs222047 |
| | rs13142062 |
| | rs843000 |
| | rs3755967 |
| | rs1491710 |
| | rs2282678 |
| | rs2069718 |
| | rs3087272 |
| | rs2069719 |
| | rs9282708 |
| | rs2069720 |
| | rs1042274 |
| | rs2069721 |
| | rs2069734 |
| | rs2069722 |
| | rs2234687 |
| | rs7957366 |
| | rs2069723 |
| | rs2069724 |
| | rs2069725 |
| | rs4394909 |
| | rs2069726 |
| | rs2069727 |
| | IL-13 SNPs |
| −1055 C/T | rs1800925 |
| | rs11575055 |
| | rs2069755 |
| | rs2069741 |
| | rs2069742 |
| | rs2069743 |
| | rs2069756 |
| | rs3212142 |
| | rs2066960 |
| | rs1295687 |
| | rs3212145 |
| | rs2069744 |
| | rs2069745 |
| | rs2069746 |
| | rs2069747 |
| | rs2069748 |
| | rs1295686 |
| Arg130Gln | rs20541 |
| | rs2069749 |
| | rs1295685 |
| | rs848 |
| | rs2069750 |
| | rs847 |
| | a1-antitrypsin |
| | SNPs |
| | rs709932 |
| | rs11558261 |
| | rs20546 |
| | rs11558263 |
| | F1028580 |
| | rs7145770 |
| | rs2239652 |
| | rs2735442 |
| | rs2569693 |
| | rs281439 |
| | rs281440 |
| | rs2569694 |
| | rs11575073 |
| | rs2569695 |
| | rs2075741 |
| | rs11575074 |
| | rs2569696 |
| | rs2735439 |
| | rs2569697 |
| | rs2075742 |
| | rs2569698 |
| | rs11669397 |
| | rs901886 |
| | rs885742 |
| | rs2569699 |
| | rs1056538 |
| | rs11549918 |
| | rs2569700 |
| | rs2228615 |
| | rs2569701 |
| | rs2569702 |
| | rs2735440 |
| | rs2569703 |
| | rs10418913 |
| | rs1056536 |
| | rs2569704 |
| | rs11673661 |
| | rs2569705 |
| | rs10402760 |
| | rs2569706 |
| | rs2569707 |
| | rs2735441 |
| | rs2436545 |
| | rs2436546 |
| | rs2916060 |
| | rs2916059 |
| | rs2916058 |
| | rs2569708 |
| | rs12972990 |
| | rs735747 |
| | rs885743 |
| | NOD2 SNPs |
| | rs4785224 |
| | rs5743261 |
| | rs5743262 |
| | rs5743263 |
| | rs11645386 |
| | rs7187857 |
| | rs8061960 |
| | rs5743294 |
| | rs2357791 |
| | rs7359452 |
| | rs7203344 |
| | rs5743295 |
| | rs5743296 |
| | rs3135499 |
| | rs5743297 |
| | rs5743298 |
| | rs5743299 |
| | rs3135500 |
| | rs5743300 |
| | rs8056611 |
| | rs2357792 |
| | rs12600253 |
| | rs12598306 |
| | rs7205423 |
| | rs718226 |
| | MBL2 SNPs |
| | rs7899547 |
| | rs10824797 |
| | rs11003131 |
| | rs930506 |
| | rs930505 |
| | rs11003130 |
| | rs2384044 |
| | rs2384045 |
| | rs5027257 |
| | rs2384046 |
| | rs12263867 |
| | rs11003129 |
| | rs12221393 |
| | rs2165811 |
| | rs12782244 |
| | rs11003128 |
| | rs17664818 |
| | rs7475766 |
| | rs10824796 |
| | rs16933417 |
| | rs2165810 |
| | rs11003127 |
| | rs3925313 |
| | rs7094151 |
| | rs7071882 |
| | rs12264958 |
| | rs11003126 |
| | rs7596849 |
| | rs4848306 |
| | rs3087257 |
| | rs7556811 |
| | rs7556903 |
| | rs6743438 |
| | rs6743427 |
| | rs6761336 |
| | rs6761335 |
| | rs6743338 |
| | rs6761245 |
| | rs6761237 |
| | rs6743330 |
| | rs6743326 |
| | rs6743322 |
| | rs6761220 |
| | rs6761218 |
| | rs5021469 |
| | rs6710598 |
| | rs1143623 |
| | rs1143624 |
| | rs2708920 |
| | rs1143625 |
| | rs2853545 |
| | rs2708921 |
| | rs1143626 |
| | rs3087258 |
| C-511T | rs16944 |
| | rs3917346 |
| | rs4986962 |
| | rs1143627 |
| | MEH SNPs |
| Tyr113His | rs1051740 (2) |
| His139Arg | rs2234922 (2) |
| | ALOX5AP SNPs |
| | rs4076128 |
| | rs9508830 |
| | rs4073259 |
| | rs4073260 |
| | rs11616333 |
| | rs4073261 |
| | rs4075474 |
| | rs4075473 |
| | rs9670115 |
| | rs9315042 |
| | rs3809376 |
| | rs12877064 |
| | rs9508831 |
| | rs9670503 |
| | rs2075800 |
| | CLCA1 SNPs |
| | rs2791519 |
| | rs2791518 |
| | rs5744302 |
| | rs1321697 |
| | rs2753338 |
| | rs2791517 |
| | rs5744303 |
| | rs2734706 |
| | rs2753345 |
| | rs2753347 |
| | rs2753348 |
| | rs2753349 |
| | rs5744304 |
| | rs5744305 |
| | rs1358826 |
| | rs2753359 |
| | rs5744306 |
| | rs2734711 |
| | rs5744307 |
| | rs2734712 |
| | rs2753361 |
| | rs2753364 |
| | rs1555389 |
| | rs2753365 |
| | rs100000100 |
| | rs100000101 |
| | rs5744310 |
| | rs5744311 |
| | rs5744312 |
| | rs4656114 |
| | rs5744313 |
| | rs2753367 |
| | rs4656115 |
| | rs2734713 |
| | rs5744314 |
| | rs5744315 |
| | rs5744316 |
| | rs5744317 |
| | rs5744318 |
| | rs926063 |
| | rs5744319 |
| | rs5744320 |
| | rs5744321 |
| | rs5744322 |
| | rs5744323 |
| | rs5744324 |
| | rs2791516 |
| | rs5744443 |
| | rs5744444 |
| | rs3138074 |
| | rs13166911 |
| | rs2563310 |
| | rs2569193 |
| | rs2569192 |
| | rs5744446 |
| | rs5744447 |
| | rs5744448 |
| | rs3138076 |
| | rs12519656 |
| | rs5744449 |
| | rs2915863 |
| | rs3138078 |
| | rs6875483 |
| | rs2569191 |
| | rs5744451 |
| | rs5744452 |
| | rs100000098 |
| | rs17118968 |
| | rs5744455 |
| −159 C/T | rs2569190 |
| | rs2569189 |
| | rs2563303 |
| | rs3138079 |
| | rs2228049 |
| | rs13763 |
| | rs11556179 |
| | rs4914 |
| | Elafin SNPs |
| | rs2868237 |
| | rs4632412 |
| | rs7347427 |
| | rs6032032 |
| | rs10854230 |
| | rs7347426 |
| | rs8183548 |
| | rs6104047 |
| | rs6513967 |
| | rs13038813 |
| | rs8118673 |
| | rs7346463 |
| | rs7362841 |
| | rs13042694 |
| | rs13038342 |
| | rs7363327 |
| | rs6073668 |
| | rs13044826 |
| | rs1800468 |
| | rs4987025 |
| | rs1800469 |
| | rs11466314 |
| | rs12977628 |
| | rs12977601 |
| | rs12985978 |
| | rs11466315 |
| | rs11551223 |
| | rs11551226 |
| | rs11466316 |
| | rs13306706 |
| | rs13306707 |
| | rs13306708 |
| | rs9282871 |
| Leu10Pro | rs1982073 |
| | rs1800471 |
| | rs13447341 |
| | rs11466318 |
| | rs12976890 |
| | rs12978333 |
| | rs10420084 |
| | rs10418010 |
| | rs12983775 |
| | rs12462166 |
| | rs2241715 |
| | rs9749548 |
| | rs7258445 |
| | rs11466320 |
| | rs11466321 |
| | rs8108052 |
| | rs6508976 |
| | rs8108632 |
| | rs11466324 |
| | rs2241716 |
| | rs2241717 |
| | rs2288873 |
| | rs12973435 |
| | rs2014015 |
| | rs1989457 |
| | rs10406816 |
| | rs8102918 |
| | rs4803455 |
| | MMPI SNPs |
| | rs529381 |
| | rs1144396 |
| | rs504875 |
| | rs526215 |
| | rs12280880 |
| | rs8125587 |
| | rs3918253 |
| | rs2274755 |
| | rs2664538 |
| | rs3918254 |
| | rs6130993 |
| | rs3918255 |
| | rs2236416 |
| | rs6130994 |
| | rs3918256 |
| | rs3918281 |
| | rs3787268 |
| | rs3918257 |
| | rs6017725 |
| | rs6032623 |
| | rs3918258 |
| | rs2250889 |
| | rs3918259 |
| | rs3918260 |
| | rs13969 |
| | rs6104427 |
| | rs6104428 |
| | rs2274756 |
| | rs6017726 |
| | rs3918261 |
| | rs6032624 |
| | rs3918262 |
| | rs3918263 |
| | rs3918264 |
| | rs6130995 |
| | rs6130996 |
| | rs3918265 |
| | rs3918266 |
| | rs3918267 |
| | rs6073987 |
| | rs6073988 |
| | rs3918282 |
| | rs1802909 |
| | rs13925 |
| | rs20544 |
| | rs1056628 |
| | rs1802908 |
| | rs2664517 |
| | rs9509 |
| | rs3918268 |
| | rs3918269 |
| | rs3918270 |
| | MMP12 SNPs |
| −82 A/G | rs2276109 (2) |
| | rs5277 |
| | rs2066823 |
| | rs4648263 |
| | rs4987012 |
| | rs20428 |
| | rs20429 |
| | rs4648264 |
| | rs4648265 |
| | rs4648266 |
| | rs4648267 |
| | rs11567824 |
| | rs4648268 |
| | rs4648269 |
| | rs4648270 |
| | rs12759220 |
| | rs20430 |
| | rs4648271 |
| | rs11567825 |
| | rs4648273 |
| | rs16825748 |
| | rs4648274 |
| | rs16825745 |
| | rs20432 |
| | rs20433 |
| | rs3218622 |
| | rs2066826 |
| | rs5278 |
| | rs4648276 |
| | rs20434 |
| | rs3218623 |
| | rs3218624 |
| | rs5279 |
| | rs4648278 |
| | rs13306034 |
| | rs2853803 |
| | rs4648279 |
| | rs4648281 |
| | rs4648282 |
| | rs11567826 |
| | rs4648283 |
| | rs4648284 |
| | rs4648285 |
| | rs11567827 |
| | rs4648286 |
| | rs5744244 |
| | rs360722 |
| | rs5023207 |
| | rs5744246 |
| | rs5744247 |
| −133 C/G | rs360721 |
| | rs4988359 |
| | rs12721559 |
| | rs5744248 |
| | rs5744249 |
| | rs5744250 |
| | rs5744251 |
| | rs100000356 |
| | rs1834481 |
| | rs17215057 |
| | rs5744253 |
| | rs5744254 |
| | rs5744255 |
| | rs5744256 |
| | rs5744257 |
| | rs360720 |
| | rs5744258 |
| | rs5744259 |
| | rs5744260 |
| | rs5744261 |
| 105 A/C | rs549908 |
| PAI-1 SNPs | |
| | rs6465787 |
| | rs7788533 |
| | rs6975620 |
| | rs6956010 |
| | rs12534508 |
| | rs4729664 |
| | rs2527316 |
| | rs2854235 |
| | rs10228765 |
| | rs2854225 |
| | rs2854226 |
| | rs2227707 |
| | rs2227631 |
| −675 4G15G | No rs |
| | NOS3 SNPs |
| | rs2373962 |
| | rs2373961 |
| | rs6951150 |
| | rs13238512 |
| | rs10247107 |
| | rs10276930 |
| | rs10277237 |
| | rs2282679 |
| | rs2282680 |
| | rs705117 |
| | rs2070741 |
| | rs2070742 |
| | rs6821541 |
| | rs222048 |
| | rs432031 |
| | rs432035 |
| | rs222049 |
| | rs222050 |
| | rs12510584 |
| | rs17467825 |
| | GSTPI SNPs |
| | rs656652 |
| | rs625978 |
| | rs6591251 |
| | rs12278098 |
| | rs612020 |
| | rs12284337 |
| | rs12574108 |
| | rs6591252 |
| | rs597717 |
| | rs688489 |
| | rs597297 |
| | rs6591253 |
| | rs6591254 |
| | rs7927381 |
| | rs7940813 |
| | rs593055 |
| | rs7927657 |
| | rs614080 |
| | rs7941395 |
| | rs7941648 |
| | rs7945035 |
| | rs2370141 |
| | rs2370142 |
| | rs7949394 |
| | rs7949587 |
| | rs6591255 |
| | rs8191430 |
| | rs6591256 |
| | rs8191431 |
| | rs8191432 |
| | rs7109914 |
| | rs4147580 |
| | rs8191436 |
| | rs8191437 |
| | rs17593068 |
| | rs7145047 |
| | rs7141735 |
| | rs11558264 |
| | rs6647 |
| | rs8350 |
| | rs2230075 |
| | rs1049800 |
| S allele | rs17580 |
| | rs2854258 |
| | rs2753937 |
| | rs2749547 |
| | rs1243162 |
| | rs2753938 |
| | rs2070709 |
| | rs17090719 |
| | rs11846959 |
| | rs1802962 |
| | rs2749521 |
| | rs2753939 |
| | rs1802959 |
| | rs1802961 |
| | rs1050469 |
| Z allele | no rs |
| | rs1050520 |
| | rs12077 |
| | rs12233 |
| | rs13170 |
| | rs1303 |
| | rs1802960 |
| | rs1243163 |
| | rs2073333 |
| | rs1243164 |
| | rs7144409 |
| | rs7142803 |
| | rs1243165 |
| | rs1051052 |
| | rs1243166 |
| | rs11628917 |
| | rs11832 |
| | rs9944155 |
| 1237 G/A | rs11568814 |
| | rs877081 |
| | rs877082 |
| | rs877083 |
| | rs877084 |
| | rs875989 |
| | rs9944117 |
| | rs1884546 |
| | rs1884547 |
| | rs8046608 |
| | rs5743264 |
| | rs5743266 |
| | rs2076752 |
| | rs5743267 |
| | rs8061316 |
| | rs8061636 |
| | rs16948754 |
| | rs7206340 |
| | rs2076753 |
| | rs2067085 |
| | rs16948755 |
| | rs2111235 |
| | rs2111234 |
| | rs7190413 |
| | rs7206582 |
| | rs8045009 |
| | rs6500328 |
| | rs7500036 |
| | rs8057341 |
| | rs12918060 |
| | rs7204911 |
| | rs7500826 |
| | rs4785449 |
| | rs12922299 |
| | rs11649521 |
| | rs13339578 |
| | rs17221417 |
| | rs13331327 |
| | rs11642482 |
| | rs11642646 |
| | rs17312836 |
| | rs5743268 |
| | rs5743269 |
| | rs5743270 |
| | rs12925051 |
| | rs12929565 |
| | rs13380733 |
| | rs13380741 |
| | rs11647841 |
| | rs10451131 |
| | rs2066842 |
| | rs5743271 |
| | rs7498256 |
| | rs5743272 |
| | rs5743273 |
| | rs2076754 |
| | rs2066843 |
| | rs1078327 |
| | rs1031101 |
| | rs10824795 |
| | rs10824794 |
| | rs920725 |
| | rs7916582 |
| | rs920724 |
| | rs16933335 |
| | rs11003125 |
| | rs7100749 |
| | rs11003124 |
| | rs7084554 |
| | rs7096206 |
| | rs11003123 |
| | rs11575988 |
| | rs11575989 |
| | rs7095891 |
| | rs4647963 |
| | rs8179079 |
| | rs5030737 |
| 161 G/A | rs1800450 |
| | rs1800451 |
| | rs12246310 |
| | rs12255312 |
| | rs11003122 |
| | rs1982267 |
| | rs1982266 |
| | rs4935047 |
| | rs4935046 |
| | rs10824793 |
| | rs1838066 |
| | rs1838065 |
| | rs930509 |
| | rs930508 |
| | rs930507 |
| | CMAI SNPs |
| | rs1956920 |
| | rs1956921 |
| −1903 G/A | rs1800875 |
| | rs1800876 |
| | rs3759635 |
| | rs1956922 |
| | rs1956923 |
| NAT2SNPs | |
| | rs11780272 |
| | rs2101857 |
| | rs13363820 |
| | rs6984200 |
| | rs13277605 |
| | rs9987109 |
| −366 G/A | rs9550373 |
| | rs11542984 |
| | rs4769055 |
| | rs17074937 |
| | rs9671065 |
| | rs9579645 |
| | rs9579646 |
| | rs4075131 |
| | rs4075132 |
| | rs9315043 |
| | rs9315044 |
| | rs4597169 |
| | rs9578037 |
| | rs9578196 |
| | rs4293222 |
| | rs10507391 |
| | rs12429692 |
| | rs4769871 |
| | rs4769872 |
| | rs4769873 |
| | rs12430051 |
| | rs9315045 |
| | rs9670278 |
| | rs4503649 |
| | rs9508832 |
| | rs9670460 |
| | rs3885907 |
| | rs3922435 |
| | rs9551957 |
| | rs12018461 |
| | rs9551958 |
| | rs10467440 |
| | rs12017304 |
| | rs9551959 |
| | rs11617473 |
| | rs11147438 |
| | rs10162089 |
| | rs9551960 |
| | rs9285075 |
| | rs12431114 |
| | rs4254165 |
| | rs4360791 |
| | rs17612031 |
| | rs3803277 |
| | rs3803278 |
| | rs12429469 |
| | rs17612099 |
| | rs9550576 |
| | rs4356336 |
| | rs2734714 |
| | rs6661730 |
| | rs2753377 |
| | rs2753378 |
| | rs2145412 |
| | rs2180762 |
| | rs1005569 |
| | rs5744325 |
| | rs5744326 |
| | rs1985554 |
| | rs1 985555 |
| | rs100000102 |
| | rs100000103 |
| | rs1969719 |
| | rs2390102 |
| | rs5744329 |
| | rs1407142 |
| | rs2753384 |
| | rs2753385 |
| | rs5744330 |
| | rs5744331 |
| | rs926064 |
| | rs926065 |
| | rs926066 |
| | rs926067 |
| | rs2753386 |
| | rs2180764 |
| | rs2734689 |
| | rs5744332 |
| | rs5744333 |
| | rs11161837 |
| | rs5744335 |
| | rs2038485 |
| | rs3765989 |
| | rs2734690 |
| | rs5744336 |
| | rs2734691 |
| | rs2734692 |
| | rs5744337 |
| | rs5744338 |
| | rs2734694 |
| | rs5744339 |
| | rs100000104 |
| | rs2791515 |
| | rs4656116 |
| | rs5744342 |
| | rs5744343 |
| | rs2180761 |
| | rs5744344 |
| | rs6032038 |
| | rs6032039 |
| | rs2267863 |
| | rs6124692 |
| +49 C/T | No rs |
| | rs17333103 |
| | rs17333180 |
| | rs1983649 |
| | rs16989785 |
| | rs17424356 |
| | rs6017500 |
| | rs6032040 |
| | rs6017501 |
| | rs2664581 |
| | rs17424474 |
| | rs17333381 |
| | rs1053826 |
| | rs2664533 |
| | rs1053831 |
| | rs2664520 |
| | rs2267864 |
| | rs13038355 |
| | rs13043296 |
| | rs13039213 |
| | rs6104049 |
| | rs13043503 |
| | rs6104050 |
| | rs17424578 |
| | rs17424613 |
| | rs6017502 |
| | rs6094101 |
| | rs6130778 |
| | rs6130779 |
| | rs6104051 |
| | rs6104052 |
| | ADBR2 SNPs |
| | rs2082382 |
| | rs2082394 |
| | rs2082395 |
| | rs9325119 |
| | rs9325120 |
| | rs12189018 |
| | rs11168066 |
| | rs11959615 |
| | rs11958940 |
| | rs4705270 |
| | rs10079142 |
| | rs9325121 |
| | rs11746634 |
| | rs542603 |
| | rs574939 |
| | rs573764 |
| | rs7102189 |
| | rs575727 |
| | rs552306 |
| | rs634607 |
| | rs12286876 |
| | rs12285331 |
| | rs519806 |
| | rs12283571 |
| | rs2839969 |
| | rs2000609 |
| | rs7125865 |
| | rs570662 |
| | rs11225427 |
| | rs484915 |
| | rs470307 |
| | rs2408490 |
| | rs12279710 |
| | rs685265 |
| | rs7107224 |
| | rs1155764 |
| | rs534191 |
| | rs509332 |
| | rs12283759 |
| | rs2105581 |
| | rs470206 |
| | rs533621 |
| −1607 G/GG | rs1799750 |
| | rs470211 |
| | rs470146 |
| | rs2075847 |
| | rs473509 |
| | rs498186 |
| | GSTMI |
| | polymorphism |
| Null | Null allele No rs (2) |
| | MMP9SNPs |
| | rs11696804 |
| | rs6104416 |
| | rs3933239 |
| | rs3933240 |
| | rs6094237 |
| | rs11697325 |
| | rs6130988 |
| | rs6073983 |
| | rs6130989 |
| | rs6130990 |
| | rs10211842 |
| | TIMP3 SNPs |
| | rs5754289 |
| | rs5754290 |
| | rs9606994 |
| | rs7285034 |
| | rs13433582 |
| | rs1962223 |
| | rs8137129 |
| | rs1807471 |
| | rs7290885 |
| | rs5749511 |
| | rs11703366 |
| | rs4990774 |
| −1296 T/C | rs9619311 |
| | rs2234921 |
| | rs2234920 |
| | rs16991235 |
| | rs4638893 |
| | rs12169569 |
| | rs5998639 |
| | rs7284166 |
| | rs5749512 |

(1 = no other SNPs reported to be in LD, 2 = no other SNPS reported to be in LD)

Suitable methods and agents for use in such therapy are well known in the art, and are discussed herein. However, as will be appreciated by one of skill in the art, given the identification of the present genotypes and their correlation with the risk of COPD, emphysema, or both, one of skill in the art will readily be able to determine the relevant downstream target (for example, a protein product that is controlled by the particular promoter) and manipulate it in a variety of ways (for example, antibodies, antisense RNA, siRNA, etc.). Additionally, as mentioned above, the ability to identify and then provide multiple approaches of treatment can have particular advantages, as noted above.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism. Additional information regarding the above methods and compositions can be found in U.S. patent application Ser. No. 10/479,525, filed Jun. 16, 2004; and PCT Application No. PCT/NZ02/00106, filed Jun. 5, 2002, which further designates New Zealand Application No. 512169, filed Jun. 5, 2001; New Zealand Application No. 513016, filed Jul. 17, 2001, and New Zealand Application No. 514275, filed Sep. 18, 2001, all of which are incorporated by reference in their entireties. Additional information can also be found in PCT application Nos. ______ and ______, filed May 10, 2006, entitled “Methods and Compsitions for Assessment of Pulmonary Function and Disorders” and “Methods of Analysis of Polymorphisms and Uses Thereof”, having Agent Reference Nos. 542813JBM and 542814JBM respectively, both of which are incorporated in their entirties by reference. PCT Application Agent Reference No. 542813JBM claims priority to: NZ application No. 539934, filed May 10, 2005; NZ application No. 541935, filed Aug. 19, 2005; and JP application No. 2005-360523, filed Dec. 14, 2005, all of which are incorporated by reference in their entireties. PCT Application Agent Reference No. 542814JBM claims priority to: NZ application No. 540249, filed May 20, 2005; and NZ application No. 541842, filed Aug. 15, 2005, all of which are incorporated in their entirties by reference. Additional information can also be found in U.S. pat. app. Ser. No. ______, filed concurrently with the instant application, entitled “Methods of Analysis of Polymorphisms and Uses Thereof,” attorney docket No; SGENZ.014AUS, incorporated in its entirety.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method includes the detection of the presence or absence of a susceptibility polymorphism which when present either upregulates or downregulates expression of the gene such that a state of such excess or deficit is the outcome, with those subjects in which the polymorphism is present being likely responders to treatment.

The present invention is directed to methods for assessing a subject's risk of developing chronic obstructive pulmonary disease (COPD), emphysema, or both COPD and emphysema. The methods include the analysis of polymorphisms herein shown to be associated with increased or decreased risk of developing COPD, emphysema, or both COPD and emphysema, or the analysis of results obtained from such an analysis. The use of polymorphisms herein shown to be associated with increased or decreased risk of developing COPD, emphysema, or both COPD and emphysema in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicant reserves the right to physically incorporate into this specification any and all materials and information from any such patents, publications, scientific articles, web sites, electronically available information, and other referenced materials or documents. Included in this is Waltenberg J. (2001. Pathophysiological basis of unstable coronary syndrome. Herz 26. Supp 1; 2-8.) incorporated in its entirety by reference.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” can be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably can be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by the Applicant.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed can be resorted to by those skilled in the art, and that such modifications and variations are considered to be within the scope of this invention as defined by the appended claims.

1. A method of determining a subject's risk of developing one or more obstructive lung diseases comprising analysing a sample from said subject for a presence or absence of one or more polymorphisms selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881 Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding Matrix Metalloproteinase 1 (MMP1), with reference to the 1G allele only; and
one or more polymorphisms which are in linkage disequilibrium with any one or more of these polymorphisms;
wherein the presence or absence of one or more of said polymorphisms is indicative of the subject's risk of developing one or more obstructive lung diseases selected from the group consisting of chronic obstructive pulmonary disease (COPD), emphysema, or both COPD and emphysema.

2. A method according to claim 1 wherein the presence of one or more of the polymorphisms is selected from the group consisting of:
the −765 CC or CG genotype in the promoter of the gene encoding COX2;
the +489 GG geneotype in the gene encoding TNFα;
the C89Y AA or AG geneotype in the gene encodoing SMAD3;
the 161 GG genotype in the gene encodoing MBL2;
the −1903 AA genotype in the gene encoding CMA1;
the Arg 197 Gln AA genotype in the gene encoding NAT2;
the −366 AA or AG genotype in the gene encoding ALOX5;
the HOM T2437C TT genotype in the gene encoding HSP 70;
the exon 1 +49 CT or TT genotype in the gene encoding Elafin; and
the −1607 1G1G or 1G2G genotype in the promoter of the gene encoding MMP1;
wherein the one or more polymorphism is indicative of a reduced risk of developing COPD, emphysema, or both COPD and emphysema.

3. A method according to claim 1 wherein the presence of one or more of the polymorphisms is selected from the group consisting of:
the 105 AA genotype in the gene encoding IL18;
the −133 CC genotype in the promoter of the gene encoding IL18;
the −675 5G5G genotype in the promoter of the gene encoding PAI-1;
the 874 TT genotype in the gene encoding IFN-γ;
the +489 AA or AG genotype in the gene encoding TNFα;
the C89Y GG genotype in the gene encoding SMAD3;
the E469K GG genotype in the gene encoding ICAM1;
the Gly 881 Arg GC or CC genotype in the gene encoding NOD2;
the −366 GG genotype in the gene encoding ALOX5;
the HOM T2437C CC or CT genotype in the gene encoding HSP 70;
the +13924 AA genotype in the gene encoding CLCA1; and
the −159 CC genotype in the gene encoding CD-14;
wherein the one or more polymorphism is indicative of an increased risk of developing COPD, emphysema, or both COPD and emphysema.

4. A method according to claim 1 wherein the method comprises analysing said sample for the presence or absence of one or more further polymorphisms selected from the group consisting of:
16 Arg/Gly in the gene encoding β₂ adrenergic receptor (ADBR);
130 Arg/Gln (G/A) in the gene encoding Interleukin 13 (IL13);
298 Asp/Glu (T/G) in the gene encoding nitric oxide synthase 3 (NOS3);
Ile 105 Val (A/G) in the gene encoding glutathione S transferase P (GST-P);
Glu 416 Asp (T/G) in the gene encoding Vitamin D binding protein (VDBP);
Lys 420 Thr (A/C) in the gene encoding VDBP;
−1055 C/T in the promoter of the gene encoding IL13;
−308 G/A in the promoter of the gene encoding TNFα;
−511 A/G in the promoter of the gene encoding Interleukin 1B (IL1B);
Tyr 113 His T/C in the gene encoding Microsomal epoxide hydrolase (MEH);
Arg 139 G/A in the gene encoding MEH;
Gln 27 Glu C/G in the gene encoding ADBR
−1607 1G/2G in the promoter of the gene encoding MMP1 (with reference to the 2G allele only);
−1562 C/T in the promoter of the gene encoding MMP9;
M1 null in the gene encoding GST-1;
1237 G/A in the 3′ region of the gene encoding α1-antitrypsin;
−82 A/G in the promoter of the gene encoding MMP12;
T→C within codon 10 of the gene encoding TGFβ;
760 C/G in the gene encoding SOD3;
−1296 T/C within the promoter of the gene encoding TIMP3;
the S mutation in the gene encoding α1-antitrypsin; and
one or more polymorphisms which are in linkage disequilibrium with one or more of these polymorphisms.

5. A method according to claim 4 wherein the polymorphism is selected from the group consisting of:
the −765 CC or CG genotype in the promoter of the gene encoding COX2;
the 130 Arg/Gln AA genotype in the gene encoding IL13;
the 298 Asp/Glu TT genotype in the gene encoding NOS3;
the Lys 420 Thr AA or AC genotype in the gene encoding VDBP;
the Glu 416 Asp TT or TG genotype in the gene encoding VDBP;
the Ile 105 Val AA genotype in the gene encoding GSTP-1;
the MS genotype in the gene encoding α1-antitrypsin;
the +489 GG geneotype in the gene encoding TNFα;
the −308 GG geneotype in the gene encoding TNFα;
the C89Y AA or AG geneotype in the gene encodoing SMAD3;
the 161 GG genotype in the gene encodoing MBL2;
the −1903 AA genotype in the gene encoding CMA1;
the Arg 197 Gln AA genotype in the gene encoding NAT2;
the His 139 Arg GG genotype in the gene encoding MEH;
the −366 AA or AG genotype in the gene encoding ALOX5;
the HOM T2437C TT genotype in the gene encoding HSP 70;
the exon 1 +49 CT or TT genotype in the gene encoding Elafin;
the Gln 27 Glu GG genotype in the gene encoding ADBR; and
the −1607 1G1G or 1G2G genotype in the promoter of the gene encoding MMP1;
wherein said polymorphism is indicative of a reduced risk of developing COPD, emphysema, or both COPD and emphysema

6. A method according to claim 4 wherein the polymorphism is selected from the group consisting of:
the 105 AA genotype in the gene encoding IL18;
the −133 CC genotype in the promoter of the gene encoding IL18;
the −675 5G5G genotype in the promoter of the gene encoding PAI-1;
the −1055 TT genotype in the promoter of the gene encoding IL13;
the 874 TT genotype in the gene encoding IFN-γ;
the +489 AA or AG genotype in the gene encoding TNFα;
the −308 AA or AG genotype in the gene encoding TNFα;
the C89Y GG genotype in the gene encoding SMAD3;
the E469K GG genotype in the gene encoding ICAM1;
the Gly 881 Arg GC or CC genotype in the gene encoding NOD2;
the −511 GG genotype in the gene encoding IL1B;
the Tyr 113 His TT genotype in the gene encoding MEH;
the −366 GG genotype in the gene encoding ALOX5;
the HOM T2437C CC or CT genotype in the gene encoding HSP 70;
the +13924 AA genotype in the gene encoding CLCA1; and
the −159 CC genotype in the gene encoding CD-14;
wherein said polymorphism is indicative of an increased risk of developing COPD, emphysema, or both COPD and emphysema.

7. A method of assessing a subject's risk of developing one or more obstructive lung diseases selected from COPD, emphysema, or both COPD and emphysema, said method comprising the steps:
(i) determining a presence or absence of at least one protective polymorphism associated with a reduced risk of developing COPD, emphysema, or both COPD and emphysema; and
(ii) in the absence of at least one protective polymorphisms, determining the presence or absence of at least one susceptibility polymorphism associated with an increased risk of developing COPD, emphysema, or both COPD and emphysema;
wherein the presence of one or more of said protective polymorphisms is indicative of a reduced risk of developing COPD, emphysema, or both COPD and emphysema, and the absence of at least one protective polymorphism in combination with the presence of at least one susceptibility polymorphism is indicative of an increased risk of developing COPD, emphysema, or both COPD and emphysema.

8. A method according to claim 7 wherein said at least one protective polymorphism is selected from the group consisting of:
−765 C in the promoter of the gene encoding COX2;
130 Arg/Gln A in the gene encoding IL13;
298 Asp/Glu T in the gene encoding NOS3;
Lys 420 Thr A in the gene encoding VDBP;
Glu 416 Asp T in the gene encoding VDBP;
Ile 105 Val A in the gene encoding GSTP-1;
the S mutation in the gene encoding α1-antitrypsin; +489 G in the gene encoding TNFα;
−308 G in the gene encoding TNFα;
C89Y A in the gene encoding SMAD3;
161 G in the gene encoding MBL2;
−1903 A in the gene encoding CMA1;
Arg 197 Gln A in the gene encoding NAT2;
His 139 Arg G in the gene encoding MEH;
−366 A in the gene encoding ALOX5;
HOM 2437 T in the gene encoding HSP 70;
exon 1 +49 T in the gene encodoing Elafin;
Gln 27 Glu G in the gene encoding ADBR; and
−1607 1G in the promoter of the gene encoding MMP1.

9. A method according to claim 7 wherein said at least one protective polymorphism is a genotype selected from the group consisting of:
the −765 CC or CG genotype in the promoter of the gene encoding COX2;
the 130 Arg/Gln AA genotype in the gene encoding IL13;
the 298 Asp/Glu TT genotype in the gene encoding NOS3;
the Lys 420 Thr AA or AC genotype in the gene encoding VDBP;
the Glu 416 Asp TT or TG genotype in the gene encoding VDBP;
the Ile 105 Val AA genotype in the gene encoding GSTP-1;
the MS genotype in the gene encoding α1-antitrypsin;
the +489 GG geneotype in the gene encoding TNFα;
the −308 GG geneotype in the gene encoding TNFα;
the C89Y AA or AG geneotype in the gene encodoing SMAD3;
the 161 GG genotype in the gene encodoing MBL2;
the −1903 AA genotype in the gene encoding CMA1;
the Arg 197 Gln AA genotype in the gene encoding NAT2;
the His 139 Arg GG genotype in the gene encoding MEH;
the −366 AA or AG genotype in the gene encoding ALOX5;
the HOM T2437C TT genotype in the gene encoding HSP 70;
the exon 1 +49 CT or TT genotype in the gene encoding Elafin;
the Gln 27 Glu GG genotype in the gene encoding ADBR; and
the −1607 1G1G or 1G2G genotype in the promoter of the gene encoding MMP1.

10. A method according to claim 7, said method further comprising determining a presence or absence of at least one further protective polymorphism selected from the group consisting of:
+760GG or +760CG within the gene encoding SOD3;
−1296TT within the promoter of the gene encoding TIMP3; and
CC (homozygous P allele) within codon 10 of the gene encoding TGFβ.

11. A method according to claim 7 wherein said at least one susceptibility polymorphism is a genotype selected from the group consisting of:
105 AA in the gene encoding Interleukin 18;
−133 CC in the promoter of the gene encoding Interleukin 18;
−675 5G5G in the promoter of the gene encoding plasminogen activator inhibitor 1;
−1055 TT in the promoter of the gene encoding Interleukin 13;
874 AA in the gene encoding interferon-γ;
+489 AA or AG in the gene encoding TNFα;
−308 AA or AG in the gene encoding TNFα;
C89Y GG in the gene encoding SMAD3;
E469K GG in the gene encoding ICAM1;
Gly 881 Arg GC or CC in the gene encoding NOD2;
−511 GG in the gene encoding IL1B;
Tyr 113 His TT in the gene encoding MEH;
−366 GG in the gene encoding ALOX5;
HOM T2437C CC or CT in the gene encoding HSP 70;
+13924 AA in the gene encoding CLCA1; or
−159 CC in the gene encoding CD-14.

12. A method according to claim 11 wherein said method comprises the step of determining the presence or absence of at least one further susceptibility polymorphism selected from the group consisting of:
−82 AA within the promoter of the gene encoding MMP12;
−1607 2G2G within the promoter of the gene encoding MMP1;
−1562CT or −1562TT within the promoter of the gene encoding MMP9; and
1237AG or 1237AA (Tt or tt allele genotypes) within the 3′ region of the gene encoding α1-antitrypsin.

13. A method according to claim 7 wherein the presence of two or more protective polymorphims irrespective of the presence of one or more susceptibility polymorphisms is indicative of reduced risk of developing COPD, emphysema, or both COPD and emphysema.

14. A method according to claim 7 wherein in the absence of a protective polymorphism the presence of one or more susceptibility polymorphisms is indicative of an increased risk of developing COPD, emphysema, or both COPD and emphysema.

15. A method according to claim 7 wherein the presence of two or more susceptibility polymorphisms is indicative of an increased risk of developing COPD, emphysema, or both COPD and emphysema.

16. A method of determining a subject's risk of developing chronic obstructive pulmonary disease (COPD) and/or emphysema, comprising analysing a sample from said subject for a presence of two or more polymorphisms selected from the group consisting of:
−765 C/G in the promoter of the gene encoding COX2;
105 C/A in the gene encoding IL18;
−133 G/C in the promoter of the gene encoding IL18;
−675 4G/5G in the promoter of the gene encoding PAI-1;
874 A/T in the gene encoding IFN-γ;
16Arg/Gly in the gene encoding ADBR;
130 Arg/Gln (G/A) in the gene encoding IL13;
298 Asp/Glu (T/G) in the gene encoding NOS3;
Ile 105 Val (A/G) in the gene encoding glutathione S transferase P (GST-P);
Glu 416 Asp (T/G) in the gene encoding VDBP;
Lys 420 Thr (A/C) in the gene encoding VDBP;
−1055 C/T in the promoter of the gene encoding IL13;
the S mutation in the gene encoding α1-antitrypsin;
+489 G/A in the gene encoding TNFα;
C89Y A/G in the gene encoding SMAD3;
E 469 K A/G in the gene encoding ICAM1;
Gly 881 Arg G/C in the gene encoding NOD2;
161 G/A in the gene encoding MBL2;
−1903 G/A in the gene encoding CMA1;
Arg 197 Gln G/A in the gene encoding NAT2;
−366 G/A in the gene encoding ALOX5;
HOM T2437C in the gene encoding HSP 70;
+13924 T/A in the gene encoding CLCA1;
−159 C/T in the gene encoding CD-14;
exon 1 +49 C/T in the gene encoding Elafin;
−308 G/A in the promoter of the gene encoding TNFα;
−511 A/G in the promoter of the gene encoding IL1B;
Tyr 113 His T/C in the gene encoding MEH;
Arg 139 G/A in the gene encoding MEH;
Gln 27 Glu C/G in the gene encoding ADBR; and
−1607 1G/2G in the promoter of the gene encoding MMP1 (with reference to the 1G allele only).

17. A method according to claim 1 wherein said method comprises the analysis of one or more epidemiological risk factors.

18. One or more nucleotide probes and/or primers for use in the method of any one of claims 1 to 17 wherein the one or more nucleotide probes and/or primers span, or are able to be used to span, the polymorphic regions of the genes in which the polymorphism to be analysed is present.

19. A nucleic acid microarray which comprises a substrate presenting nucleic acid sequences capable of hybridizing to nucleic acid sequences which encode one or more of the polymorphisms selected from the group defined in claim 1 or sequences complimentary thereto.

20. A method of determining a subject's risk of developing COPD, emphysema, or both COPD and emphysema, said method comprising:
(i) obtaining a result of one or more genetic tests of a sample from said subject; and (ii) analysing the result for a presence or absence of one or more polymorphisms selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881 Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding Matrix Metalloproteinase 1 (MMP1), with reference to the 1G allele only; and
one or more polymorphisms which are in linkage disequilibrium with any one or more of these polymorphisms;
wherein a result indicating the presence or absence of one or more of said polymorphisms is indicative of the subject's risk of developing COPD, emphysema, or both COPD and emphysema.

21. A method according to claim 20 wherein a result indicating the presence of one or more of the polymorphisms selected from the group consisting of:
the −765 CC or CG genotype in the promoter of the gene encoding COX2;
the +489 GG geneotype in the gene encoding TNFα;
the C89Y AA or AG geneotype in the gene encodoing SMAD3;
the 161 GG genotype in the gene encodoing MBL2;
the −1903 AA genotype in the gene encoding CMA1;
the Arg 197 Gln AA genotype in the gene encoding NAT2;
the −366 AA or AG genotype in the gene encoding ALOX5;
the HOM T2437C TT genotype in the gene encoding HSP 70;
the exon 1 +49 CT or TT genotype in the gene encoding Elafin; or
the −1607 1G1G or 1G2G genotype in the promoter of the gene encoding MMP1;
is indicative of a reduced risk of developing COPD, emphysema, or both COPD and emphysema.

22. A method according to claim 20 wherein a result indicating the presence of one or more of the polymorphisms selected from the group consisting of, the 105 AA genotype in the gene encoding IL18;
the −133 CC genotype in the promoter of the gene encoding IL18;
the −675 5G5G genotype in the promoter of the gene encoding PAI-1;
the 874 TT genotype in the gene encoding IFN-γ;
the +489 AA or AG genotype in the gene encoding TNFα;
the C89Y GG genotype in the gene encoding SMAD3;
the E469K GG genotype in the gene encoding ICAM1;
the Gly 881 Arg GC or CC genotype in the gene encoding NOD2;
the −366 GG genotype in the gene encoding ALOX5;
the HOM T2437C CC or CT genotype in the gene encoding HSP 70;
the +13924 AA genotype in the gene encoding CLCA1; and
the −159 CC genotype in the gene encoding CD-14;
is indicative of an increased risk of developing COPD, emphysema, or both COPD and emphysema.

23. (canceled)

24. (canceled)

25. A method treating a subject having an increased risk of developing COPD, emphysema, or both COPD and emphysema comprising the step of replicating, genotypically or phenotypically, a presence and/or functional effect of a protective polymorphism selected from the group defined in claim 8 in said subject.

26. A method of treating a subject having an increased risk of developing COPD, emphysema, or both COPD and emphysema, said subject having a detectable susceptibility polymorphism selected from the group defined in claim 11 which either upregulates or downregulates expression of a gene such that a physiologically active concentration of the expressed gene product is outside a range which is normal for the age and sex of the subject, said method comprising the step of restoring the physiologically active concentration of said product of gene expression to be within a range which is normal for the age and sex of the subject.

27. A method of treating a subject having an increased risk of developing COPD, emphysema, or both COPD and emphysema and for whom a presence of the GG genotype at the −765 C/G polymorphism present in a promoter of the gene encoding COX2 has been determined, said method comprising administering to said subject an agent capable of reducing COX2 activity in said subject.

28. A method according to claim 27 wherein said agent is a COX2 inhibitor or a nonsteroidal anti-inflammatory drug (NSAID).

29. A method according to claim 28 wherein said COX2 inhibitor is selected from the group consisting of Celebrex (Celecoxib), Bextra (Valdecoxib), and Vioxx (Rofecoxib).

30. A method of treating a subject having an increased risk of developing COPD, emphysema, or both COPD and emphysema and for whom a presence of the AA genotype at the 105 C/A polymorphism in the gene encoding Interleukin 18 has been determined, said method comprising administering to said subject an agent capable of augmenting Interleukin 18 activity in said subject.

31. A method of treating a subject having an increased- risk of developing COPD, emphysema, or both COPD and emphysema and for whom a presence of the CC genotype at the −133 G/C polymorphism in the promoter of the gene encoding Interleukin 18 has been determined, said method comprising administering to said subject an agent capable of augmenting Interleukin 18 activity in said subject.

32. A method of treating a subject having an increased risk of developing COPD, emphysema, or both COPD and emphysema and for whom a presence of the 5G5G genotype at the −675 4G/5G polymorphism in the promoter of the gene encoding plasminogen activator inhibitor 1 has been determined, said method comprising administering to said subject an agent capable of augmenting plasminogen activator inhibitor 1 activity in said subject.

33. A method of treating a subject having an increased risk of developing COPD, emphysema, or both COPD and emphysema and for whom a presence of the AA genotype at the 874 A/T polymorphism in the gene encoding interferon-γ has been determined, said method comprising administering to said subject an agent capable of modulating interferon-γ activity in said subject.

34. A method of treating a subject having an increased risk of developing COPD, emphysema, or both COPD and emphysema and for whom a presence of the CC genotype at the −159 C/T polymorphism in the gene encoding CD-14 has been determined, said method comprising administering to said subject an agent capable of modulating CD-14 and/or IgE activity in said subject.

35. An antibody microarray which comprises a substrate presenting antibodies capable of binding to a product of expression of a gene the expression of which is upregulated or downregulated when associated with a susceptibility or protective polymorphism selected from the group defined in claim 1.

36. A method for screening for compounds that modulate the expression and/or activity of a gene, the expression of which is upregulated or downregulated when associated with a susceptibility or protective polymorphism selected from the group defined in claim 2 or claim 3, said method comprising the steps of:
contacting a candidate compound with a cell comprising a susceptibility or protective polymorphism selected from the group defined in claim 2 or claim 3 which has been determined to be associated with the upregulation or downregulation of expression of a gene; and
measuring the expression of said gene following contact with said candidate compound,
wherein a change in the level of expression after the contacting step as compared to before the contacting step is indicative of the ability of the compound to modulate the expression and/or activity of said gene.

37-41. (canceled)

42. A method for screening for compounds that modulate an expression and/or activity of a gene, the expression of which is upregulated or downregulated when associated with a susceptibility or protective polymorphism selected from the group defined in claim 2 or claim 3, said method comprising the steps of:
contacting a candidate compound with a cell comprising a gene, the expression of which is upregulated or downregulated when associated with a susceptibility or protective polymorphism selected from the group defined in claim 2 or claim 3 but which in said cell the expression of which is neither upregulated nor downregulated; and
measuring the expression of said gene following contact with said candidate compound, wherein a change in the level of expression after the contacting step as compared to before the contacting step is indicative of the ability of the compound to modulate the expression and/or activity of said gene.

43-47. (canceled)

48. A method of assessing the likely responsiveness of a subject having an increased risk of or suffering from COPD or emphysema to a prophylactic or therapeutic treatment, which treatment involves restoring a physiologically active concentration of a product of gene expression to be within a range which is normal for an age and sex of the subject, which method comprises detecting in said subject a presence or absence of a susceptibility polymorphism selected from the group defined in claim 3 which when present either upregulates or downregulates expression of said gene such that the physiological active concentration of the expressed gene product is outside said normal range, wherein the detection of the presence of said polymorphism is indicative of the subject likely responding to said treatment.

49. A kit for assessing a subject's risk of developing one or more obstructive lung diseases selected from COPD, emphysema, or both COPD and emphysema, said kit comprising a means of analysing a sample from said subject for a presence or absence of one or more polymorphisms selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881 Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding Matrix Metalloproteinase 1 (MMP1), with reference to the 1G allele only; and
one or more polymorphisms which are in linkage disequilibrium with any one or more of these polymorphisms.


SEQUENCE LISTING (137 sequences)

SEQ ID NO: 1 - DNA, 21 bp, Artificial
  Synthetic nucleic acid sequence
tcgtgagaatgtcttcccatt

SEQ ID NO: 2 - DNA, 29 bp, Artificial Sequence
  Synthetic nucleic acid sequence
tcttggattgatttgagataagtgaaatc

SEQ ID NO: 3 - DNA, 30 bp, Artificial sequence
  Synthetic nucleic acid sequence
acgttggatggcttgttaaccagctttgcc

SEQ ID NO: 4 - DNA, 30 bp, Artificial Sequence
  Synthetic nucleic acid sequence
acgttggatgtttttcagactggcagagcg

SEQ ID NO: 5 - DNA, 30 bp, Artificial Sequence; identical to SEQ ID NO: 4
  Synthetic nucleic acid sequence

SEQ ID NO: 6 - DNA, 30 bp, Artificial Sequence; identical to SEQ ID NO: 3
  Synthetic nucleic acid sequence

SEQ ID NO: 7 - DNA, 30 bp, Artificial Sequence
  Synthetic nucleic acid sequence
acgttggatgcatgtcgccttttcctgctc

SEQ ID NO: 8 - DNA, 30 bp, Artificial Sequence
  Synthetic nucleic acid sequence
acgttggatgcaacacccaacaggcaaatg

SEQ ID NO: 9 - DNA, 30 bp, Artificial Sequence
  Synthetic nucleic acid sequence
acgttggatgtggtggacatggtgaatgac

SEQ ID NO: 10 - DNA, 30 bp, Artificial Sequence
  Synthetic nucleic acid sequence
acgttggatgtggtgcagatgctcacatag

SEQ ID NO: 11 - DNA, 30 bp, Artificial Sequence
  Synthetic nucleic acid sequence
acgttggatgcacagagagagtctggacac

SEQ ID NO: 12 - DNA, 30 bp, Artificial Sequence
  Synthetic nucleic acid sequence
acgttggatgctcttggtctttccctcatc

SEQ ID NO: 13 - DNA, 30 bp, Artificial Sequence
  Synthetic nucleic acid sequence
acgttggatgacagctctgcattcagcacg

SEQ ID NO: 14 - DNA, 30 bp, Artificial Sequence
  Synthetic nucleic acid sequence
acgttggatgagtcaatccctttggtgctc

SEQ ID NO: 15 - DNA, 30 bp, Artificial Sequence
  Synthetic nucleic acid sequence
acgttggatggttttccagcttgcatgtcc

SEQ ID NO: 16 - DNA, 30 bp, Artificial Sequence
  Synthetic nucleic acid sequence
acgttggatgcaatagtcaggtcctgtctc

SEQ ID NO: 17 - DNA, 29 bp, Artificial Sequence
  Synthetic nucleic acid sequence
acgttggatggaacggcagcgccttcttg

SEQ ID NO: 18 - DNA, 30 bp, Artificial Sequence
  Synthetic nucleic acid sequence
acgttggatgacttggcaatggctgtgatg

SEQ ID NO: 19 - DNA, 30 bp, Artificial Sequence
  Synthetic nucleic acid sequence
acgttggatgcagacattcacaattgattt

SEQ ID NO: 20 - DNA, 30 bp, Artificial Sequence
  Synthetic nucleic acid sequence
acgttggatggatagttccaaacatgtgcg

SEQ ID NO: 21 - DNA, 30 bp, Artificial Sequence
  Synthetic nucleic acid sequence
acgttggatggggtattcataagctgaaac

SEQ ID NO: 22 - DNA, 30 bp, Artificial Sequence
  Synthetic nucleic acid sequence
acgttggatgccttcaagttcagtggtcag

SEQ ID NO: 23 - DNA, 30 bp, Artificial Sequence
  Synthetic nucleic acid sequence
acgttggatgggtcaatgaagagaacttgg

SEQ ID NO: 24 - DNA, 30 bp, Artificial Sequence
  Synthetic nucleic acid sequence
acgttggatgaatgtttattgtagaaaacc

SEQ ID NO: 25 - DNA, 15 bp, Artificial Sequence
  Synthetic nucleic acid sequence
agctttgccagttcc

SEQ ID NO: 26 - DNA, 18 bp, Artificial Sequence
  Synthetic nucleic acid sequence
aaaagcaaaattgcctga

SEQ ID NO: 27 - DNA, 15 bp, Artificial Sequence
  Synthetic nucleic acid sequence
tcctgctcttccctc

SEQ ID NO: 28 - DNA, 17 bp, Artificial Sequence
  Synthetic nucleic acid sequence
acctccgctgcaaatac

SEQ ID NO: 29 - DNA, 18 bp, Artificial Sequence
  Synthetic nucleic acid sequence
gagtctggacacgtgggg

SEQ ID NO: 30 - DNA, 19 bp, Artificial Sequence
  Synthetic nucleic acid sequence
tgctgcaggccccagatga

SEQ ID NO: 31 - DNA, 21 bp, Artificial Sequence
  Synthetic nucleic acid sequence
agaaactttttcgcgagggac

SEQ ID NO: 32 - DNA, 24 bp, Artificial Sequence
  Synthetic nucleic acid sequence
agcgccttcttgctggcacccaat

SEQ ID NO: 33 - DNA, 22 bp, Artificial Sequence
  Synthetic nucleic acid sequence
tcttacaacacaaaatcaaatc

SEQ ID NO: 34 - DNA, 15 bp, Artificial Sequence
  Synthetic nucleic acid sequence
agctgaaacttctgg

SEQ ID NO: 35 - DNA, 20 bp, Artificial Sequence
  Synthetic nucleic acid sequence
tcaagcttgccaaagtaatc

SEQ ID NO: 36 - DNA, 16 bp, Artificial Sequence
  Synthetic nucleic acid sequence
agctttgccagttcct

SEQ ID NO: 37 - DNA, 17 bp, Artificial Sequence
  Synthetic nucleic acid sequence
agctttgccagttccgt

SEQ ID NO: 38 - DNA, 19 bp, Artificial Sequence
  Synthetic nucleic acid sequence
aaaagcaaaattgcctgat

SEQ ID NO: 39 - DNA, 21 bp, Artificial Sequence
  Synthetic nucleic acid sequence
aaaagcaaaattgcctgaggc

SEQ ID NO: 40 - DNA, 16 bp, Artificial Sequence
  Synthetic nucleic acid sequence
tcctgctcttccctca

SEQ ID NO: 41 - DNA, 17 bp, Artificial Sequence
  Synthetic nucleic acid sequence
tcctgctcttccctcgt

SEQ ID NO: 42 - DNA, 18 bp, Artificial Sequence
  Synthetic nucleic acid sequence
acctccgctgcaaataca

SEQ ID NO: 43 - DNA, 19 bp, Artificial Sequence
  Synthetic nucleic acid sequence
acctccgctgcaaatacgt

SEQ ID NO: 44 - DNA, 19 bp, Artificial Sequence
  Synthetic nucleic acid sequence
gagtctggacacgtgggga

SEQ ID NO: 45 - DNA, 20 bp, Artificial Sequence
  Synthetic nucleic acid sequence
gagtctggacacgtggggga

SEQ ID NO: 46 - DNA, 20 bp, Artificial Sequence
  Synthetic nucleic acid sequence
tgctgcaggccccagatgat

SEQ ID NO: 47 - DNA, 21 bp, Artificial Sequence
  Synthetic nucleic acid sequence
tgctgcaggccccagatgagc

SEQ ID NO: 48 - DNA, 22 bp, Artificial Sequence
  Synthetic nucleic acid sequence
agaaactttttcgcgagggaca

SEQ ID NO: 49 - DNA, 24 bp, Artificial Sequence
  Synthetic nucleic acid sequence
agaaactttttcgcgagggacggt

SEQ ID NO: 50 - DNA, 25 bp, Artificial Sequence
  Synthetic nucleic acid sequence
agcgccttcttgctggcacccaata

SEQ ID NO: 51 - DNA, 27 bp, Artificial Sequence
  Synthetic nucleic acid sequence
agcgccttcttgctggcacccaatgga

SEQ ID NO: 52 - DNA, 23 bp, Artificial Sequence
  Synthetic nucleic acid sequence
tcttacaacacaaaatcaaatct

SEQ ID NO: 53 - DNA, 24 bp, Artificial Sequence
  Synthetic nucleic acid sequence
tcttacaacacaaaatcaaatcac

SEQ ID NO: 54 - DNA, 16 bp, Artificial Sequence
  Synthetic nucleic acid sequence
agctgaaacttctggc

SEQ ID NO: 55 - DNA, 17 bp, Artificial Sequence
  Synthetic nucleic acid sequence
agctgaaacttctggga

SEQ ID NO: 56 - DNA, 21 bp, Artificial Sequence
  Synthetic nucleic acid sequence
tcaagcttgccaaagtaatct

SEQ ID NO: 57 - DNA, 23 bp, Artificial Sequence
  Synthetic nucleic acid sequence
tcaagcttgccaaagtaatcgga

SEQ ID NO: 58 - DNA, 30 bp, Artificial Sequence
  Synthetic nucleic acid sequence
acgttggatggaagtcagagatgatggcag

SEQ ID NO: 59 - DNA, 30 bp, Artificial Sequence
  Synthetic nucleic acid sequence
acgttggatgatgaatcctggacccaagac

SEQ ID NO: 60 - DNA, 30 bp, Artificial Sequence
  Synthetic nucleic acid sequence
acgttggatggaaagatgtgcgctgatagg

SEQ ID NO: 61 - DNA, 30 bp, Artificial Sequence
  Synthetic nucleic acid sequence
acgttggatggccacatctctttctgcatc

SEQ ID NO: 62 - DNA, 30 bp, Artificial Sequence
  Synthetic nucleic acid sequence
acgttggatgttgcaggtgtcccatcggaa

SEQ ID NO: 63 - DNA, 30 bp, Artificial Sequence
  Synthetic nucleic acid sequence
acgttggatgtagctcgtggtggctgtgca

SEQ ID NO: 64 - DNA, 30 bp, Artificial Sequence
  Synthetic nucleic acid sequence
acgttggatggtgatcacccaaggcttcag

SEQ ID NO: 65 - DNA, 30 bp, Artificial Sequence
  Synthetic nucleic acid sequence
acgttggatggtctgttgactcttttggcc

SEQ ID NO: 66 - DNA, 30 bp, Artificial Sequence
  Synthetic nucleic acid sequence
acgttggatggtagctctccaggcatcaac

SEQ ID NO: 67 - DNA, 30 bp, Artificial Sequence
  Synthetic nucleic acid sequence
acgttggatggtacctggttcccccttttc

SEQ ID NO: 68 - DNA, 30 bp, Artificial Sequence
  Synthetic nucleic acid sequence
acgttggatgtgatcttgttcaccttgccg

SEQ ID NO: 69 - DNA, 30 bp, Artificial Sequence
  Synthetic nucleic acid sequence
acgttggatgagatcgaggtgacgtttgac

SEQ ID NO: 70 - DNA, 30 bp, Artificial Sequence
  Synthetic nucleic acid sequence
acgttggatgagacacagaaccctagatgc

SEQ ID NO: 71 - DNA, 30 bp, Artificial Sequence
  Synthetic nucleic acid sequence
acgttggatggcaatgaaggatgtttcagg

SEQ ID NO: 72 - DNA, 30 bp, Artificial Sequence
  Synthetic nucleic acid sequence
acgttggatgtaagacagctccacagcatc

SEQ ID NO: 73 - DNA, 30 bp, Artificial Sequence
  Synthetic nucleic acid sequence
acgttggatgttccatttcctcaccctcag

SEQ ID NO: 74 - DNA, 30 bp, Artificial Sequence
  Synthetic nucleic acid sequence
acgttggatggatttgtgtgtaggaccctg

SEQ ID NO: 75 - DNA, 30 bp, Artificial Sequence
  Synthetic nucleic acid sequence
acgttggatgggtccccaaaagaaatggag

SEQ ID NO: 76 - DNA, 30 bp, Artificial Sequence
  Synthetic nucleic acid sequence
acgttggatgggattggagaacaaactcac

SEQ ID NO: 77 - DNA, 30 bp, Artificial Sequence
  Synthetic nucleic acid sequence
acgttggatgggcagctgttacaccaaaag

SEQ ID NO: 78 - DNA, 30 bp, Artificial Sequence
  Synthetic nucleic acid sequence
acgttggatgctggcgttttgcaaacatac

SEQ ID NO: 79 - DNA, 30 bp, Artificial Sequence
  Synthetic nucleic acid sequence
acgttggatgttgactggaagaagcaggtg

SEQ ID NO: 80 - DNA, 30 bp, Artificial Sequence
  Synthetic nucleic acid sequence
acgttggatgcctgccaaagaagaaacacc

SEQ ID NO: 81 - DNA, 30 bp, Artificial Sequence
  Synthetic nucleic acid sequence
acgttggatgacgtctgcaggtatgtattc

SEQ ID NO: 82 - DNA, 30 bp, Artificial Sequence
  Synthetic nucleic acid sequence
acgttggatgacttcatccacgtgaagccc

SEQ ID NO: 83 - DNA, 30 bp, Artificial Sequence
  Synthetic nucleic acid sequence
acgttggatgaaactcgtagaaagagccgg

SEQ ID NO: 84 - DNA, 30 bp, Artificial Sequence
  Synthetic nucleic acid sequence
acgttggatgattttctcctcagaggctcc

SEQ ID NO: 85 - DNA, 30 bp, Artificial Sequence
  Synthetic nucleic acid sequence
acgttggatgtgtctgtattgagggtgtgg

SEQ ID NO: 86 - DNA, 30 bp, Artificial Sequence
  Synthetic nucleic acid sequence
acgttggatgttgctggcacccaatggaag

SEQ ID NO: 87 - DNA, 30 bp, Artificial Sequence
  Synthetic nucleic acid sequence
acgttggatgatgagagacatgacgatgcc

SEQ ID NO: 88 - DNA, 30 bp, Artificial Sequence
  Synthetic nucleic acid sequence
acgttggatgactcacagagcacattcacg

SEQ ID NO: 89 - DNA, 30 bp, Artificial Sequence
  Synthetic nucleic acid sequence
acgttggatgtgtcactcgagatcttgagg

SEQ ID NO: 90 - DNA, 17 bp, Artificial Sequence
  Synthetic nucleic acid sequence
gtgcctgtgctgggctc

SEQ ID NO: 91 - DNA, 18 bp, Artificial Sequence
  Synthetic nucleic acid sequence
ggatggagagaaaaaaac

SEQ ID NO: 92 - DNA, 17 bp, Artificial Sequence
  Synthetic nucleic acid sequence
ccctcatgtcatctact

SEQ ID NO: 93 - DNA, 17 bp, Artificial Sequence
  Synthetic nucleic acid sequence
gtcacccactctgttgc

SEQ ID NO: 94 - DNA, 17 bp, Artificial Sequence
  Synthetic nucleic acid sequence
caaagatgggcgtgatg

SEQ ID NO: 95 - DNA, 20 bp, Artificial Sequence
  Synthetic nucleic acid sequence
ccttgccggtgctcttgtcc

SEQ ID NO: 96 - DNA, 20 bp, Artificial Sequence
  Synthetic nucleic acid sequence
cagaatccttcctgttacgg

SEQ ID NO: 97 - DNA, 23 bp, Artificial Sequence
  Synthetic nucleic acid sequence
tccaccaagacttaagttttgct

SEQ ID NO: 98 - DNA, 17 bp, Artificial Sequence
  Synthetic nucleic acid sequence
gaggctgaaccccgtcc

SEQ ID NO: 99 - DNA, 19 bp, Artificial Sequence
  Synthetic nucleic acid sequence
ctttttcatagagtcctgt

SEQ ID NO: 100 - DNA, 19 bp, Artificial Sequence
  Synthetic nucleic acid sequence
ttagtcttgaagtgagggt

SEQ ID NO: 101 - DNA, 22 bp, Artificial Sequence
  Synthetic nucleic acid sequence
tacttatttacgcttgaacctc

SEQ ID NO: 102 - DNA, 17 bp, Artificial Sequence
  Synthetic nucleic acid sequence
ccagctgcccgcaggcc

SEQ ID NO: 103 - DNA, 17 bp, Artificial Sequence
  Synthetic nucleic acid sequence
aattgacagagagctcc

SEQ ID NO: 104 - DNA, 15 bp, Artificial Sequence
  Synthetic nucleic acid sequence
cacgacgtcacgcag

SEQ ID NO: 105 - DNA, 17 bp, Artificial Sequence
  Synthetic nucleic acid sequence
cacattcacggtcacct

SEQ ID NO: 106 - DNA, 18 bp, Artificial Sequence
  Synthetic nucleic acid sequence
gtgcctgtgctgggctca

SEQ ID NO: 107 - DNA, 19 bp, Artificial Sequence
  Synthetic nucleic acid sequence
gtgcctgtgctgggctcgt

SEQ ID NO: 108 - DNA, 19 bp, Artificial Sequence
  Synthetic nucleic acid sequence
ggatggagagaaaaaaaca

SEQ ID NO: 109 - DNA, 20 bp, Artificial Sequence
  Synthetic nucleic acid sequence
ggatggagagaaaaaaacgt

SEQ ID NO: 110 - DNA, 18 bp, Artificial Sequence
  Synthetic nucleic acid sequence
ccctcatgtcatctacta

SEQ ID NO: 111 - DNA, 19 bp, Artificial Sequence
  Synthetic nucleic acid sequence
ccctcatgtcatctactgc

SEQ ID NO: 112 - DNA, 18 bp, Artificial Sequence
  Synthetic nucleic acid sequence
gtcacccactctgttgcc

SEQ ID NO: 113 - DNA, 19 bp, Artificial Sequence
  Synthetic nucleic acid sequence
gtcacccactctgttgcgc

SEQ ID NO: 114 - DNA, 18 bp, Artificial Sequence
  Synthetic nucleic acid sequence
caaagatgggcgtgatga

SEQ ID NO: 115 - DNA, 19 bp, Artificial Sequence
  Synthetic nucleic acid sequence
caaagatgggcgtgatggc

SEQ ID NO: 116 - DNA, 21 bp, Artificial Sequence
  Synthetic nucleic acid sequence
ccttgccggtgctcttgtcca

SEQ ID NO: 117 - DNA, 22 bp, Artificial Sequence
  Synthetic nucleic acid sequence
ccttgccggtgctcttgtccgt

SEQ ID NO: 118 - DNA, 21 bp, Artificial Sequence
  Synthetic nucleic acid sequence
cagaatccttcctgttacggc

SEQ ID NO: 119 - DNA, 22 bp, Artificial Sequence
  Synthetic nucleic acid sequence
cagaatccttcctgttacggtc

SEQ ID NO: 120 - DNA, 24 bp, Artificial Sequence
  Synthetic nucleic acid sequence
tccaccaagacttaagttttgctc

SEQ ID NO: 121 - DNA, 25 bp, Artificial Sequence
  Synthetic nucleic acid sequence
tccaccaagacttaagttttgcttc

SEQ ID NO: 122 - DNA, 18 bp, Artificial Sequence
  Synthetic nucleic acid sequence
gaggctgaaccccgtccc

SEQ ID NO: 123 - DNA, 19 bp, Artificial Sequence
  Synthetic nucleic acid sequence
gaggctgaaccccgtcctc

SEQ ID NO: 124 - DNA, 20 bp, Artificial Sequence
  Synthetic nucleic acid sequence
ctttttcatagagtcctgtt

SEQ ID NO: 125 - DNA, 22 bp, Artificial Sequence
  Synthetic nucleic acid sequence
ctttttcatagagtcctgtaac

SEQ ID NO: 126 - DNA, 20 bp, Artificial Sequence
  Synthetic nucleic acid sequence
ttagtcttgaagtgagggta

SEQ ID NO: 127 - DNA, 21 bp, Artificial Sequence
  Synthetic nucleic acid sequence
ttagtcttgaagtgagggtgt

SEQ ID NO: 128 - DNA, 23 bp, Artificial Sequence
  Synthetic nucleic acid sequence
tacttatttacgcttgaacctca

SEQ ID NO: 129 - DNA, 24 bp, Artificial Sequence
  Synthetic nucleic acid sequence
tacttatttacgcttgaacctcga

SEQ ID NO: 130 - DNA, 18 bp, Artificial Sequence
  Synthetic nucleic acid sequence
ccagctgcccgcaggcca

SEQ ID NO: 131 - DNA, 19 bp, Artificial Sequence
  Synthetic nucleic acid sequence
ccagctgcccgcaggccgt

SEQ ID NO: 132 - DNA, 18 bp, Artificial Sequence
  Synthetic nucleic acid sequence
aattgacagagagctccc

SEQ ID NO: 133 - DNA, 19 bp, Artificial Sequence
  Synthetic nucleic acid sequence
aattgacagagagctcctg

SEQ ID NO: 134 - DNA, 16 bp, Artificial Sequence
  Synthetic nucleic acid sequence
cacgacgtcacgcagc

SEQ ID NO: 135 - DNA, 17 bp, Artificial Sequence
  Synthetic nucleic acid sequence
cacgacgtcacgcagga

SEQ ID NO: 136 - DNA, 18 bp, Artificial Sequence
  Synthetic nucleic acid sequence
cacattcacggtcacctc

SEQ ID NO: 137 - DNA, 19 bp, Artificial Sequence
  Synthetic nucleic acid sequence
cacattcacggtcaccttg